SEQUENCE LISTING (151 sequences)

SEQ ID NO: 1 - RNA, 40 nt, Artificial Sequence
  Sephadex G-100-binding aptamer
guccgaguaauuuacguuuugauacgguugcggaacuugc

SEQ ID NO: 2 - RNA, 42 nt, Human immunodeficiency virus
gguuuaaucagaguagaggagcugacuccuuugguuggacua

SEQ ID NO: 3 - RNA, 98 nt, Artificial Sequence
  Spinach aptamer
gacgcaacugaaugaaauggugaaggacggguccagguguggcugcuucggcagugcagcuuguugaguagagugugagcuccguaacuagucgcguc

SEQ ID NO: 4 - RNA, 103 nt, Artificial Sequence
  Cyan aptamer
gggagacgcaacugaaugaaccuagaguuaugccaggcucugagccugcuucggcaggugcuaugaucgccagcgguaugcaguccguaacuagucgcgucac

SEQ ID NO: 5 - RNA, 34 nt, Human immunodeficiency virus
ggucugggcgcagcgcaagcugacgguacagacc

SEQ ID NO: 6 - RNA, 21 nt, Artificial Sequence
  P22 N aptamer
ggugcgcugacaaagcgcgcc

SEQ ID NO: 7 - RNA, 18 nt, Artificial Sequence
  Lambda N aptamer
gggccugaagaagggccc

SEQ ID NO: 8 - RNA, 28 nt, Bovine immunodeficiency virus
ggcucguguagcucauuagcuccgagcc

SEQ ID NO: 9 - RNA, 27 nt, Human immunodeficiency virus
ccagaucugagccugggagcucucugg

SEQ ID NO: 10 - RNA, 36 nt, Human T-cell lymphotropic virus
gcucaggucgagguacgcaaguaccucccuuggagc

SEQ ID NO: 11 - RNA, 27 nt, Artificial Sequence
  Ricin aptamer
cgaauucaggggacguagcaaugacug

SEQ ID NO: 12-17 - skipped (6 entries)

SEQ ID NO: 18 - protein, 21 aa, Artificial Sequence
  Pep-CD47-self peptide
GNYTCEVTELTREGETIIELK

SEQ ID NO: 19 - protein, 17 aa, Human immunodeficiency virus type 1
TRQARRNRRRRWRERQR

SEQ ID NO: 20 - protein, 17 aa, Bacteriophage P22
NAKTRRHERRRKLAIER

SEQ ID NO: 21 - protein, 22 aa, Bacteriophage lambda
MDAQTRRRERRAEKQAQWKAAN

SEQ ID NO: 22 - protein, 17 aa, Bovine immunodeficiency virus
SGPRPRGTRGKGRRIRR

SEQ ID NO: 23 - protein, 15 aa, Human immunodeficiency virus type 1
SYGRKKRRQRRRPPQ

SEQ ID NO: 24 - protein, 16 aa, Human T-cell lymphotropic virus type 1
MPKTRRRPRRSQRKRP

SEQ ID NO: 25 - protein, 30 aa, Artificial Sequence
  pep-Mir1-CP peptide
SYPVKDTYHPGTGTATARAAAMDVIKMVLA

SEQ ID NO: 26 - protein, 23 aa, Zea mays
TRTPPWPPCPPEEGSGGNGGSHN

SEQ ID NO: 27 - protein, 15 aa, Artificial Sequence
  Citrus Mir1-CP peptide
SVSLEDTYRPGKGTS

SEQ ID NO: 28 - protein, 15 aa, Artificial Sequence
  Apple Mir1-CP peptide
SVSLEDTYRPGKGTS

SEQ ID NO: 29 - protein, 15 aa, Artificial Sequence
  Penetratin peptide
RQIKWFQNRRMKWKK

SEQ ID NO: 30 - protein, 11 aa, Human immunodeficiency virus
YGRKKRRQRRR

SEQ ID NO: 31 - protein, 18 aa, Artificial Sequence
  SynB1 peptide
RGGRLSYSRRRFSTSTGR

SEQ ID NO: 32 - protein, 10 aa, Artificial Sequence
  SynB3 peptide
RRLSYSRRRF

SEQ ID NO: 33 - protein, 12 aa, Artificial Sequence
  PTD-4 peptide
PIRRRKKLRRLK

SEQ ID NO: 34 - protein, 12 aa, Artificial Sequence
  PTD-5 peptide
RRQRRTSKLMKR

SEQ ID NO: 35 - protein, 15 aa, Artificial Sequence
  FHV Coat-(35-49) peptide
RRRRNRTRRNRRRVR

SEQ ID NO: 36 - protein, 19 aa, Artificial Sequence
  BMV Gag-(7-25) peptide
KMTRAQRRAAARRNRWTAR

SEQ ID NO: 37 - protein, 13 aa, Artificial Sequence
  HTLV-II Rex-(4-16) peptide
TRRQRTRRARRNR

SEQ ID NO: 38 - protein, 13 aa, Artificial Sequence
  D-Tat peptide
GRKKRRQRRRPPQ

SEQ ID NO: 39 - protein, 13 aa, Artificial Sequence
  R9-Tat peptide
GRRRRRRRRRPPQ

SEQ ID NO: 40 - protein, 27 aa, Artificial Sequence
  Transportan peptide
GWTLNSAGYLLGKINLKALAALAKKIL

SEQ ID NO: 41 - protein, 17 aa, Artificial Sequence
  MAP peptide
KLALKLALKLALALKLA

SEQ ID NO: 42 - protein, 6 aa, Artificial Sequence
  MAIZE SAP peptide
VRLPPP

SEQ ID NO: 43 - protein, 27 aa, Artificial Sequence
  FBP peptide
GALFLGWLGAAGSTMGAWSQPKKKRKV

SEQ ID NO: 44 - protein, 27 aa, Artificial Sequence
  MPG peptide
  MISC_FEATURE (1)..(1): N-terminal acetyl (Ac) at residue 1
  MISC_FEATURE (27)..(27): C-terminal cysteamide (Cya) at residue 27
GALFLGFLGAAGSTMGAWSQPKKKRKV

SEQ ID NO: 45 - protein, 27 aa, Artificial Sequence
  MPG(deltaNLS) peptide
  MISC_FEATURE (1)..(1): N-terminal acetyl (Ac) at residue 1
  MISC_FEATURE (27)..(27): C-terminal cysteamide (Cya) at residue 27
GALFLGFLGAAGSTMGAWSQPKSKRKV

SEQ ID NO: 46 - protein, 21 aa, Artificial Sequence
  Pep-1 peptide
  MISC_FEATURE (1)..(1): N-terminal acetyl (Ac) at residue 1
  MISC_FEATURE (21)..(21): C-terminal cysteamide (Cya) at residue 27
KETWWETWWTEWSQPKKKRKV

SEQ ID NO: 47 - protein, 21 aa, Artificial Sequence
  Pep-2 peptide
  MISC_FEATURE (1)..(1): N-terminal acetyl (Ac) at residue 1
  MISC_FEATURE (21)..(21): C-terminal cysteamide (Cya) at residue 27
KETWFETWFTEWSQPKKKRKV

SEQ ID NO: 48 - protein, 15 aa, Human immunodeficiency virus
RGWEVLKYWWNLLQY

SEQ ID NO: 49 - protein, 10 aa, Artificial Sequence
  Clathrin-CBM peptide
ETLLDLDFDP

SEQ ID NO: 50 - protein, 15 aa, Artificial Sequence
  Clathrin-4T1 peptide
ETLLDLDFLERPHRD

SEQ ID NO: 51 - protein, 12 aa, Artificial Sequence
  Clathrin-AP2 peptide
GDLLNLDLGPPV

SEQ ID NO: 52 - protein, 14 aa, Artificial Sequence
  Low molecular weight protamine (LMWP) TDSP5 peptide
VSRRRRRRGGRRRR

SEQ ID NO: 53 - protein, 9 aa, Artificial Sequence
  P1Z BPH gut-binding peptide
CHLPRLPQC

SEQ ID NO: 54 - protein, 9 aa, Artificial Sequence
  P2S BPH gut-binding peptide
CLMSSQAAC

SEQ ID NO: 55 - protein, 12 aa, Acyrthosiphon pisum
TCSKKYPRSPCM

SEQ ID NO: 56 - protein, 310 aa, Spodoptera frugiperda
FLDRLSATEDGLHAGRVTFSIAGNDEAAEYFNVLNDGDNSAMLTLKQALPAGVQQFELVIRATDGGTEPGPRSTDCSVTVVFVMTQGDPVFDDNAASVRFVEKEAGMSEKFQLPQADDPKNYRCMDDCHTIYYSIVDGNDGDHFAVEPETNVIYLLKPLDRSQQEQYRVVVAASNTPGGTSTLSSSLLTVTIGVREANPRPIFESEFYTAGVLHTDSIHKELVYLAAKHSEGLPIVYSIDQETMKIDESLQTVVEDAFDINSATGVISLNFQPTSVMHGSFDFEVVASDTRGASDRAKVSIYMISTRVRV

SEQ ID NO: 57 - protein, 310 aa, Manduca sexta
HLERISATDPDGLHAGVVTFQVVGDEESQRYFQVVNDGANLGSLRLLQAVPEEIREFRITIRATDQGTDPGPLSTDMTFRVVFVPTQEPRFASSEHAVAFIEKSAGMEESHQLPLAQDIKNHLCEDDCHSIYYRIIDGNSEGHFGLDPVRNRLFLKKELIREQSASHTLQVAASNSPDFGIPLPASILTVTVTVREADPRPVFMRELYTAGISTADSIGRELLRLHATQSEGAAITYAIDYDTMVVDSLEAVRQSAFVLNAQTGVLTLNIQPTATMHGLFKFEVTATDTAGAQDRTDVTVYVVSSQNRLE

SEQ ID NO: 58 - protein, 377 aa, Diabrotica virgifera
  MISC_FEATURE (353)..(360): Cry3Bb, Cry3Aa binding site
MAVLMTKDLQCSENLNKDGEVGEGIIGEDIDDGDNAKIDFSVLSIVDKETKNDIQESFNISKIDSDYVLNDTLKKVHLIAFEDLKGKYGTYEVTLHMHDEGDPMQTTDPDPTLTLTIEKWNYQTPSIIFPENDQTYIVLSDQQPGQPLALFNNTGTSNTLPDFSATDGETKDYSKWDVKFSYTQTNYEDDKIFVIDHIQPCVSQLQVSKHFNSDLVRSKKYKLTITASVKDGAEQEGEAGYSTSANISIVFLNNDAQPIFQNSDWSVSFVEFNTTQPAKPLEEQAEYENTKGGLPIYYHFYSENQTLSKYFEVDETSGDLSVIGNLTYDYDQDISFHIVASNDSQVRMLDPRSSLNVTVNFLPRNRRAPQWHHHHHH

SEQ ID NO: 59 - protein, 6 aa, Simian virus 40
PKKRKV

SEQ ID NO: 60 - protein, 27 aa, Artificial Sequence
  Chimeric MPG peptide
GLAFLGFLGAAGSTMGAWSQPKKKRKV

SEQ ID NO: 61 - protein, 7 aa, Artificial Sequence
  Apoptotic peptide
KLAKLAK

SEQ ID NO: 62 - protein, 11 aa, Artificial Sequence
  Bp100 peptide
KKLFKKILKYL

SEQ ID NO: 63 - protein, 18 aa, Artificial Sequence
  Tat(2) peptide
RKKRRQRRRRKKRRQRRR

SEQ ID NO: 64 - protein, 35 aa, Artificial Sequence
  Active lycotoxin peptide
HHHHHHDDDKIWLTALKFLGKHAAKHLAKQQLSPW

SEQ ID NO: 65 - protein, 29 aa, Artificial Sequence
  Entry CPP peptide
KHKHKHKHKHKHKHKHKHKKLFKKILKYL

SEQ ID NO: 66 - protein, 30 aa, Homo sapiens
ACYCRIPACIAGERRYGTCIYQGRLWAFCC

SEQ ID NO: 67 - protein, 36 aa, Homo sapiens
DHYNCVSSGGQCLYSACPIFTKIQGTCYRGKAKCCK

SEQ ID NO: 68 - protein, 20 aa, Myrmecia pilosula
GLGSVFGRLARILGRVIPKV

SEQ ID NO: 69 - protein, 41 aa, Struthio camelus
APGNKAECEREKGYCGFLKCSFPFVVSGKCSRFFFCCKNIW

SEQ ID NO: 70 - protein, 48 aa, Artificial Sequence
  PDC1 peptide
ARVCMGKSQHHSFPCISDRLCSNECVKEDGGWTAGYCHLRYCRCQKAC

SEQ ID NO: 71 - protein, 46 aa, Nicotiana alata
RECKTESNTFPGICITKPPCRKACISEKFTDHCSKILRRCLCTKPC

SEQ ID NO: 72 - protein, 54 aa, Heuchera sanguinea
DGVKLCDVPSGTWSGHCGSSSKCSQQCKDREHFAYGGACHYQFPSVKCFCKRQC

SEQ ID NO: 73 - protein, 25 aa, Artificial Sequence
  Lactoferricin B peptide
FKCRRWQWRMKKLGAPSITCVRRAF

SEQ ID NO: 74 - protein, 33 aa, Zea mays
RSGRGECRRQCLRRHEGQPWETQECMRRCRRRG

SEQ ID NO: 75 - protein, 35 aa, Raphanus sativus
KLCQRPSGTWSGVCGNNNACKNQCIRLEKARHGSC

SEQ ID NO: 76 - protein, 18 aa, Artificial Sequence
  Dupont_NH2
RSDSRGFVLSLKKNGAQH

SEQ ID NO: 77 - protein, 26 aa, Artificial Sequence
  DuPont_NH2-Mod
KHKHKHKHRSDSRGFVLSLKKNGAQH

SEQ ID NO: 78 - skipped

SEQ ID NO: 79 - protein, 10 aa, Artificial Sequence
  Florida NPF peptide
ARGPQLRLRF

SEQ ID NO: 80 - DNA, 1404 bp, Ostrinia nubilalis
gggcttcactatgggattgattttattatttgtgttgggtttttgtgccagtgcggtgttcgcgaatgacgataaaatagtagtctgctactatggcacatgggcgacataccggactggcttgggcaagttcgacgtagacgacatcgacccattcctctgcacgcatctggtatatgccttcatcggcattaatgctgaaggaacagctctggcgcttgaccctgagcttgatgttgagagaggtaacttcaagaacttcacttcgttgaaagaaaagaacccgaacctcaaaacgcttgtagctgtcggaggatggagcgaaggatcagctaattactctattatggcagcagaaccagaatatcggcagaacttcatcaacacctctctggcaatgatactggaatacaacttcgatggtctggacgtagactgggagtaccccaatcgcagagacacggtgcatggtgaagacgacattgagaacttcagcactctccttaaggagttgagggaggaattcgataattacggtctactgctgacagtcgctgtggctgctgtggaggaagctgctgtgcagtcctatgacgttccaagtgttgccaagtacgtagactacattggcgtcatgacgtatgacatgcacggcgcctgggactccgtgaccggccacaacgcgcctctgttcataagtgaaggcgagagtgctgaaaacgaaagcactctgtacaacgtcaataacgctgtccagtattggcttagtgcaggatgtcctcccgagaagttagttatgggggttccattctatgggcgcactttcaacctgagtgacccttcagttaacgctccaaattcaccttccaacggagctggtctcgccggtccttacactgctgaaagtggatttatcgggtataatgagttctgctatattctccagaacgagtcttcttggaccgtccagaccgacaaccttgccaaagtgccttacgccttcctcgactacaactgggtatctttcgataacgttgagtccatgaccgccaaagtggagtacgctaacagcttcaaccttcgcggcatcatgctctggagtatcgagactgatgacttccacggcctctgtggagagggaacattccctctgctgaacaccatcaacacagttctagctgaaggctcaacagaagccagacacaacaaccctcctcatcatcaccatcattaaatatggaaacctacttccaggtccttaaaagaagaattaaagacaaatccatagtacaagtattatgaatcttccagagtgccaggagacggcactcttttcataattaacaccaaaaacgaggcaaagataataaaaaatatatttctgcaaaaaaaaaaaaaaaaaaa

SEQ ID NO: 81 - DNA, 894 bp, Ostrinia nubilalis
gcacgagggcaactcgacctaccaagtttgaaaagttcgccgcgcgttccgcacatctatccgcatccaagatgatgagcaagagaatcgccgtcgccatcgccgtggtcctggccgtgatctgcttggctgaggccagggaggagggtcctcacgacatggccgacgccctccgtatgctccaggaactcgaccgctactacactcaagctgctagaccgaggttcggcaagcgatcggatgcgttcacgaactgggctaaggatctcgaaaagccggacttacccgcttggttggcgtacgccagaaggagatgaggggcgcaacagctgccgggtctataagtcactatcaatgttcatgatcactacactatgtaccggactcaatgttaataccgttaatattacacctctttctgtcgttcatgtcacactcgttgtctctgttatcatggtccaaattcaaatttttgaaatataaaaagttactttttaatttagatagatatagatttcatttagaagtttactttttaaatgtttaaatgttttccatttcaataaataaataggttgattgaaaataggctggcatttcaatctcttctattgctgtggtgcttttgtacttaggtgtttatctttattttaatttgtgaaattctagtcaaaacgatagcatttctgtgctatcttctgaaaacaatttgtgccaaagttaacttttacttttacaaaaaaatgattttttggaaaaacctctcgattcaaagtattttaatattattgtttctataatgtaacccagtcgatcaatcccatagtcattagtattcatagaatcttttctgtaactttaagaattattatgtcataatatttgaaccaaaat

SEQ ID NO: 82 - RNA, 67 nt, Artificial Sequence
  Chitinase Divalent MV-RNA 288/874
cguuggaaggugaauuugguuccaaauucaaaagaacccuucaaguggguucuuuucuuucaacguu

SEQ ID NO: 83 - RNA, 67 nt, Artificial Sequence
  Chitinase Divalent MV-RNA 249/1355
uuuugguguuaauuaugaaauauuucauaauugagagagguucaaguccucucucaacaucaagcuu

SEQ ID NO: 84 - RNA, 70 nt, Artificial Sequence
  Chitinase Divalent MV-RNA 750/743
acguuguacagagugcuuuuauaaaagcacuccgucaauaauucaaguuuauugacguuguacagaguuu

SEQ ID NO: 85 - RNA, 70 nt, Artificial Sequence
  Chitinase Divalent MV-RNA 348/341
aauuagcugauccuucgcuguucagcgaaggauuuacucuauuucaaguauagaguaauuagcugauuuu

SEQ ID NO: 86 - RNA, 72 nt, Artificial Sequence
  Chitinase Divalent MV-RNA 79/69
cauucgcgaacaccgcacugguuuaccagugcgggacgauaaauucaaguuuuaucgucauucgcgaauguu

SEQ ID NO: 87 - RNA, 70 nt, Artificial Sequence
  Chitinase Divalent MV-RNA 227/364
cugguucugcugccauaauauuuauauuauggccgcuugaccuucaaguggucaagcgccagagcugguu

SEQ ID NO: 88 - RNA, 416 nt, Artificial Sequence
  Chitinase set linked as ssRNA
  misc_feature (1)..(68): Divalent MV-RNA 288/874
  misc_feature (69)..(134): Divalent MV-RNA 249/1355
  misc_feature (135)..(204): Divalent MV-RNA 750/743
  misc_feature (205)..(274): Divalent MV-RNA 348/341
  misc_feature (275)..(346): Divalent MV-RNA 79/69
  misc_feature (347)..(416): Divalent MV-RNA 227/364
cguuggaaggugaauuugguuccaaauucaaaagaacccuucaaguggguucuuuucuuucaacguuuuuugguguuaauuaugaaauauuucauaauugagagagguucaaguccucucucaacaucaagcuuacguuguacagagugcuuuuauaaaagcacuccgucaauaauucaaguuuauugacguuguacagaguuuaauuagcugauccuucgcuguucagcgaaggauuuacucuauuucaaguauagaguaauuagcugauuuucauucgcgaacaccgcacugguuuaccagugcgggacgauaaauucaaguuuuaucgucauucgcgaauguucugguucugcugccauaauauuuauauuauggccgcuugaccuucaaguggucaagcgccagagcugguu

SEQ ID NO: 89 - RNA, 70 nt, Artificial Sequence
  NPF1 Divalent MV-RNA 818/438
uagugugacaugaacgacagauuucuguuguuccagucgaucuucaagugaucgacuggguuacauuauu

SEQ ID NO: 90 - RNA, 71 nt, Artificial Sequence
  NPF1 Divalent MV-RNA 617/587
auuucaaucaaccuauuuauuuaugaauaaauagcuucuauuguucaagucaauagaagagauugaaauuu

SEQ ID NO: 91 - RNA, 72 nt, Artificial Sequence
  NPF1 Divalent MV-RNA 711/591
acuauuuucaaucaaccuauuuaugaauagguugugaaaacaauuucaaguauuguuuucagaagauaguuu

SEQ ID NO: 92 - RNA, 73 nt, Artificial Sequence
  NPF1 Divalent MV-RNA 388/806
guuacauuauagaaacaauaauauuuguauuauuguguaccggacuucaaguguccgguacauaguguaguuu

SEQ ID NO: 93 - RNA, 73 nt, Artificial Sequence
  NPF1 Divalent MV-RNA 29/215
agcagcuugaguguaguagcggucuugaccgcuacuccuaccaaguucaagucuugguaggucgaguugcuuu

SEQ ID NO: 94 - RNA, 70 nt, Artificial Sequence
  NPF1 Divalent MV-RNA 695/105
ggcgauggcgacggcgauucuugaauugccguuucugugcuuucaaguagcacagaaaugcuaucguuuu

SEQ ID NO: 95 - RNA, 429 nt, Artificial Sequence
  NPF1 set linked as ssRNA
  misc_feature (1)..(70): Divalent MV-RNA 818/438
  misc_feature (71)..(141): Divalent MV-RNA 617/587
  misc_feature (142)..(213): Divalent MV-RNA 711/591
  misc_feature (214)..(286): Divalent MV-RNA 388/806
  misc_feature (287)..(359): Divalent MV-RNA 29/215
  misc_feature (360)..(429): Divalent MV-RNA 695/105
uagugugacaugaacgacagauuucuguuguuccagucgaucuucaagugaucgacuggguuacauuauuauuucaaucaaccuauuuauuuaugaauaaauagcuucuauuguucaagucaauagaagagauugaaauuuacuauuuucaaucaaccuauuuaugaauagguugugaaaacaauuucaaguauuguuuucagaagauaguuuguuacauuauagaaacaauaauauuuguauuauuguguaccggacuucaaguguccgguacauaguguaguuuagcagcuugaguguaguagcggucuugaccgcuacuccuaccaaguucaagucuugguaggucgaguugcuuuggcgauggcgacggcgauucuugaauugccguuucugugcuuucaaguagcacagaaaugcuaucguuuu

SEQ ID NO: 96 - RNA, 845 nt, Artificial Sequence
  Array of linked ECB MV-RNA
cguuggaaggugaauuugguuccaaauucaaaagaacccuucaaguggguucuuuucuuucaacguauuuugguguuaauuaugaaauauuucauaauugagagagguucaaguccucucucaacaucaagcuuacguuguacagagugcuuuuauaaaagcacuccgucaauaauucaaguuuauugacguuguacagaguuuaauuagcugauccuucgcuguucagcgaaggauuuacucuauuucaaguauagaguaauuagcugauuuucauucgcgaacaccgcacugguuuaccagugcgggacgauaaauucaaguuuuaucgucauucgcgaauguucugguucugcugccauaauauuuauauuauggccgcuugaccuucaaguggucaagcgccagagcugguuuagugugacaugaacgacagauuucuguuguuccagucgaucuucaagugaucgacuggguuacauuauuauuucaaucaaccuauuuauuuaugaauaaauagcuucuauuguucaagucaauagaagagauugaaauuuacuauuuucaaucaaccuauuuaugaauagguugugaaaacaauuucaaguauuguuuucagaagauaguuuguuacauuauagaaacaauaauauuuguauuauuguguaccggacuucaaguguccgguacauaguguaguuuagcagcuugaguguaguagcggucuugaccgcuacuccuaccaaguucaagucuugguaggucgaguugcuuuggcgauggcgacggcgauucuugaauugccguuucugugcuuucaaguagcacagaaaugcuaucguuau

SEQ ID NO: 97 - RNA, 1010 nt, Artificial Sequence
  Twelve MV′s w/ 7 nt loops replaced w/ TAT aptamer sequences
guuggaaggugaauuugguuccaaauucaaaagaacccgaucugagccugggagcucucggguucuuuucuuucaacguauuuugguguuaauuaugaaauauuucauaauugagagagggaucugagccugggagcucucccucucucaacaucaagcuuacguuguacagagugcuuuuauaaaagcacuccgucaauaagaucugagccugggagcucucuuauugacguuguacagaguuuaauuagcugauccuucgcuguucagcgaaggauuuacucuaugaucugagccugggagcucucauagaguaauuagcugauuuucauucgcgaacaccgcacugguuuaccagugcgggacgauaaagaucugagccugggagcucucuuuaucgucauucgcgaauguucugguucugcugccauaauauuuauauuauggccgcuugaccgaucugagccugggagcucucggucaagcgccagagcugguuuagugugacaugaacgacagauuucuguuguuccagucgaucgaucugagccugggagcucucgaucgacuggguuacauuauuauuucaaucaaccuauuuauuuaugaauaaauagcuucuauuggaucugagccugggagcucuccaauagaagagauugaaauuuacuauuuucaaucaaccuauuuaugaauagguugugaaaacaaugaucugagccugggagcucucauuguuuucagaagauaguuuguuacauuauagaaacaauaauauuuguauuauuguguaccggacgaucugagccugggagcucucguccgguacauaguguaguuuagcagcuugaguguaguagcggucuugaccgcuacuccuaccaaggaucugagccugggagcucuccuugguaggucgaguugcuuuggcgauggcgacggcgauucuugaauugccguuucugugcugaucugagccugggagcucucagcacagaaaugcuaucguu

SEQ ID NO: 98 - RNA, 997 nt, Artificial Sequence
  ECB-2
  misc_feature (1)..(23): Portion of DNA template for T7 transcription
  misc_feature (917)..(997): Portion of DNA template for T7 transcription
cgcgcguaauacgacucacuauaggggccuagggcaacgccgguccguuggaaggugaauuugguuccaaauucaaaagaacccuucaaguggguucuuuucuuucaacguauuuugguguuaauuaugaaauauuucauaauugagagagguucaaguccucucucaacaucaagcuuacguuguacagagugcuuuuauaaaagcacuccgucaauaauucaaguuuauugacguuguacagaguuuaauuagcugauccuucgcuguucagcgaaggauuuacucuauuucaaguauagaguaauuagcugauuuucauucgcgaacaccgcacugguuuaccagugcgggacgauaaauucaaguuuuaucgucauucgcgaauguucugguucugcugccauaauauuuauauuauggccgcuugaccuucaaguggucaagcgccagagcugguuuagugugacaugaacgacagauuucuguuguuccagucgaucuucaagugaucgacuggguuacauuauuauuucaaucaaccuauuuauuuaugaauaaauagcuucuauuguucaagucaauagaagagauugaaauuuacuauuuucaaucaaccuauuuaugaauagguugugaaaacaauuucaaguauuguuuucagaagauaguuuguuacauuauagaaacaauaauauuuguauuauuguguaccggacuucaaguguccgguacauaguguaguuuagcagcuugaguguaguagcggucuugaccgcuacuccuaccaaguucaagucuugguaggucgaguugcuuuggcgauggcgacggcgauucuugaauugccguuucugugcuuucaaguagcacagaaaugcuaucguuaugaccggcguugccuuaggccgguaccgcaccgaccagaaucaugcaagugcguaagauagucgcgggccggggcgucgacaagcgccgacaaccggcgcuuuuuuug

SEQ ID NO: 99 - RNA, 1165 nt, Artificial Sequence
  ECB-3
  misc_feature (1)..(23): Portion of DNA template for T7 transcription
  misc_feature (1085)..(1165): Portion of DNA template for T7 transcription
cgcgcguaauacgacucacuauaggggccuagggcaacgccgguccguuggaaggugaauuugguuccaaauucaaaagaacccgaucugagccugggagcucucggguucuuuucuuucaacguauuuugguguuaauuaugaaauauuucauaauugagagagggaucugagccugggagcucucccucucucaacaucaagcuuacguuguacagagugcuuuuauaaaagcacuccgucaauaagaucugagccugggagcucucuuauugacguuguacagaguuuaauuagcugauccuucgcuguucagcgaaggauuuacucuaugaucugagccugggagcucucauagaguaauuagcugauuuucauucgcgaacaccgcacugguuuaccagugcgggacgauaaagaucugagccugggagcucucuuuaucgucauucgcgaauguucugguucugcugccauaauauuuauauuauggccgcuugaccgaucugagccugggagcucucggucaagcgccagagcugguuuagugugacaugaacgacagauuucuguuguuccagucgaucgaucugagccugggagcucucgaucgacuggguuacauuauuauuucaaucaaccuauuuauuuaugaauaaauagcuucuauuggaucugagccugggagcucuccaauagaagagauugaaauuuacuauuuucaaucaaccuauuuaugaauagguugugaaaacaaugaucugagccugggagcucucauuguuuucagaagauaguuuguuacauuauagaaacaauaauauuuguauuauuguguaccggacgaucugagccugggagcucucguccgguacauaguguaguuuagcagcuugaguguaguagcggucuugaccgcuacuccuaccaaggaucugagccugggagcucuccuugguaggucgaguugcuuuggcgauggcgacggcgauucuugaauugccguuucugugcugaucugagccugggagcucucagcacagaaaugcuaucguuaugaccggcguugccuuaggccgguaccgcaccgaccagaaucaugcaagugcguaagauagucgcgggccggggcgucgacaagcgccgacaaccggcgcuuuuuuug

SEQ ID NO: 100 - RNA, 61 nt, Artificial Sequence
  Single-TAT aptamer stem 1A
agccgggcagcuccgaccagaucugagccugggagcucucuggucggagcugcccggcuuu

SEQ ID NO: 101 - RNA, 61 nt, Artificial Sequence
  Single-TAT aptamer stem 1b
cagggagcugcggcguccagaucugagccugggagcucucuggacgccgcagcucccuguu

SEQ ID NO: 102 - RNA, 61 nt, Artificial Sequence
  Single-TAT aptamer stem 1c
gaccccgccguagccaccagaucugagccugggagcucucugguggcuacggcggggucuu

SEQ ID NO: 103 - RNA, 61 nt, Artificial Sequence
  Single-TAT aptamer stem 2a
cgacgggcagcucggaccagaucugagccugggagcucucugguccgagcugcccgucguu

SEQ ID NO: 104 - RNA, 61 nt, Artificial Sequence
  Single-TAT aptamer stem 2b
gacggaucugcggcguccagaucugagccugggagcucucuggacgccgcagauccgucuu

SEQ ID NO: 105 - RNA, 61 nt, Artificial Sequence
  Single-TAT aptamer stem 2c
gccauugccguagccaccagaucugagccugggagcucucugguggcuacggcaauggcuu

SEQ ID NO: 106 - RNA, 65 nt, Artificial Sequence
  BIV aptamer stem 1a
  misc_feature (17)..(47): BIV TAR
agccgggcagcuccgagaggcggugaacuuggaaucccacaagggcgucggagcugcccggcuuu

SEQ ID NO: 107 - RNA, 65 nt, Artificial Sequence
  BIV aptamer stem 1b
  misc_feature (17)..(47): BIV TAR
cagggagcugcggcgugaggcggugaacuuggaaucccacaagggcgacgccgcagcucccuguu

SEQ ID NO: 108 - RNA, 65 nt, Artificial Sequence
  BIV aptamer stem 1c
  misc_feature (17)..(47): BIV TAR
gaccccgccguagccagaggcggugaacuuggaaucccacaagggcguggcuacggcggggucuu

SEQ ID NO: 109 - RNA, 65 nt, Artificial Sequence
  BIV aptamer stem 2a
  misc_feature (17)..(47): BIV TAR
cgacgggcagcucggagaggcggugaacuuggaaucccacaagggcguccgagcugcccgucguu

SEQ ID NO: 110 - RNA, 65 nt, Artificial Sequence
  BIV aptamer stem 2b
  misc_feature (17)..(47): BIV TAR
gacggaucugcggcgugaggcggugaacuuggaaucccacaagggcgacgccgcagauccgucuu

SEQ ID NO: 111 - RNA, 65 nt, Artificial Sequence
  BIV aptamer stem 2c
  misc_feature (17)..(47): BIV TAR
gccauugccguagccagaggcggugaacuuggaaucccacaagggcguggcuacggcaauggcuu

SEQ ID NO: 112 - RNA, 1914 nt, Artificial Sequence
  FAW-4
  misc_feature (1)..(23): Portion of DNA template for T7 transcription
  misc_feature (124)..(202): Divalent vATPa_545/303
  misc_feature (203)..(279): Divalent vATPa_320/501
  misc_feature (280)..(340): TAT aptamer stem 1a
  misc_feature (341)..(417): Divalent vATPa_542/388
  misc_feature (418)..(478): TAT aptamer stem 1b
  misc_feature (479)..(554): Divalent COPI_SB_1241/1108
  misc_feature (555)..(633): Divalent COPI_BP_1525/1323
  misc_feature (634)..(710): Divalent COPI_BP_1448/1490
  misc_feature (711)..(771): TAT aptamer stem 1c
  misc_feature (771)..(848): Divalent COPI_BP_1532/1446
  misc_feature (927)..(1005): DIvalent vATPa_545/303
  misc_feature (1006)..(1082): DIvalent vATPa_320/501
  misc_feature (1083)..(1143): TAT aptamer stem 2a
  misc_feature (1144)..(1220): Divalent vATPa_542/388
  misc_feature (1300)..(1376): Divalent COPI_SB_1419/1240
  misc_feature (1377)..(1437): TAT aptamer stem 2b
  misc_feature (1438)..(1513): Divalent COPI_SB_1241/1108
  misc_feature (1514)..(1592): Divalent COPI_BP_1525/1323
  misc_feature (1593)..(1669): Divalent COPI_BP_1448/1490
  misc_feature (1670)..(1730): TAT aptamer stem 2c
  misc_feature (1730)..(1807): Divalent COPI_BP_1532/1446
  misc_feature (1834)..(1914): Portion of DNA template for T7 transcription
cgcgcguaauacgacucacuauaggggccuagggcaacgccggucccuucaguggccgcuggauguuucaaguacguccggcacgcucguagaguucaagucugugugagcucguugaugguugguaucguacucccuuguguuucaaguacacaagggaagagagagucgauucaaguuguguucucguguacgauaccuuaagcugcccgagcauaagauuucaaguaucuuaugcuuaaccaaucuuucaaguggggugguuggcgggcagcuuuuagccgggcagcuccgaccagaucugagccugggagcucucuggucggagcugcccggcuuucggcaacuacucuucccuuguucaagucaagggaagagagagucgagguucaagucagugacuuuguaguugccguucagggagcugcggcguccagaucugagccugggagcucucuggacgccgcagcucccuguuugcgacacuuucuaugauuuucaaguaaucauagaaggagaacgccauucaaguuggaguucucaagugucgcauuucauaucuaccgacagcuuguucaagucaagcugucguggagcugauguucaagucaacaguuucugguagauaugauuggaagaauccucauaccuucuucaagugaagguaugacguuggcgacuucaagugcugccgacucggauucuuccuugaccccgccguagccaccagaucugagccugggagcucucugguggcuacggcggggucuuguauucgccuugucaccauucaaguuggugacaagcugucguggcuuucaaguagguaugacguuggcgaguacauuccuucaguggccgcuggauguuucaaguacguccggcacgcucguagaguucaagucugugugagcucguugaugguugguaucguacucccuuguguuucaaguacacaagggaagagagagucgauucaaguuguguucucguguacgauaccuuaagcugcccgagcauaagauuucaaguaucuuaugcuuaaccaaucuuucaaguggggugguuggcgggcagcuuuucgacgggcagcucggaccagaucugagccugggagcucucugguccgagcugcccgucguucggcaacuacucuucccuuguucaagucaagggaagagagagucgagguucaagucagugacuuuguaguugccguuagucccauauuuugacgagguuucaaguacguuguuagagccgugcaucuucaagugauguauggcugugugggauuaauggcguucuccggcaucgaacucaaguuucgaugccgucuucugugauuucaaguaucauagaaggagaacgccauugacggaucugcggcguccagaucugagccugggagcucucuggacgccgcagauccgucuuugcgacacuuucuaugauuuucaaguaaucauagaaggagaacgccauucaaguuggaguucucaagugucgcauuucauaucuaccgacagcuuguucaagucaagcugucguggagcugauguucaagucaacaguuucugguagauaugauuggaagaauccucauaccuucuucaagugaagguaugacguuggcgacuucaagugcugccgacucggauucuuccuugccauugccguagccaccagaucugagccugggagcucucugguggcuacggcaauggcuuguauucgccuugucaccauucaaguuggugacaagcugucguggcuuucaaguagguaugacguuggcgaguacauugaccggcguugccuuaggccgguaccgcaccgaccagaaucaugcaagugcguaagauagucgcgggccggggcgucgacaagcgccgacaaccggcgcuuuuuuug

SEQ ID NO: 113 - RNA, 1082 nt, Artificial Sequence
  Exemplary aptamer layout to create multi-domain nanoparticle shells
  misc_feature (375)..(405): BIV TAR
  misc_feature (427)..(457): BIV TAR
  misc_feature (501)..(531): BIV TAR
  misc_feature (553)..(583): BIV TAR
  misc_feature (625)..(655): BIV TAR
  misc_feature (676)..(706): BIV TAR
  misc_feature (749)..(779): BIV TAR
  misc_feature (801)..(831): BIV TAR
  misc_feature (903)..(933): BIV TAR
  misc_feature (976)..(1006): BIV TAR
  misc_feature (1028)..(1058): BIV TAR
gguaucguacucccuuguguccagaucugagccugggagcucucuggacacaagggaagagagagucgaccagaucugagccugggagcucucugguguguucucguguacgauaccuuaagcugcccgagcauaagauccagaucugagccugggagcucucuggaucuuaugcuuaaccaaucuccagaucugagccugggagcucucuggggggugguuggcgggcagcuuuucggcaacuacucuucccuugccagaucugagccugggagcucucuggcaagggaagagagagucgaggccagaucugagccugggagcucucuggcagugacuuuguaguugccguuagucccauauuuugacgaggugaggcggugaacuuggaaucccacaagggcgacguuguuagagccgugcaucgaggcggugaacuuggaaucccacaagggcggauguauggcugugugggauuaauggcguucuccggcaucgaagaggcggugaacuuggaaucccacaagggcguucgaugccgucuucugugaugaggcggugaacuuggaaucccacaagggcgaucauagaaggagaacgccauuugcgacacuuucuaugauugaggcggugaacuuggaaucccacaagggcgaucauagaaggagaacgccagaggcggugaacuuggaaucccacaagggcguggaguucucaagugucgcauuucauaucuaccgacagcuuggaggcggugaacuuggaaucccacaagggcgcaagcugucguggagcugauggaggcggugaacuuggaaucccacaagggcgcaacaguuucugguagauaugauuggaagaauccucauaccuucuucaagugaagguaugacguuggcgacgaggcggugaacuuggaaucccacaagggcggcugccgacucggauucuuccuuuguauucgccuugucaccagaggcggugaacuuggaaucccacaagggcguggugacaagcugucguggcugaggcggugaacuuggaaucccacaagggcgagguaugacguuggcgaguacauu

SEQ ID NO: 114 - RNA, 1234 nt, Artificial Sequence
  FAW-5
  misc_feature (1)..(23): Portion of DNA template for T7 transcription
  misc_feature (420)..(450): BIV TAR
  misc_feature (472)..(502): BIV TAR
  misc_feature (546)..(576): BIV TAR
  misc_feature (598)..(628): BIV TAR
  misc_feature (670)..(700): BIV TAR
  misc_feature (721)..(751): BIV TAR
  misc_feature (794)..(824): BIV TAR
  misc_feature (846)..(876): BIV TAR
  misc_feature (948)..(978): BIV TAR
  misc_feature (1021)..(1051): BIV TAR
  misc_feature (1073)..(1103): BIV TAR
  misc_feature (1153)..(1234): Portion of DNA template for T7 transcription
cgcgcguaauacgacucacuauaggggccuagggcaacgccggucgguaucguacucccuuguguccagaucugagccugggagcucucuggacacaagggaagagagagucgaccagaucugagccugggagcucucugguguguucucguguacgauaccuuaagcugcccgagcauaagauccagaucugagccugggagcucucuggaucuuaugcuuaaccaaucuccagaucugagccugggagcucucuggggggugguuggcgggcagcuuuucggcaacuacucuucccuugccagaucugagccugggagcucucuggcaagggaagagagagucgaggccagaucugagccugggagcucucuggcagugacuuuguaguugccguuagucccauauuuugacgaggugaggcggugaacuuggaaucccacaagggcgacguuguuagagccgugcaucgaggcggugaacuuggaaucccacaagggcggauguauggcugugugggauuaauggcguucuccggcaucgaagaggcggugaacuuggaaucccacaagggcguucgaugccgucuucugugaugaggcggugaacuuggaaucccacaagggcgaucauagaaggagaacgccauuugcgacacuuucuaugauugaggcggugaacuuggaaucccacaagggcgaucauagaaggagaacgccagaggcggugaacuuggaaucccacaagggcguggaguucucaagugucgcauuucauaucuaccgacagcuuggaggcggugaacuuggaaucccacaagggcgcaagcugucguggagcugauggaggcggugaacuuggaaucccacaagggcgcaacaguuucugguagauaugauuggaagaauccucauaccuucuucaagugaagguaugacguuggcgacgaggcggugaacuuggaaucccacaagggcggcugccgacucggauucuuccuuuguauucgccuugucaccagaggcggugaacuuggaaucccacaagggcguggugacaagcugucguggcugaggcggugaacuuggaaucccacaagggcgagguaugacguuggcgaguacauugaccggcguugccuuaggccgguaccgcaccgaccagaaucaugcaagugcguaagauagucgcgggccggggcgucgacaagcgccgacaaccggcgcuuuuuuug

SEQ ID NO: 115 - RNA, 353 nt, Artificial Sequence
  1X TAT-aptamer region
gguaucguacucccuuguguccagaucugagccugggagcucucuggacacaagggaagagagagucgaccagaucugagccugggagcucucugguguguucucguguacgauaccuuaagcugcccgagcauaagauccagaucugagccugggagcucucuggaucuuaugcuuaaccaaucuccagaucugagccugggagcucucuggggggugguuggcgggcagcuuuucggcaacuacucuucccuugccagaucugagccugggagcucucuggcaagggaagagagagucgaggccagaucugagccugggagcucucuggcagugacuuuguaguugccguu

SEQ ID NO: 116 - RNA, 729 nt, Artificial Sequence
  2X BIV-aptamer region
agucccauauuuugacgaggugaggcggugaacuuggaaucccacaagggcgacguuguuagagccgugcaucgaggcggugaacuuggaaucccacaagggcggauguauggcugugugggauuaauggcguucuccggcaucgaagaggcggugaacuuggaaucccacaagggcguucgaugccgucuucugugaugaggcggugaacuuggaaucccacaagggcgaucauagaaggagaacgccauuugcgacacuuucuaugauugaggcggugaacuuggaaucccacaagggcgaucauagaaggagaacgccagaggcggugaacuuggaaucccacaagggcguggaguucucaagugucgcauuucauaucuaccgacagcuuggaggcggugaacuuggaaucccacaagggcgcaagcugucguggagcugauggaggcggugaacuuggaaucccacaagggcgcaacaguuucugguagauaugauuggaagaauccucauaccuucuucaagugaagguaugacguuggcgacgaggcggugaacuuggaaucccacaagggcggcugccgacucggauucuuccuuuguauucgccuugucaccagaggcggugaacuuggaaucccacaagggcguggugacaagcugucguggcugaggcggugaacuuggaaucccacaagggcgagguaugacguuggcgaguacauu

SEQ ID NO: 117 - skipped

SEQ ID NO: 118 - RNA, 1963 nt, Artificial Sequence
  FAW-6
  misc_feature (1)..(23): Portion of DNA template for T7 transcription
  misc_feature (46)..(398): TAT-aptamer region
  misc_feature (399)..(1127): BIV-aptamer region
  misc_feature (1128)..(1856): BIV-aptamer region
  misc_feature (1882)..(1963): Portion of DNA template for T7 transcription
cgcgcguaauacgacucacuauaggggccuagggcaacgccggucgguaucguacucccuuguguccagaucugagccugggagcucucuggacacaagggaagagagagucgaccagaucugagccugggagcucucugguguguucucguguacgauaccuuaagcugcccgagcauaagauccagaucugagccugggagcucucuggaucuuaugcuuaaccaaucuccagaucugagccugggagcucucuggggggugguuggcgggcagcuuuucggcaacuacucuucccuugccagaucugagccugggagcucucuggcaagggaagagagagucgaggccagaucugagccugggagcucucuggcagugacuuuguaguugccguuagucccauauuuugacgaggugaggcggugaacuuggaaucccacaagggcgacguuguuagagccgugcaucgaggcggugaacuuggaaucccacaagggcggauguauggcugugugggauuaauggcguucuccggcaucgaagaggcggugaacuuggaaucccacaagggcguucgaugccgucuucugugaugaggcggugaacuuggaaucccacaagggcgaucauagaaggagaacgccauuugcgacacuuucuaugauugaggcggugaacuuggaaucccacaagggcgaucauagaaggagaacgccagaggcggugaacuuggaaucccacaagggcguggaguucucaagugucgcauuucauaucuaccgacagcuuggaggcggugaacuuggaaucccacaagggcgcaagcugucguggagcugauggaggcggugaacuuggaaucccacaagggcgcaacaguuucugguagauaugauuggaagaauccucauaccuucuucaagugaagguaugacguuggcgacgaggcggugaacuuggaaucccacaagggcggcugccgacucggauucuuccuuuguauucgccuugucaccagaggcggugaacuuggaaucccacaagggcguggugacaagcugucguggcugaggcggugaacuuggaaucccacaagggcgagguaugacguuggcgaguacauuagucccauauuuugacgaggugaggcggugaacuuggaaucccacaagggcgacguuguuagagccgugcaucgaggcggugaacuuggaaucccacaagggcggauguauggcugugugggauuaauggcguucuccggcaucgaagaggcggugaacuuggaaucccacaagggcguucgaugccgucuucugugaugaggcggugaacuuggaaucccacaagggcgaucauagaaggagaacgccauuugcgacacuuucuaugauugaggcggugaacuuggaaucccacaagggcgaucauagaaggagaacgccagaggcggugaacuuggaaucccacaagggcguggaguucucaagugucgcauuucauaucuaccgacagcuuggaggcggugaacuuggaaucccacaagggcgcaagcugucguggagcugauggaggcggugaacuuggaaucccacaagggcgcaacaguuucugguagauaugauuggaagaauccucauaccuucuucaagugaagguaugacguuggcgacgaggcggugaacuuggaaucccacaagggcggcugccgacucggauucuuccuuuguauucgccuugucaccagaggcggugaacuuggaaucccacaagggcguggugacaagcugucguggcugaggcggugaacuuggaaucccacaagggcgagguaugacguuggcgaguacauugaccggcguugccuuaggccgguaccgcaccgaccagaaucaugcaagugcguaagauagucgcgggccggggcgucgacaagcgccgacaaccggcgcuuuuuuug

SEQ ID NO: 119 - protein, 188 aa, Artificial Sequence
  TAT_MAIZE_RIP2_N
MHKHKYGRKKRRQRRRAAAAMAEPNPELSGLITQTKKKNIVPKFTEIFPVEDTAYPYSAFITSVRKEVIKYCTNHTGIVQPVLPLEKNVPELWFYTELKTKTRSITLAIRMDNLYLVGFRTPGGVWWEFGKDGDTHLLDDNAKWLGFGGRYQDLIGSKGLETVTMGRAEMTTAVNYLAKKTTHHHHHH

SEQ ID NO: 120 - protein, 123 aa, Artificial Sequence
  TAT_MAIZE_RIP2_C
MHKHKYGRKKRRQRRRAAAAADPKAEEKSNLAKLVIMVCEGLRFFTVSRKVDEGFKKPQAVTISALEGKQVQKWDRISKAVFRWAVDPTAEIPDMKDLGIKDKNAAAQIVALVKDQNHHHHHH

SEQ ID NO: 121 - protein, 194 aa, Artificial Sequence
  BIV_RIP2_N
MHKHKSGPRPRGTRGKGRRIRRAAAAMAEPNPELSGLITQTKKKNIVPKFTEIFPVEDTAYPYSAFITSVRKEVIKYCTNHTGIVQPVLPLEKNVPELWFYTELKTKTRSITLAIRMDNLYLVGFRTPGGVWWEFGKDGDTHLLDDNAKWLGFGGRYQDLIGSKGLETVTMGRAEMTTAVNYLAKKTTHHHHHH

SEQ ID NO: 122 - protein, 129 aa, Artificial Sequence
  BIV_RIP2_C
MHKHKSGPRPRGTRGKGRRIRRAAAAADPKAEEKSNLAKLVIMVCEGLRFFTVSRKVDEGFKKPQAVTISALEGKQVQKWDRISKAVFRWAVDPTAEIPDMKDLGIKDKNAAAQIVALVKDQNHHHHHH

SEQ ID NO: 123 - protein, 131 aa, Artificial Sequence
  TAT_SNOW_LECTIN
MHKHKYGRKKRRQRRRAAAADNILYSGETLSTGEFLNYGSFVFIMQEDCNLVLYDVDKPIWATNTGGLSRSCFLSMQTDGNLVVYNPSNKPIWASNTGGQNGNYVCILQKDRNVVIYGTDRWATGHHHHHH

SEQ ID NO: 124 - protein, 137 aa, Artificial Sequence
  BIV_SNOW_LECTIN
MHKHKSGPRPRGTRGKGRRIRRAAAADNILYSGETLSTGEFLNYGSFVFIMQEDCNLVLYDVDKPIWATNTGGLSRSCFLSMQTDGNLVVYNPSNKPIWASNTGGQNGNYVCILQKDRNVVIYGTDRWATGHHHHHH

SEQ ID NO: 125 - protein, 415 aa, Artificial Sequence
  TAT-Maize mir1
YGRKKRRQRRRMRPTRSAVSATALLLLAVALALAATAAARHSYTTTTTRVPAPAERADEEVRRMYEAWKSKHGRGGSSNDDCDMAPGDDEQEEEDRRLRLEVFRDNLRYIDAHNAEADAGLHTFRLGLTPFADLTLEEYRGRVLGFRARGRRSGARYGSGYSVRGGDLPDAIDWRQLGAVTEVKDQQQCGGCWAFSAVAAIEGVNAIATGNLVSLSEQEIIDCDAQDSGCDGGQMENAFRFVIGNGGIDTEADYPFIGTDGTCDASKEKNEKVATIDGLVEVASNNETALQEAVAIQPVSVAIDASGRAFQHYSSGIFNGPCGTSLDHGVTAVGYGSESGKDYWIVKNSWSASWGEAGYIRMRRNVPRPTGKCGIAMDASYPVKDTYHPGTGTATARAAAMDVIKMVLAHHHHHH

SEQ ID NO: 126 - protein, 29 aa, Artificial Sequence
  TAT_CRY2
YGRKKRRQRRRCHLPRLPQCCHLPRLPQC

SEQ ID NO: 127 - protein, 29 aa, Artificial Sequence
  CM-TAT
KWKLFKKIGAVLKVLTTGYGRKKRRQRRR

SEQ ID NO: 128 - protein, 33 aa, Artificial Sequence
  E5-TAT
GLFEAIAEFIENGWEGLIEGWYGGRKKRRQRRR

SEQ ID NO: 129 - protein, 469 aa, Artificial Sequence
  BIV_DENSO-VP4
MHKHKSGPRPRGTRGKGRRIRRAAAAMSLPGTGSGTSSGGGNTSGQEVYVIPRPFSNFGKKLSTYTKSHKFMIFGLANNVIGPTGTGTTAVNRLITTCLAEIPWQKLPLYMNQSEFDLLPPGSRVVECNVKVIFRTNRIAFETSSTATKQATLNQISNLQTAVGLNKLGWGIDRSFTAFQSDQPMIPTATSAPKYEPITGTTGYRGMIADYYGADSTNDAAFGNAGNYPHHQVGSFTFIQNYYCMYQQTNQGTGGWPCLAEHLQQFDSKTVNNQCLIDVTYKPKMGLIKPPLNYKIIGQPTAKGTISVGDNLVNMRGAVVINPPEATQSVTESTHNLTRNFPANLFNIYSDIEKSQILHKGPWGHENPQIQPSVHIGIQAVPALTTGALLVNSSPLNSWTDSMGYIDVMSSCTVMESQPTHFPFSTDANTNPGNTIYRINLTPNSLTSAFNGLYGNGATLGNVHHHHHH

SEQ ID NO: 130 - protein, 463 aa, Artificial Sequence
  TAT_DENSO-VP4
MHKHKYGRKKRRQRRRAAAAMSLPGTGSGTSSGGGNTSGQEVYVIPRPFSNFGKKLSTYTKSHKFMIFGLANNVIGPTGTGTTAVNRLITTCLAEIPWQKLPLYMNQSEFDLLPPGSRVVECNVKVIFRTNRIAFETSSTATKQATLNQISNLQTAVGLNKLGWGIDRSFTAFQSDQPMIPTATSAPKYEPITGTTGYRGMIADYYGADSTNDAAFGNAGNYPHHQVGSFTFIQNYYCMYQQTNQGTGGWPCLAEHLQQFDSKTVNNQCLIDVTYKPKMGLIKPPLNYKIIGQPTAKGTISVGDNLVNMRGAVVINPPEATQSVTESTHNLTRNFPANLFNIYSDIEKSQILHKGPWGHENPQIQPSVHIGIQAVPALTTGALLVNSSPLNSWTDSMGYIDVMSSCTVMESQPTHFPFSTDANTNPGNTIYRINLTPNSLTSAFNGLYGNGATLGNVHHHHHH

SEQ ID NO: 131 - RNA, 85 nt, Artificial Sequence
  Dual-TAT aptamer stem 1A
gggcagcuccgaaaccagaucugagccugggagcucucuggacgccaucugagccugggagcucuggcgaaucggagcugcccuu

SEQ ID NO: 132 - RNA, 85 nt, Artificial Sequence
  Dual-TAT aptamer stem 1B
gagcugcggcguaaccagaucugagccugggagcucucuggacgccaucugagccugggagcucuggcgaaacgccgcagcucuu

SEQ ID NO: 133 - RNA, 85 nt, Artificial Sequence
  Dual-TAT aptamer stem 1C
ccgccguagccaaaccagaucugagccugggagcucucuggacgccaucugagccugggagcucuggcgaauggcuacggcgguu

SEQ ID NO: 134 - RNA, 85 nt, Artificial Sequence
  Dual-TAT aptamer stem 2A
gggcagcucggaaaccagaucugagccugggagcucucuggacgccaucugagccugggagcucuggcgaauccgagcugcccuu

SEQ ID NO: 135 - RNA, 85 nt, Artificial Sequence
  Dual-TAT aptamer stem 2B
gaucugcggcguaaccagaucugagccugggagcucucuggacgccaucugagccugggagcucuggcgaaacgccgcagaucuu

SEQ ID NO: 136 - RNA, 85 nt, Artificial Sequence
  Dual-TAT aptamer stem 2C
uugccguagccaaaccagaucugagccugggagcucucuggacgccaucugagccugggagcucuggcgaauggcuacggcaauu

SEQ ID NO: 137 - RNA, 2438 nt, Artificial Sequence
  ECB-1
  misc_feature (1)..(23): Portion of DNA template for T7 transcription
  misc_feature (250)..(334): Dual-TAT Aptamer Stem 1a
  misc_feature (547)..(631): Dual-TAT Aptamer Stem 1b
  misc_feature (845)..(929): Dual-TAT Aptamer Stem 1c
  misc_feature (1146)..(1230): Dual-TAT Aptamer Stem 2a
  misc_feature (1435)..(1519): Dual-TAT Aptamer Stem 2b
  misc_feature (1732)..(1816): Dual-TAT Aptamer Stem 2c
  misc_feature (2130)..(2114): Dual-TAT Aptamer Stem 1c
  misc_feature (2357)..(2438): Portion of DNA template for T7 transcription
cgcgcguaauacgacucacuauaggggccuagggcaacgccgguccguuggaaggugaauuugguuccaaauucaaaagaacccuucaaguggguucuuuucuuucaacguauuuugguguuaauuaugaaauauuucauaauugagagagguucaaguccucucucaacaucaagcuuacguuguacagagugcuuuuauaaaagcacuccgucaauaauucaaguuuauugacguuguacagaguuugggcagcuccgaaaccagaucugagccugggagcucucuggacgccaucugagccugggagcucuggcgaaucggagcugcccuuaauuagcugauccuucgcuguucagcgaaggauuuacucuauuucaaguauagaguaauuagcugauuuucauucgcgaacaccgcacugguuuaccagugcgggacgauaaauucaaguuuuaucgucauucgcgaauguucugguucugcugccauaauauuuauauuauggccgcuugaccuucaaguggucaagcgccagagcugguugagcugcggcguaaccagaucugagccugggagcucucuggacgccaucugagccugggagcucuggcgaaacgccgcagcucuuuagugugacaugaacgacagauuucuguuguuccagucgaucuucaagugaucgacuggguuacauuauuauuucaaucaaccuauuuauuuaugaauaaauagcuucuauuguucaagucaauagaagagauugaaauuuacuauuuucaaucaaccuauuuaugaauagguugugaaaacaauuucaaguauuguuuucagaagauaguuuccgccguagccaaaccagaucugagccugggagcucucuggacgccaucugagccugggagcucuggcgaauggcuacggcgguuguuacauuauagaaacaauaauauuuguauuauuguguaccggacuucaaguguccgguacauaguguaguuuagcagcuugaguguaguagcggucuugaccgcuacuccuaccaaguucaagucuugguaggucgaguugcuuuggcgauggcgacggcgauucuugaauugccguuucugugcuuucaaguagcacagaaaugcuaucguuuugggcagcucggaaaccagaucugagccugggagcucucuggacgccaucugagccugggagcucuggcgaauccgagcugcccuucguuggaaggugaauuugguuccaaauucaaaagaacccuucaaguggguucuuuucuuucaacguauuuugguguuaauuaugaaauauuucauaauugagagagguucaaguccucucucaacaucaagcuuacguuguacagagugcuuuuauaaaagcacuccgucaauaauucaaguuuauugacguuguacagaguuugaucugcggcguaaccagaucugagccugggagcucucuggacgccaucugagccugggagcucuggcgaaacgccgcagaucuuaauuagcugauccuucgcuguucagcgaaggauuuacucuauuucaaguauagaguaauuagcugauuuucauucgcgaacaccgcacugguuuaccagugcgggacgauaaauucaaguuuuaucgucauucgcgaauguucugguucugcugccauaauauuuauauuauggccgcuugaccuucaaguggucaagcgccagagcugguuuugccguagccaaaccagaucugagccugggagcucucuggacgccaucugagccugggagcucuggcgaauggcuacggcaauuuagugugacaugaacgacagauuucuguuguuccagucgaucuucaagugaucgacuggguuacauuauuauuucaaucaaccuauuuauuuaugaauaaauagcuucuauuguucaagucaauagaagagauugaaauuuacuauuuucaaucaaccuauuuaugaauagguugugaaaacaauuucaaguauuguuuucagaagauaguuuccgccguagccaaaccagaucugagccugggagcucucuggacgccaucugagccugggagcucuggcgaauggcuacggcgguuguuacauuauagaaacaauaauauuuguauuauuguguaccggacuucaaguguccgguacauaguguaguuuagcagcuugaguguaguagcggucuugaccgcuacuccuaccaaguucaagucuugguaggucgaguugcuuuggcgauggcgacggcgauucuugaauugccguuucugugcuuucaaguagcacagaaaugcuaucguuuugaccggcguugccuuaggccgguaccgcaccgaccagaaucaugcaagugcguaagauagucgcgggccggggcgucgacaagcgccgacaaccggcgcuuuuuuuug

SEQ ID NO: 138 - DNA, 1125 bp, Spodoptera frugiperda
ggtgacatggccaccatccaggtatacgaagaaacatcaggtgtaactgtaggtgaccccgtgctgcgtaccggcaagcccctgtccgtagagctgggtcctggtatcctcggctccatctttgacggtatccagcggccactgaaggacatcaacgagctcacacagtccatctacatccccaagggtgtcaacgtaccctgccttggacgtgatgtcacctgggaattcaaccccttgaatgttaaggtcggctcccacatcaccggaggagacttgtacggtatcgtacacgagaacacattggttaagcataagatgttgatcccacccaaggccaagggtaccgtcacctacatcgcgccctccggcaactacaaagtcactgacgtagtgttggagacggagttcgacggcgagaaggagaagtacaccatgttgcaagtatggccggtgcgccagccccgccccgtcactgagaagctgcccgccaaccaccccctgctcaccggacagagagtgctcgactctctcttcccttgtgtccagggtggtaccacggccatccccggcgccttcggttgtggcaagactgtcgtctcacaggctctgtccaagtactccaactctgacgtcatcatctacgtcggatgcggtgaacgtggtaacgagatgtctgaggtactgcgtgacctccccgagctgacggtggagatcgagggcatgaccgagtccatcatgaagcgtaccgcgctcgtcgccaacacctccaacatgcctgtagccgcccgagaggcttccatctacaccggtatcaccctctccgagtacttccgtgatatgggttacaacgtgtccatgatggctgactccacctctcgttgggccgaagctcttcgtgagatctcaggtcgtctggctgagatgcctgccgactccggttaccccgcctacctgggagcccgtctggcctccttctacgagcgtgccggacgtgtgaagtgcctgggtaaccccgacagggagggctccgtgtccatcgtgggcgccgtgtcgccgcccggaggtgacttctccgaccccgtgacggccgccacgctgggtatcgtgcaggtgttctggggt

SEQ ID NO: 139 - DNA, 2724 bp, Spodoptera frugiperda
aactatgaaacacattcgcagatcaagagattcgaggtatgcgacctaccggtgcgcgccgctaagttcgtgcctcgcaagaactgggtgattactggctccgatgacatgcagattcgcgtgttcaactacaacacgcttgagagagtgcacgcctttgaggcgcattctgactacgtcaggtgcatcgcgatacatcccacacagccatacatcctcaccagcagcgatgacctattgatcaagctgtggaactgggaacgcaactgggcatgccagcaagtgttcgagggccacacacattatgtgatgcagatcgtcatcaaccctaaagacaacaacacattcgctagtgctagtctcgacaccaccgtcaaagtatggcagcttggctcttcaatttccaacttcacattagaaggccacgagaaaggcgtgaactgcgtcgactactaccacggcggcgacaagccttacctcataagtggtgccgacgatcgcctcgtcaaaatatgggactaccagaataaaacatgtgtccagacattggagagtcacacgcagaatgtgacagccgtgtcgttccacccggagctgccgatcctgatgactggctcagaggacggcaccatcagaatatggcacgcaggcacttacagacttgaatcctcgctcaactatggcttcgagagagtctggaccatttcctccatgcacggctctaacaacgtagctattggttacgacgagggcacgatcatgatcaaggtgggccgcgaagagccggccatttccatggacgtcaatggaggcaagattatttgggcgaagcattctgagatgcagcaagtcaacctgaaggcgttgccagaaggcacagagataaaagatggagagcgggttccagttatggcgaaggacatgggatcctgtgagatttatccacaaactatagcacacaacccgaacggtcggttcgtggtagtgtgcggggacggcgagtacattatttacacggccatggcacttaggaacaaggcgttcggcactgcacaggagttcgtctgggcgtttgatagctcagagtatgcgacacttgagaactccagcaccatcaaggtgttcaagaacttcaaagagagaaagagtttcaagcctgagtatggtgctgaaggaatatacggcggtttcatgttgggcgtgaagtccatcagcggtgtggcgttctccttctatgattgggagaacttggagttgatcagacggattgagatccagccgcggcacgtgtactggtcggagagcggcaacctggtgtgcctggcggctgatgactcgtactacgtgctcaagtataatgcagctgttgtgacgcgagctcgcgaaaccaactccaacatcacagaagacggcatcgaagacgcttttgaggtcgtgggtgcagtgaacgaggtggtaaagacaggactatgggtgggcgactgcttcatctacacgaattccttgaacagaataaactactacgtcggcggagagatcgtcaccatatcccacctggaccacaccatgtacatcctcggatacgtcgctaaggagaacagactgtacctaaatgacaaggagttgaacatagtgtcgtactcgctgctgctgtcggtactggagtaccagacggcggtgatgcgcgcagacttcgagacagcagaccgggtgctccccaccataccgcaagagcatcgcaccagagtcgctcacttcctagagaaacaaggcttcaaacaacaagctctagcagtctctacggagcctgaacaccagttcgagctggctctctcactgggagaacttagaagagcaaagcagttggccgaggaggcggcattggccgagggttccacctcccgatcctcagcggcacggtggtctcggctgggagcggccgccgccgccgctgcagacacggaactcaccaaggcctgctatcagagcgctaaggactacagtgccctccttctattcgcagctagtactggtgacaaagaactgttgaagagtgtagcacagatgtcctcggaggaaaacgcggagaatatctcattcgtggcctactttatgctgaatgatctcgaatcctgtctgaagctgctcatacagcgggacaagctgcctgaagccgccttctttgccagatcatacattccatcgaaaatgtcggaagtggtgaagttatggcgcgagtccaccagcgccaccaacacgaagctcggccagtcgctcgccgacccggagcagtatgagaatttgttccccgagtttgcgcaatcattggagttggagagatttcagcgcgagtatgggtacgagcagagttgcttgctagcgaacttgccggtcgactcgaagacttgcaaccttgagcgtaaccttgccaccgagaaggaggaggccgagcaacgcggctttaagctgagctccgctgcagtggcgcggtctcctgcacacagctcaaatgatcttggtcctgacgaccgagtcacaaacaatgatagtccttcccacccggtgccggagagcacggtggcgaccgcggcgcagcaggatctgaagaaacggcgcgactctctcgacatcatggaggaactggagcgcgagatcgaagacatcgtcctggacggcacggtcgagagcgtggatctgtcggacgatgtagatttcatggactaa

SEQ ID NO: 140 - DNA, 2838 bp, Spodoptera frugiperda
atgtgtataagagacagctatactctgatcaacttcccgactgattcagagccttacaatgagatgcagctcaagctggatcttgagaaaggygacacaaagaagaaaatagaagcattaaagaaggtaataggtatyatcytktctggkgagaagatwccyggtctattgatgatcatcatccgattygtgctgcccctgcaagaccacaccatcaagaagctgttgctkatcttctgggaaatagtgccaaagaccactcctgatggcaagctcatgcaggagatgatccttgtctgtgatgcttacagaaaggatctgcaacaccccaatgagttcatccgtggctccaccctgcgcttcctctgtaaactgaaggagcctgaactcctcgagcctttgatgcccgccatcagagcttgtcttgaccaccgccactcatatgtcaggaggaatgctgttcttgccatatttactatctacaggaactttgaattcctgatccctgatgctccagagctagtagcaaacttcttggagacagagcaggacatgtcctgcaagaggaatgcatttcttatgctgctccatgctgaccaggagcgggcactgtcctacctctcatccaggctggataatgtacagggctttggagatatccttcagttggttattgtagaacttatttacaaggtttgccacgcgaatccatcagagaggtctcgtttcatccgtacagtatacggcttactgaacgcgaccagtgccgccgtgcggtacgaggctgccggtactctcgttacactgtctaacgcgcctgctgctatcaaggcggcggcagcatgctacatcgacctaatagtgaaggagagcgacaacaacgtgaagctgatagtggtgggtcggctgggagcgctgcgcgcggaggcgggcgaggcggcggcgcgcgcgctgcccgagctggccatggacgtgctgcgcgtgctggccgcctccgacctggacgtgcgccgacacacgctggcgctggcgctcgacctcgtgtcctcccgccacgccgaggacctcgtgcaggtgctgcggaaggaggccgcccgggctaccaacgcagaccacgatgatgctgccaagtacaggcagctacttgttagagctctgcaccgagctgcactcaagttccccgaagtagccggcagtgtagccccggcgctgctggagttgctgggcgacggcagtgagccggccgcgcaggatgtcatgctgttcctacggtccgctctacataccttcgtgacctacggatcatatctaccagaaactgttggaggcggtgcccggtatcaaagtggtaagatagcgcggtctgcgctgtggctgctggcccagttcgctgagactccggaacgcgccaaggatgccttggatgtactcgccaacgtcataccttcccttagcggacaagaggataaggaagaatccgagtcggcagctaaggcccaggacacttcagctccacgacagcttgtcaccagtgatggaacttatgcttcgcagtctgcttttaacttgccagttagccaagcggctccaacccacgcgggtctatgggcggcactaggcgagggcgagagcttcacggcggcgtgcgcgtgctcggcgctgtgcaagctggcgctgaagctgtcgggccgggccgcgaccgccgcgctgcagctggccgcgcgcctgctggccgcccacaagctggccgccggcctcaccgccgacgacgccgagcacggcgcgcgatgtatactggccgcgagacaccgcccgcccgtcgtacaggaagcgctgctgcaacgctcctctgctgcgctcgccgcgctgctagcgctgcctgaccgagctactaatctgcttgatgatgctgataaggaacgtgagccaaagaagcaagacaacaaggtggaggtggaacaaggcatcgtgttcgcccagttggctgggaactccgccgcctccacacaccacgacatgttcgagctatcgctcactaaggcactgcaaggccgcagtaccggcgtgagcgaggagcgcggcaagctgtggaaggtgacgcagctgaccgggttctccgaccccgtgtacgccgaggccatcgtcgccgtcaaccagtacgacatcgtgctcgacgtactcgtcgtcaaccagaccgacgacacgctgcagaactgctgcgtggagctggcgacgctgggcgacctgcggctggtggagcggcccggggccgtggtgctggcccccagggactacgccaccatcaaggcgcacgtcaaggtcgcctccaccgagaacggcatcatcttcgggaacattgtgtacgaggtatcgggcgcgtcgatggaccgcggcgtggtggtcctgaacgacatccacatcgacatcgtggactacatccagcccgccgtctgcaccgacgcagacttcaggcagatgtgggccgagttcgagtgggagaataaggtgtccgtgaacacgaacatcacggacctgcgcgagtacttacaacatttactagcttccacaaacatgaagtgtttgacgcctgaaaaggcattatcgggtcaatgcgggttcatggcagccaacctgtacgcgcggtccatattcggcgaggacgcgctggccaacctgagcatcgagaccccgctgcacaaacccaactcgcccgtcgtcggacatgtcaggatcagggccaagagccagggtatggcgctgtctctaggcgacaaaatcaacatgatgcacaagacgccgcaacagaagaccccctccaaccccatccccgccgcgtaa

SEQ ID NO: 141 - RNA, 79 nt, Artificial Sequence
  Divalent vATPa_545/303
gguaucguacucccuuguguuucaaguacacaagggaagagagagucgauucaaguuguguucucguguacgauaccuu

SEQ ID NO: 142 - RNA, 77 nt, Artificial Sequence
  DIvalent vATPa_542/388
cggcaacuacucuucccuuguucaagucaagggaagagagagucgagguucaagucagugacuuuguaguugccguu

SEQ ID NO: 143 - RNA, 77 nt, Artificial Sequence
  Divalent vATPa_320/501
aagcugcccgagcauaagauuucaaguaucuuaugcuuaaccaaucuuucaaguggggugguuggcgggcagcuuuu

SEQ ID NO: 144 - RNA, 79 nt, Artificial Sequence
  Trivalent COPI_sub_beta_521/731/216
agucccauauuuugacgagguuucaaguacguuguuagagccgugcaucuucaagugauguauggcugugugggauuuu

SEQ ID NO: 145 - RNA, 77 nt, Artificial Sequence
  Divalent COPI_SB_1419/1240
uggcguucuccggcaucgaacucaaguuucgaugccgucuucugugauuucaaguaucauagaaggagaacgccauu

SEQ ID NO: 146 - RNA, 76 nt, Artificial Sequence
  Divalent COPI_SB_1241/1108
ugcgacacuuucuaugauuuucaaguaaucauagaaggagaacgccauucaaguuggaguucucaagugucgcauu

SEQ ID NO: 147 - RNA, 79 nt, Artificial Sequence
  Divalent COPI_BP_1525/1323
ucauaucuaccgacagcuuguucaagucaagcugucguggagcugauguucaagucaacaguuucugguagauaugauu

SEQ ID NO: 148 - RNA, 78 nt, Artificial Sequence
  Divalent COPI_BP_1532/1446
uguauucgccuugucaccauucaaguuggugacaagcugucguggcuuucaaguagguaugacguuggcgaguacauu

SEQ ID NO: 149 - RNA, 77 nt, Artificial Sequence
  Divalent COPI_BP_1448/1490
ggaagaauccucauaccuucuucaagugaagguaugacguuggcgacuucaagugcugccgacucggauucuuccuu

SEQ ID NO: 150 - RNA, 699 nt, Artificial Sequence
  Linked set
  misc_feature (1)..(79): Divalent vATPa_545/303
  misc_feature (80)..(156): Divalent vATPa_320/501
  misc_feature (157)..(233): Divalent vATPa_542/388
  misc_feature (313)..(389): Divalent COPI_SB_1419/1240
  misc_feature (390)..(465): Divalent COPI_SB_1241/1108
  misc_feature (466)..(544): Divalent COPI_BP_1525/1323
  misc_feature (545)..(621): Divalent COPI_BP_1448/1490
  misc_feature (622)..(699): Divalent COPI_BP_1532/1446
gguaucguacucccuuguguuucaaguacacaagggaagagagagucgauucaaguuguguucucguguacgauaccuuaagcugcccgagcauaagauuucaaguaucuuaugcuuaaccaaucuuucaaguggggugguuggcgggcagcuuuucggcaacuacucuucccuuguucaagucaagggaagagagagucgagguucaagucagugacuuuguaguugccguuagucccauauuuugacgagguuucaaguacguuguuagagccgugcaucuucaagugauguauggcugugugggauuaauggcguucuccggcaucgaacucaaguuucgaugccgucuucugugauuucaaguaucauagaaggagaacgccauuugcgacacuuucuaugauuuucaaguaaucauagaaggagaacgccauucaaguuggaguucucaagugucgcauuucauaucuaccgacagcuuguucaagucaagcugucguggagcugauguucaagucaacaguuucugguagauaugauuggaagaauccucauaccuucuucaagugaagguaugacguuggcgacuucaagugcugccgacucggauucuuccuuuguauucgccuugucaccauucaaguuggugacaagcugucguggcuuucaaguagguaugacguuggcgaguacauu

SEQ ID NO: 151 - RNA, 31 nt, Artificial Sequence
  BIV-TAR
gaggcggugaacuuggaaucccacaagggcg

(19) United States
(12) Patent Application Publication
(10) Pub. No.: US 2023/0151370 A1
(43) Pub. Date: May 18, 2023

(54) METHODS AND COMPOSITIONS FOR APTAMER-DRIVEN SURFACE FORMULATION OF SELF-FORMING POLYNUCLEOTIDE NANOPARTICLES

(71) Applicant: HALO-BIO RNAI THERAPEUTICS, INC., Seattle, WA (US)

(72) Inventor: Todd M. HAUSER, Seattle, WA (US)

(21) Appl. No.: 16/642,244

(22) PCT Filed: Jul. 16, 2018
(86) PCT No.: PCT/US2018/042356

§ 371 Date: Feb. 26, 2020

Related U.S. Application Data
(60) Provisional application No. 62/532,913, filed on Jul. 14, 2017.

(51) Int. Cl. C12N 15/115; C12N 15/113; A01N 63/60; A01N 25/26; C12N 15/11

(52) U.S. Cl. CPC C12N 15/115; C12N 15/113; A01N 63/60; A01N 25/26; C12N 15/111; C12N 2310/531; C12N 2310/14; C12N 2310/51; C12N 2310/16; C12N 2310/3519

(57) ABSTRACT

The present invention is directed to compositions and methods for the aptamer-driven surface formulation of self-forming polynucleotide nanoparticles, and the use of such moiety-coated nanoparticle complexes for use in a variety of organisms.

### CROSS-REFERENCE TO RELATED APPLICATION

This application claims the benefit of U.S. Provisional Application No. 62/532,913, filed on Jul. 14, 2017, which is hereby incorporated by reference in its entirety. In cases in which a document incorporated by reference herein is inconsistent with contents of this application, the contents of this application control.

### BACKGROUND OF THE INVENTION

### Field of the Invention

The present invention relates generally to compositions and methods for controlling the surface properties of polynucleotide nanoparticle cores by forming a shell-like surface derived from the binding of select moieties. More specifically, the invention presents methods and compositions for utilizing a plurality of aptamer or intramer sequences within a single-stranded polynucleotide that is self-forming into a compact spherical or discus-like core nanoparticle wherein the aptamer(s) and/or intramer(s) selectively recruit specific organic or inorganic moieties onto the surface of the said polynucleotide nanoparticle core. The resulting moiety-coated nanoparticle core exhibits any mix of altered surface properties of; charge, size, hydrophobicity, and any mix of altered functional properties of; stability, cellular uptake, cellular mobility, cellular recognition, or mode of action, over self-forming polynucleotide nanoparticle cores alone. The aptamer-driven surface formulation methods of this invention enable self-forming polynucleotide nanoparticle cores that are controllably formulated intracellularly, or extracellularly, or in-vitro without the use of canonical coat proteins.

### Description of the Related Art

The safe and effective delivery of polynucleotides to target cells remains a major hurdle that is limiting the potential of polynucleotide-based medicine and agricultural products. A diverse array of evolutionary barriers exist across organisms ranging from degradation challenges, immune recognition, cell specificity, and crossing lipid bilayers once a payload has reached a cell.

Over the last three decades, a large number of solutions to improve the non-viral delivery of polynucleotides have been developed by researchers in both the medical and the agricultural fields—with the medical field primarily leading the way. Aside from chemical modifications, most non-viral delivery methods can be summarized as encapsulated or non-encapsulated, and lipid or non-lipid carriers.

The leading encapsulated solution, and one of the most studied, is a lipid wrapped delivery vehicle currently referred to as Lipid Nanoparticles (LNP). LNPs have been reported to successfully deliver effective polynucleotide payloads to a number of different cells such as human liver cells, solid tumor cells and phagocytic cells. Additional uses in animals, insects, and plants have also been reported with variable results.

The LNP patent portfolio includes U.S. Pat. Nos. 7,745,651, 7,799,565, 8,058,069 and 7,901,708. The '651 patent teaches and claims cationic nitrogen-containing lipids having one or two lineoyl groups that can be used to encapsulate siRNA liposome nanoparticles—increasing their “fusogenicity,” or the ability of the nanoparticles to fuse with cell membranes.

The '565 patent teaches serum-stable nucleic acid-lipid particles (“SNALPs”) that encapsulate interfering RNA, and deliver it into cells. The example SNALP is comprised of an interfering RNA, a non-cationic lipid, a cationic nitrogen-containing lipid, and a bilayer-stabilizing component such as a conjugated lipid, or a polyethylene glycol (“PEG”)-lipid conjugate.

Similar to the '565 patent, the '069 patent describes and also claims serum-stable nucleic acid-lipid particles. Such '069 particles comprise a nucleic acid, a cationic lipid, a non-cationic lipid comprised of phospholipid and cholesterol or cholesterol derivatives, and a conjugated lipid. The '708 patent claims a process for producing lipid vesicles that encapsulate therapeutic agents by mixing an aqueous solution of nucleic acids from one reservoir with an organic lipid solution from a second reservoir to produce a lipid vesicle instantaneously.

While this approach is the current gold standard in exogenous formulation of polynucleotides, it lacks the total surface composition control, supplemental of modes of action, cell specificity, and organism specificity offered by the features of this invention. Most importantly, it is not capable of the intracellular formulation of polynucleotide nanoparticle surfaces offered by this invention.

Additional encapsulating solutions involve the formation of aggregate polyplexes. A variety of materials (i.e., cationic lipids, polymers: natural and synthetic and peptides) have been utilized to fabricate non-viral delivery systems [POLY.1] which have several advantages in terms of safety, ease of preparation, reproducibility, ability to carry large nucleic acid constructs and stability [POLY.2]. Unfortunately, cationic lipids and high molecular weight cationic polymers for gene delivery may cause toxic effect in vitro and in vivo. For example lipoplexes caused several changes to cells, which included cell shrinking, reduced number of mitoses and vacuolization of the cytoplasm [POLY.3]. Cationic polymers, viz., polyethylenimine (PEI), polyamidoamine (PAMAM), polypropylenimine (PPI), poly-L-lysine (PLL), cationic dextran, polyallylamine (PAA), dextran-oligoamine based conjugates and chitosan, [POLY.4], are amongst the preferable materials for the preparation of non-viral vectors in terms of their long-term safety and biocompatibility. PLL, PAA and many others were abandoned due to its low transfection efficiency and higher cytotoxicity. Dextran-oligoamine based transfection in a wide range of cell lines is very low in comparison to other cationic vectors based on PEI, dendrimers etc. Of these, PEI is one of the most successful and widely studied gene delivery polymers due to its membrane destabilization potential, high charge density (nucleotide condensation capability) and ability to protect nucleotides from enzymatic degradation, thus perform nucleotide transfer efficiently into the cells [POLY.5]. Branched PEI contains primary, secondary and tertiary amines in a ratio of 1:2:1 with pKa values spanning around the physiological pH, providing remarkable buffering capacity. Though high charge density of the system increases the transfection efficiency, it simultaneously contributes to increased cytotoxicity.

Chitosan based polyplexes have emerged as a promising candidate for non-viral polynucleotide delivery because of biocompatibility, biodegradability, favorable physicochemical properties and ease of chemical modification. Similar to PEI, the presence of positive charges from amine groups makes chitosan suitable for modification of its physicochemical and biological properties, and enables it to transport the polynucleotides into cells via endocytosis and membrane destability. Most studies to date have shown that high molecular weight (100^{˜}400 kDa), and mid-molecular weight (˜50 kDa) chitosan exhibits aggregation, low solubility under physiological conditions, high viscosity at concentration used for in vivo delivery and slow dissociation or degradation. However, chitosans less than 10 kDa, also known as oligo-chitosans have been described to form weak complexes with polynucleotides, resulting in physically unstable polyplexes with low transfection efficiency.

Chitosan based nanoparticles have been shown to be somewhat effective in the delivery of polynucleotides in plants, insects, animals, and humans.

However, regardless of the type of cationic material used in forming a aggregation-based polyplexes, neither offer the advantages of a controlled surface moiety orientation, surface moiety composition, reduced Nitrogen/Phosphate ratio, or the intracellular surface formulation offered in this invention.

Spherical Nucleic Acid (SNA) nanoparticle conjugates have also been published recently [POLY.6] showing conjugated siRNA arranged spherically around a gold particle. Gold nanoparticles offer both covalent and non-covalent attachment of the active nucleic acid molecule. The arrangement is stacked around the gold particle center. While the approach has proven to be active due to the spherical arrangement of the nucleic acids and cellular penetration, it remains a synthetic (inorganic) delivery vector, and does not have control over surface composition.

MV-RNA polynucleotide nanoparticles were recently shown to be self-forming and an effective of trigger gene silencing [Hauser, PCT/US2016/048492]. Such MV-RNA polynucleotide nanoparticles successfully serve as both the active ingredient and the spherical structural scaffold. Hauser demonstrates the use of aptamers to target certain cells by ligand mediated endocytosis, as well encapsulation by viral coat proteins.

However, the compositions and methods of this invention expand greatly the use of aptamers or intramers in a manner unanticipated by PCT/US2016/048492; resulting in a new paradigm for nanoparticle surface formulation. With the addition of the features of polynucleotide nanoparticles of this invention, a first-of-it's-kind intracellular formulation is possible. Additionally, this invention offers control of nanoparticle surface charge, polarity, surface composition, hydrophobicity, stability, modes of action, cell specificity, cellular recognition, and additional cellular uptake routes over PCT/US2016/048492; without encapsulation by viral coat proteins.

Viral coat proteins or capsid proteins function in the transportation and protection of nucleic acids. It was shown half a century ago that infective virus particles of helical symmetry self-assemble upon mixing aqueous solutions of the coat protein and RNA [VLP.1]. In most cases, this protective layer is due to the presence of multiple copies of a coat protein that self-assemble into what is typically rod or sphere-like shapes surrounding the nucleic acid. While many of the details surrounding the spontaneous self-assembly process remain obscure, recent data suggests that at least the protein-protein interactions and the nucleic acids characteristics dictate the structural outcome. In the case of MS2 VLP's, assembly of coat proteins typically require a short stem-loop RNA hairpin to initiate the packing, which is typically part of their genomic RNA, that leads to subsequent coat proteins assembling into capsids [VLP.3]. In the case of Cowpea Chlorotic Mottle Virus (CCMV), evidence suggests that the diameter is controlled by nucleotide length. Researchers determined that a length of less than 3000 nt resulted in a ˜24-26 nm Coat Protein (CP) diameter and that a length greater than 4,500 nt resulted in a ˜30 nm Coat Protein (CP) diameter when combined with a protein/RNA mass ratio of 6:1.

While the use of CP in-vitro and in-vivo has been demonstrated to encapsulate nucleic acids in VLP's, this RNA length to CP dependency is inefficient for long dsRNA uses and not possible for short RNAi triggers without pre-packaging (i.e., lipids) or encapsulation.

Additionally, CP are a limited group of structural proteins which often stimulate immune responses, and don't solve diverse needs of effective nanoparticle surface formulation beyond that of the CP properties themselves.

Antimicrobial peptides and proteins (AMPs) are a ubiquitous class of naturally occurring molecules that are part of immune response in multicellular organisms. Both Insects and plants primarily produce AMPs to protect against pathogenic invasion. Collectively, the antimicrobial peptides display direct microbicidal activities toward Gram-positive and Gram-negative bacteria, fungi [AMP.13-19], some protozoan parasites [AMP.20] and viruses [AMP.21]. The plant defensins, is a group of small AMPs (45-54 amino acids), highly basic cysteine-rich peptides that are apparently ubiquitous throughout the plant kingdom and display antibacterial and antifungal activities. To date, sequences of more than 80 different plant defensin genes from different plant species are available [AMP.36,AMP.53-56] and isolation of these has been recently patented (U.S. Pat. Nos. 6,911,577, 6,770,750 and EP1849868). Consistent with a defensive role, they are particularly abundant in seeds, but have also been described in leaves, pods, tubers, fruit and floral tissues [AMP.17,57].

More than 7000 naturally occurring peptides have been identified, and these often have crucial roles in human physiology, including actions as hormones, neurotransmitters, growth factors, ion channel ligands, or anti-infectives [THER.1-THER.4]. Peptides are recognized for being highly selective and efficacious and, at the same time, relatively safe and well tolerated. Consequently, there is an increased interest in peptides in pharmaceutical research and development (R&D), and approximately 140 peptide therapeutics are currently being evaluated in clinical trials. However, naturally occurring peptides are often not directly suitable for use as convenient therapeutics because they have intrinsic weaknesses, including poor chemical and physical stability, and a short circulating plasma half-life.

While it is clear that peptides are a significant part of immune defense across many organisms, and useful in medical and agriculture uses. The use of surface-bound peptides has not been shown in the art as an intracellular nanoparticle coating moiety—nor has a controlled binding of naturally occurring compounds onto the surface geometry of a nanoparticle been studied as a means to supplement the activity and bioavailability this ancient immune system.

Despite decades of developments in nanoparticle delivery, and the use of peptides, proteins, and aptamers as biomolecules, there remains a need for methods and compositions that allow for polynucleotide core nanoparticles with controlled surface features affecting; size, charge, composition, hydrophobicity, nuclease stability, modes of action, cell specificity, cell uptake, and overall bioavailability—that can be produced in-vitro, or extracellular, or intracellularly. The present invention addresses this need, and has novel uses in humans, animals, plants, insects, bacteria, and fungus.

### BRIEF SUMMARY OF THE INVENTION

The aptamer-driven surface formulation methods of this invention provide novel compositions useful in the delivery of such coated polynucleotide nanoparticles by enabling compositional control of functional and non-functional surface characteristics within a range of environments. Such aptamer-driven surface formulation method of this invention enable compositional control of surface-bound moieties such as; peptides, pre-cursor protein, proteins, polymers, metabolites, ions, small molecules, oligosaccharides, or other organic or inorganic moieties in either an in-vitro, or extracellular, or intracellular setting.

The aptamer-driven surface formulation methods of this invention combine particular polynucleotide nanostructure with a plurality of single or multivalent aptamer/intramers in a manner leading to novel nanoparticle surface formulation. This new aptamer/intramer-driven surface formulation method enables the creation of coated biomolecule complexes with novel advantages over isolated polynucleotide nanoparticles, aptamers, peptides, or proteins in a diverse range of settings.

The aptamer-driven surface formulation methods of this invention enable control of surface characteristics affecting particle diameter, surface zeta potential, hydrophobicity, function, cellular uptake, organism specificity, cellular specificity, nuclease degradation resistance, receptor recognition, translocation, pharmaceutical index, toxicity, and even mode of activity—beyond that of the polynucleotide nanoparticles not using this invention (FIG. 8).

The aptamer-driven surface formulation methods of this invention are distinguished from other formulation methods due the nanoparticles self-forming spherical structure utilized as the core scaffold [Hauser, PCT/US2016/048492]. This spherical core requires only a minimal moiety coating to significantly change surface and performance characteristics, thus enabling an aptamer/intramer-driven formulation method. In contrast, most nanoparticle formulation methods form nanoparticles by aggregation—which requires larger and significantly more components. Comparatively, an exponentially lower number of surface units are required to alter the surface characteristics of this invention. For example, the Nitrogen/Phosphate ratios of this invention are orders of magnitude lower than that of typical siRNA formulation techniques [POLY.8, FIG. 16]. Additionally, the pre-formed aptamer-driven surface formulation method cores of this invention enable the use of low molecular-weight linear substrates—which are less cytotoxic than the larger non-linear equivalents [POLY.8], and enables the use of vast types of small organic or inorganic moieties as new coating substrates.

This invention is further distinguished by the arrangement and orientation of a plurality of single or multivalent aptamer(s) and/or intramer(s) within the self-forming single stranded polynucleotide which orient the aptamers and/or intramer(s) on the surface of the nanoparticle and specifically recruit by binding non-covalently to select moieties onto the polynucleotide surface in a desired composition and molarity. Such arrangements allow for the programmable composition of surface moieties to control surface regions of function, charge, hydrophobicity, etc.; (i.e. mimicking other particle surface properties (FIG. 10)).

Importantly, this invention enables the recruitment of surface moieties that are not solely dependent upon non-specific and/or weaker electrostatic binding typical of free moieties and isolated aptamer/intramers at biological pH. The application of this invention enables specific moiety binding and higher long-range binding forces due to the single and multivalent intramer(s)/aptamer(s) oriented on the surface of compact, highly-anionic polynucleotide nanoparticle core.

This invention is additionally distinguished by the orientation of a plurality of aptamer(s) or intramer(s) within the self-forming single stranded polynucleotide which orient the aptamer(s) and/or intramer(s) on the surface of the said nanoparticle and selectively recruit by binding non-covalently to specific neutral, anionic or cationic moieties onto the polynucleotide surface at a physiological pH.

This invention provides a unique combination of features within an isolated polynucleotide nanoparticle core that has a self-forming sphere-like diameter and also surface-oriented aptamer(s) and/or intramer(s) partially or fully dedicated to recruiting specific moieties onto the nanoparticles surface. This results an extremely low number of surface moieties required in order to change the functional and/or non-functional surface characteristics of the polynucleotide nanoparticle core alone. The combination of features of this invention enable a first-of-its-kind intracellular auto-formulation method leading to new capabilities in agriculture and medicine.

In certain embodiments, the polynucleotide nanoparticle core is composed of 2, 3, 6, 9, 12, 15, 27 or more separate MV-RNA, siRNA, RNA or DNA hairpin molecules joined by linkage nucleotides into a single-stranded self-forming polynucleotide disc-like or sphere-like nanoparticle structure.

In preferred embodiments, a surface-formulating aptamer(s) and/or intramer(s) forms the loop of the MV-RNA, shRNA, miRNA, RNA or DNA hairpin molecules joined by linkage nucleotides into a single-stranded self-forming polynucleotide disc-like or sphere-like nanoparticle structure.

In other preferred embodiments, a surface-formulating aptamer(s) and/or intramer(s) hairpin resides in-between each MV-RNA, shRNA, miRNA, RNA or DNA hairpin molecules joined by linkage nucleotides into a single-stranded self-forming polynucleotide disc-like or sphere-like nanoparticle structure.

In certain embodiments, the isolated surface-formulating polynucleotide nanoparticle has a plurality of MV-RNA, shRNA, miRNA, RNA or DNA hairpin molecules in a general structure set forth in any one of (FIG. 2a-c).

In certain embodiments, the isolated surface-formulating polynucleotide nanoparticle determines the polynucleotide diameter of approximately 20 nm, 30 nm, 40 nm, 40-100 nm, 100-200 nm, 200-600 nm, ideally less than 200 nm.

In still other specific embodiments, the polynucleotide nanoparticle comprises natural or synthetic RNA or DNA, preferably RNA.

In still other specific embodiments, the polynucleotide nanoparticle comprises natural or synthetic RNA or DNA, 2′ modified nucleotides, locked or unlocked nucleotides.

According to another aspect of the invention provides composition comprising one or more isolated aptamer-driven surface formulation method, as described in any of the embodiments herein, in combination with a physiologically acceptable excipient.

According to still another aspect of the invention provides methods for delivering two or more RNA molecules to a target cell comprising contacting the target cell with an isolated polynucleotide nanoparticle composition described herein.

In preferred embodiments, the isolated or group of aptamer(s) and/or intramer(s) within the single-stranded polynucleotide nanoparticle are experimentally selected to recruit a surface moiety by binding specifically to a peptide, protein, small molecule, metabolite, organic or inorganic chemical from the results of SELEX, or other Aptamer/moiety binding assays.

In certain embodiments, the isolated or group of randomized aptamer(s) and/or intramer(s) for a given target moiety are transcribed within the single-stranded self-forming polynucleotide nanoparticle used in SELEX, or other aptamer/moiety binding assays.

In other embodiments, the isolated or group of randomized aptamer(s) and/or intramer(s) used in SELEX, or other Aptamer/moiety binding assays, are transcribed individually; then later combined with the polynucleotide nanoparticle sequence to create a final surface-forming polynucleotide nanoparticle.

In certain embodiments, the isolated aptamer(s) and/or intramer(s) within the single-stranded polynucleotide nanoparticle are designed to recruit a surface moiety by binding specifically to moieties intracellularly, extracellularly, in-vitro, in-vivo, or any combination thereof.

In certain embodiments, the isolated aptamer(s) and/or intramer(s) within the single-stranded polynucleotide nanoparticle are designed to recruit a surface moiety by binding specifically to moieties that are endogenous, exogenous, or any combination thereof.

In other specific embodiments, the surface-forming polynucleotide nanoparticle is expressed and surface-formulated within a host cell selected from a human cell or animal cell or plant cell or yeast cell or insect cell or bacterial cell, or by in-vitro transcription.

In certain specific embodiments, the surface-forming polynucleotide nanoparticle is produced by intracellular transcription by a promoter (transgenic), virus (transient), or applied topically (exogenic) following in-vitro transcription in a general structure set forth in any one of FIGS. 1-2, 4.

In certain preferred embodiments, the target surface moiety of the aptamer(s)/intramer(s) is a peptides or protein precursor chosen from the host organisms peptidome.

In certain other embodiments, the target surface moiety of the aptamer(s)/intramer(s) is a peptides or protein precursor chosen from the target organisms peptidome.

In other preferred embodiments, the target surface moiety of the aptamer(s)/intramer(s) is a peptide, protein precursor, or protein transiently or transgenically expressed in the host organism.

In other specific embodiments, the cellular or membrane penetration rate of the moeity-coated polynucleotide nanoparticles of this invention are increased.

In still other embodiments, the endosomal escape rate of the moiety coated polynucleotide nanoparticles of this invention are increased.

In certain embodiments, the isolated polynucleotide nanoparticle core targets genes of insects, or virus, or fungus, or animals, or humans, or host plant (FIG. 8a), other plants, or any combination thereof; and the surface moiety targets insects, or virus, or bacteria, or fungus, or animals, or humans, or host plant, or any combination thereof by using a general structure set forth in any one of (FIG. 8b).

In certain embodiments, additional mode(s) of action of isolated polynucleotide nanoparticle are added upon binding of the target surface moiety (FIG. 3b, 9, 10, 11).

In specific embodiments, the isolated polynucleotide nanoparticle changes the surface charge, nuclease resistance, protease resistance, mode of action, and molecular weight upon binding of the target surface moiety (FIG. 3b, 10, 11).

In other specific embodiments, the surface charge of isolated polynucleotide nanoparticle becomes less anionic, neutral, or cationic upon binding of the target surface moiety (FIG. 3b, 10, 11).

In other specific embodiments, the isolated polynucleotide nanoparticle targets genes in organisms other than those of the host. Organism specificity can be determined by complementarity of the polynucleotides to the target genes and cellular uptake signals such as aptamers, ligands, linkage nucleotides, loops, long dsRNA, ssRNA ends, function of bound surface moieties, or a combination thereof (FIG. 9).

In specific embodiments, the isolated polynucleotide nanoparticle is gene modulating by RNAi, but coating surface moiety is anti-microbial, anti-fungal, or both (FIG. 9, 11b-c).

In specific other embodiments, the isolated polynucleotide nanoparticle is gene modulating by RNAi, but coating surface moiety is toxic protein, peptide, chemical, or combination of.

In other specific embodiments, mode(s) of action of the target surface moiety are decreased upon binding of the target surface moiety to the isolated polynucleotide nanoparticle.

In still other specific embodiments, the isolated polynucleotide nanoparticle is a single polynucleotide nanoparticle coated with Anti-microbial Peptides, Antifungal Peptides, toxic proteins, or combination thereof. (FIGS. 8-11).

In certain embodiments, the moiety-coated polynucleotide nanoparticle enabled by this invention increases the activity of the isolated surface moiety.

In certain embodiments, the moiety-coated polynucleotide nanoparticle enabled by this invention decreases the activity of the isolated surface moiety.

In certain embodiments, the moiety-coated polynucleotide nanoparticle enabled by this invention increases the activity of the core polynucleotide nanoparticle.

In certain embodiments, the moiety-coated polynucleotide nanoparticle enabled by this invention decreases the activity of the core polynucleotide nanoparticle.

### BRIEF DESCRIPTION OF THE DRAWINGS

FIG. 1. FIG. 1a shows an ‘slice view’ example of the self-forming, single-stranded nanoparticle core of PCT/US2016/048492. FIG. 1b shows a ‘slice view’ surface region formed by the self-forming, single-stranded nanoparticle of PCT/US2016/048492 and utilized by this invention. FIG. 1c shows a single surface-oriented, moiety-binding aptamer/intramer as a sub-unit of this invention. FIG. 1d shows a different single surface-oriented, moiety-binding aptamer/intramer as a sub-unit of this invention.

FIG. 2. FIG. 2a shows a ‘slice view’ example of the preferred ˜40 nm diameter of the self-forming, single-stranded nanoparticle core of PCT/US2016/048492. FIG. 2b shows a ‘slice view’ example of the additionally preferred ˜20 nm diameter of an ultra-small self-forming, single-stranded RNAi nanoparticle core contained within this invention. FIG. 2c shows a ‘slice view’ example of the non-preferred ˜40-200 nm diameter of a plurality of canonical dsRNA, shRNA, or miRNA, utilized as a non-inventive nanoparticle core.

FIG. 3. FIG. 3a shows (1) a ‘slice view’ example of the self-forming, single-stranded nanoparticle core of PCT/US2016/048492, (2) an array of aptamer(s)/intramer(s) dedicated to recruiting moieties onto the nanoparticle surface as described in this invention, and (3) the anionic surface of the polynucleotide core nanoparticle with protruding aptamer(s)/intramer(s) before formulation (moiety-binding). FIG. 3b shows (1) a ‘slice view’ example of the self-forming, single-stranded nanoparticle core of PCT/US2016/048492, (2) an array of aptamer(s)/intramer(s) dedicated to recruiting moieties onto the nanoparticle surface as described in this invention, and (3) an example of an altered nanoparticle shell characteristics (charge, size, composition) formed by selective moiety-binding as a result of the aptamer(s)/intramer(s) array of this invention.

FIG. 4. FIG. 4a shows a partial secondary structure example of how the nanoparticle surface is composed by substituting some or all of the polynucleotide nanoparticle core loops (“Loop of”) with the aptamer(s)/intramer(s) sequences. FIG. 4b shows a partial secondary structure example of how the nanoparticle surface is composed by placing a stem-containing aptamer/intramer sequence in-between (“In-between of”) some or all of the polynucleotide nanoparticle core subunits.

FIG. 5. FIG. 5a-d shows a method in which the sequence format described in PCT/US2016/048492 can be combined with grouped surface-forming aptamer(s)/intramer(s) to form (a) aptamer(s)/intramer(s) clusters, and (b) additional aptamer(s)/intramer(s) cluster(s) to recruit a moiety (c) onto a certain surface region, and to recruit a moiety (d) onto a separate surface region. FIG. 5e shows example integration a 3-way junction aptamer(s)/intramer(s), built from the secondary structure of an individual MV-RNA, utilized to increase binding affinity at broader pH range of less cationic moieties and/or increase specificity to select moieties.

FIG. 6 shows a non-limiting intracellular environment containing a nucleus, cytoplasm at biological pH, but could lack a nucleus. FIG. 6a Shows an un-bound, non-protein, moiety contained within the intracellular environment. FIG. 6b Shows an un-bound, protein, moiety contained within the intracellular environment. FIG. 6c Shows an example polynucleotide nanoparticle core with the surface-oriented aptamer(s)/intramer(s) of this invention transcribed within the cell. FIG. 6d Shows an example polynucleotide nanoparticle core with the surface-oriented aptamer(s)/intramer(s) of this invention transcribed within the cell.

FIG. 7 shows how in-vitro formulation of the polynucleotide nanoparticle core complex can be used to selectively formulate a nanoparticle surface across a pH gradient. FIG. 7a shows moiety binding onto surface aptamer(s)/intramer(s) at biological pH. FIG. 7b shows an increase in electrostatic binding strength of moieties onto surface aptamer(s)/intramer(s) at acidic pH ranges.

FIG. 8 shows the increased spectrum of activity from the nanoparticle core shells formed by this invention. FIG. 8a shows the typical spectrum of activity for typical polynucleotide nanoparticle cores. FIG. 8b shows the additional spectrum of activity of a polynucleotide nanoparticle in Bacteria, Archea, Eukarya due to the functionalized shells formed by this invention.

FIG. 9 shows the functional relationship between polynucleotide nanoparticle cores and the shells created by this invention. For each organism, non-limiting examples of functional shell “Coating Material” moieties are listed.

FIG. 10 shows the concept of aptamer(s)/intramer(s) grouping to form recruit clusters of moieties onto the surface of a polynucleotide core in a manner that mimics functional ABE Toxin properties; Activity, Binding, Entry or other grouped surface assemblies. FIG. 10a shows the Domain in which aptamer(s)/intramer(s) are grouped in order to recruit clusters of specific ‘Active’ moieties, i.e., toxic peptides. FIG. 10b shows the Domain in which aptamer(s)/intramer(s) are grouped in order to recruit clusters of specific ‘Binding’ moieties, i.e., receptor binding peptides or proteins. FIG. 10c shows the Domain in which aptamer(s)/intramer(s) are grouped in order to recruit clusters of specific ‘Entry’ moieties, i.e., membrane disrupting, endosomolytic peptides.

FIG. 11 shows non-limiting models of multi-model, functionalized polynucleotide core/shell nanoparticles enabled by this invention. FIG. 11a shows a polynucleotide nanoparticle core targeting genes of an insect, with a functionalized shell formed by the surface aptamer(s)/intramer(s) in functional clusters that recruit endogenous Herbivory Response Peptides, plant proteins, and cell-binding peptides onto the surface of the said polynucleotide nanoparticle core. FIG. 11b shows a polynucleotide nanoparticle core targeting genes of an fungi, with a functionalized shell formed by the surface aptamer(s)/intramer(s) in functional clusters that recruit endogenous Herbivory Response Peptides, anti-fungal peptides, and cell-binding peptides onto the surface of the said polynucleotide nanoparticle core. FIG. 11c shows a polynucleotide nanoparticle core targeting genes of an human, with a functionalized shell formed by the surface aptamer(s)/intramer(s) in functional clusters that recruit endogenous Antibacterial Peptides of one type, Antibacterial peptides of another type, and cell-binding peptides onto the surface of the said polynucleotide nanoparticle core.

FIG. 12 Illustrates efficient developmental stages leading to final aptamer-driven surface compositions and molarity. FIG. 12a Illustrates the use of cationic additives to a candidate surface moiety to cause complexion with the anionic polynucleotide nanoparticle as a limited ‘first step’ in screening potential substances. FIG. 12b Illustrates the use of pseudo-aptamer amino acid sequences added to a candidate surface moiety to cause specific and programmable complexion with the equivalent aptamer(s)/intramer(s) of the polynucleotide nanoparticle as a ‘useful step’ in screening potential substance location, function and molarity. FIG. 12c Illustrates the preferred embodiment of this invention in which specific aptamers are developed for intended surface moieties. FIG. 12d Demonstrates the pattern of correctly folded RNA for candidate polynucleotide nanoparticles as output from co-fold.

FIG. 13 shows the formulation calculations used for the equimolar binding comparisons between ECB-2 and ECB-3 (EXAMPLE 1) in the ITC analysis provide in FIG. 16. FIG. 13a shows the formulation calculation table for the aptamer-driven binding of peptide TAT-NP on ECB-2 of EXAMPLE 1. FIG. 13a shows the formulation calculation table for the aptamer-driven binding of peptide TAT-NP on ECB-3 of EXAMPLE 1.

FIG. 14. FIG. 14a shows the efficient Nitrogen/phosphate (N/P) ratio of self-forming spherical polynucleotide nanoparticle formulation. Gel shift assays provide visual evidence of the anionic polynucleotide nanoparticle transition to cationic at a given molar or N/P ratio. Surface Zeta Potential measurements provide a second measurement of the charge transition of the polynucleotide core nanoparticles at given N/P ratios. Electrostatic based formulation is tested using linear Polyethylenimine 2 kDa, linear Cell-Penetrating Peptide 2 kDa, or Low Molecular Weight Chitosan, 85% deacetylated. FIG. 14b Shows the gel shift assay between FAW-2 of Example 2, using aptamer-driven binding. Additionally, heparin was demonstrated as a successful antagonist of the TAT aptamer binding of the TAT-NPF peptide and disassembled the complex.

FIG. 15 shows the Zeta Sizer measurement of the hydrodynamic shell of a polynucleotide nanoparticle alone, a surface moiety substance alone, and when the two are combined. FIG. 15a shows the Zeta Sizer measurement of the hydrodynamic shell of the FAW-3 (EXAMPLE 2) nanoparticle alone, aggregated TAT-LYCO surface moiety substance alone, and size transition when the two are combined under the same conditions. FIG. 15b shows the Zeta Sizer measurement of the hydrodynamic shell of the FAW-3 (EXAMPLE 2) nanoparticle alone, monomeric CM-TAT surface moiety substance alone, and size transition when the two are combined under the same conditions.

FIG. 16 provides the comparative isothermal titration colorimetry analysis of the electrostatic-driven binding used in the industry vs. the aptamer-driven binding described in this invention as a preferred method for nanoparticle surface formulation. FIG. 16a Provides the output of the equimolar ITC analysis profiling the binding of peptide CM-TAT on ECB-3, a polynucleotide nanoparticle combining this invention with ECB-2. The data represents a 30-300×improvement in binding due to the benefit of the aptamer-driven surface formulations of this invention. FIG. 16a Provides the output of the equimolar ITC analysis profiling the electrostatic-driven binding of peptide CM-TAT on ECB-2, a polynucleotide nanoparticle not using this invention.

FIG. 17 provides binding data using three sets of polynucleotide/peptide relationship; 1) non-specific cationic peptide/nanoparticle with non-specific aptamer, 2) specific cationic peptide/nanoparticle with specific aptamer, 3) specific cationic peptide/non-specific nanoparticle in H₂O and two buffer concentrations. The data suggests increased binding by utilizing the design and methods of this patent.

### DETAILED DESCRIPTION OF THE INVENTION

While the invention has been described with reference to the aforementioned specification, the descriptions and illustrations of the preferable embodiments herein are not meant to be construed in a limiting sense. It shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art upon reference to the present disclosure. It is therefore contemplated that the appended claims shall also cover any such modifications, variations and equivalents.

The practice of various embodiments of the invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)). As used in the specification and claims, the singular form “a”, “an” and “the” include plural references unless the context clearly dictates otherwise. For example, the term “a cell” includes a plurality of cells, including mixtures thereof.

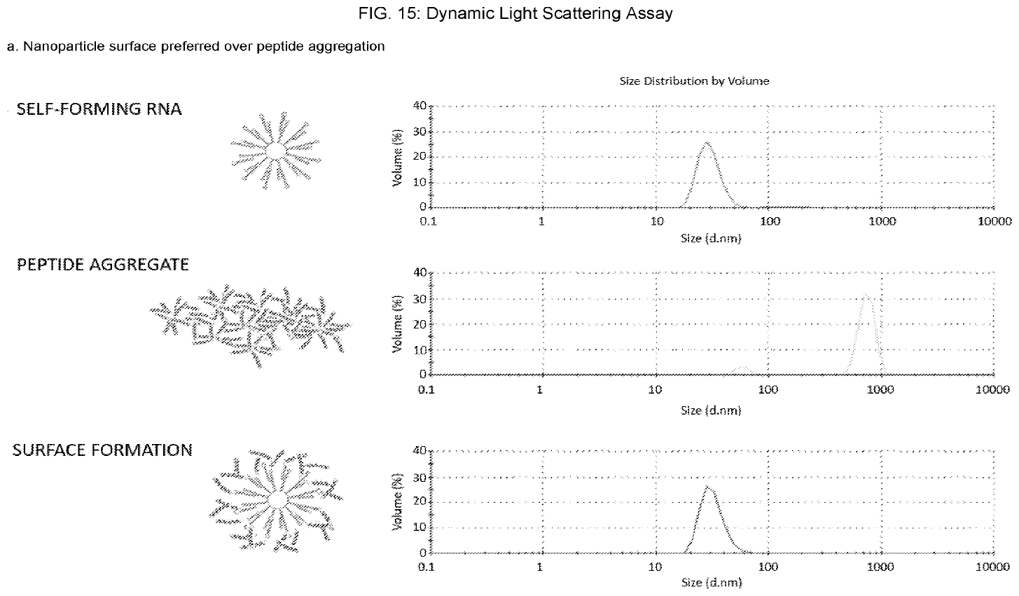

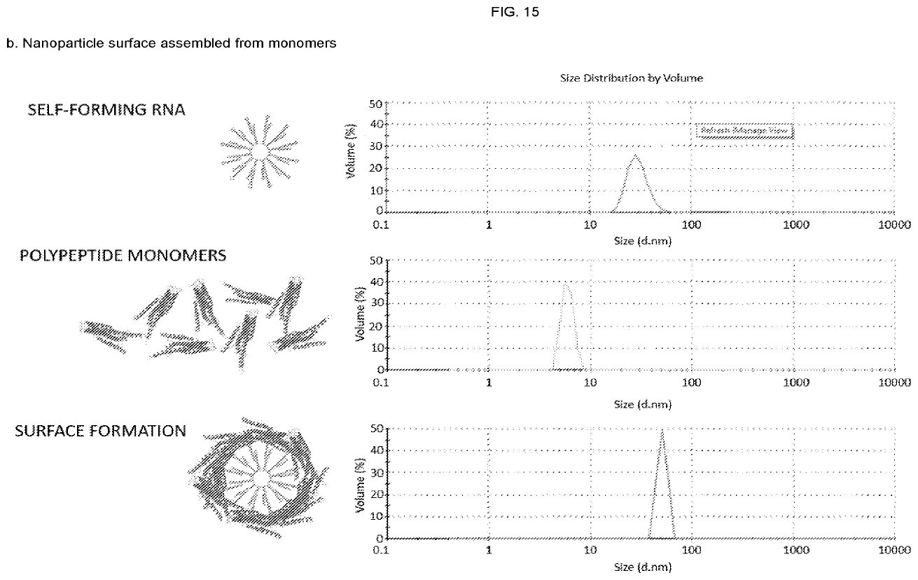

According to the embodiments of the disclosure, self-forming polynucleotide nanoparticles are provided herein. A self-forming polynucleotide nanoparticle includes a polynucleotide core (or polynucleotide nanoparticle core) and a moiety binding region. The polynucleotide core includes one or more multivalent RNA (MV-RNA) molecules connected to each other. Multivalent RNA (MV-RNA) represents a junction-class RNA molecule that is not canonical dsRNA, but which has a similar mode of action to dsRNA-based RNAi molecules described above. Uniquely, MV-RNA exhibits the ability to cleave multiple sites on the same or different genes simultaneously as well as utilize different pre-processing pathway than dsRNAi (U.S. Patent Publication No. 2011/0159586 and PCT Publication No. WO2012/014155) (FIG. 15). Additional embodiments and information regarding polynucleotide core structure that can be used in accordance with the embodiments described herein are described in detail in International Publication No. PCT/US2016/048492 to Hauser, which is incorporated herein in its entirety, as if fully set forth herein.

The MV-RNA molecules that form the polynucleotide nanoparticle core described herein may include one or more aptamers or intramers capable of binding one or more surface moieties within the moiety binding region. In other words, the MV-RNA molecules of the polynucleotide nanoparticle core of the self-forming polynucleotide nanoparticles described herein are designed to have aptamer-driven (or intramer-driven) binding.

The aptamer-driven surface formulation (i.e., design) methods of this invention provide novel compositions and methods useful in the delivery of polynucleotide nanoparticles by enabling compositional control of functional and non-functional surface characteristics within a range of environments. The aptamer-driven surface formulation method of this invention enables compositional control of surface-bound moieties such as; peptides, pre-cursor proteins, proteins, polymers, metabolites, ions, small molecules, oligosaccharides, or other organic or inorganic moieties in either an in-vitro, or extracellular, or intracellular setting.

The aptamer-driven surface formulation methods of this invention combine polynucleotide nanostructures with a plurality of single or multivalent aptamer/intramers in a manner leading to novel nanoparticle surface formulation. This aptamer/intramer-driven surface formulation method enables the creation of coated biomolecule complexes with novel advantages over isolated polynucleotide nanoparticles, aptamers, peptides, or proteins in a diverse range of settings.

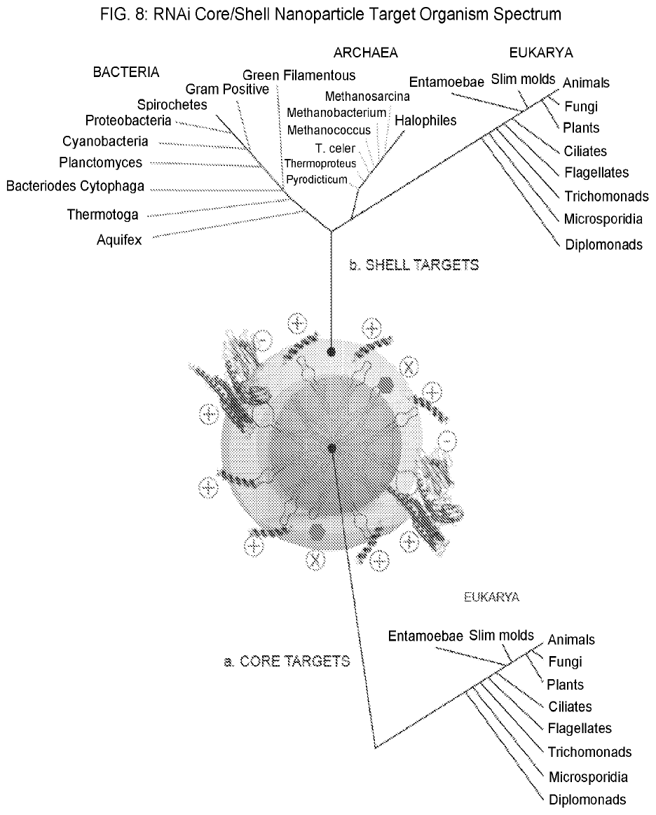

The aptamer-driven surface formulation methods of this invention enable control of surface characteristics affecting particle diameter, surface zeta potential, polarity, hydrophobicity, function, cellular uptake, cellular recognition, organism specificity, cellular specificity, degradation resistance, receptor recognition, translocation, pharmaceutical index, toxicity, and even mode(s) of activity—beyond that of polynucleotide nanoparticles not using this invention (FIG. 8).

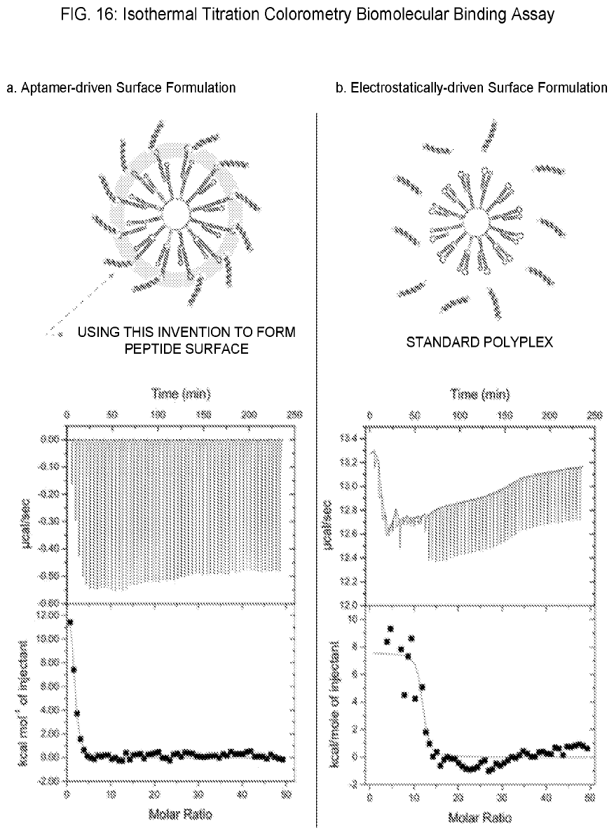

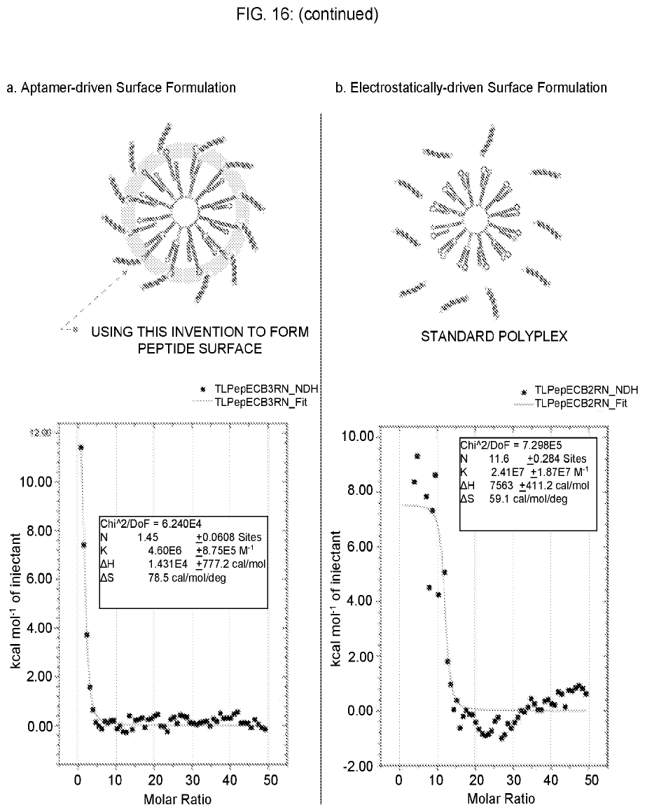

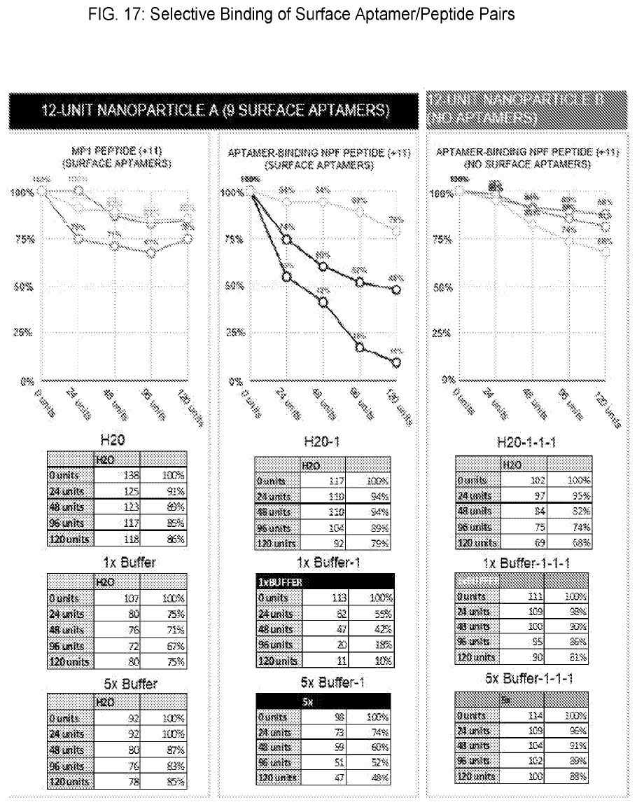

The aptamer-driven surface formulation methods of this invention are distinguished from other formulation methods due the nanoparticles self-forming spherical structure utilized as the core scaffold [Hauser, PCT/US2016/048492]. This ‘pre-formed’ spherical core requires only a minimal moiety coating to significantly change surface and performance characteristics, thus enabling the aptamer/intramer-driven formulation method of this invention. In contrast, most nanoparticle formulation methods (polyplexes) form nanoparticles by aggregation-preferring both non-linear and a higher molarity of cationic material to form useable nanoparticles. Comparatively, an exponentially lower number of surface units are required to alter the surface characteristics of the core scaffold (FIG. 8). For example, the Nitrogen/Phosphate ratios of the surface-forming nanoparticles of this invention are orders of magnitude lower than that of typical siRNA formulation techniques [POLY.8, FIG. 16]. Additionally, the pre-formed aptamer-driven surface formulation method cores of this invention enable the use of low molecular-weight linear substrates—which are less cytotoxic than the larger non-linear equivalents [POLY.8], and enables the use of vast types of small organic or inorganic moieties as new coating substrates that would not be possible using past formulation methods.

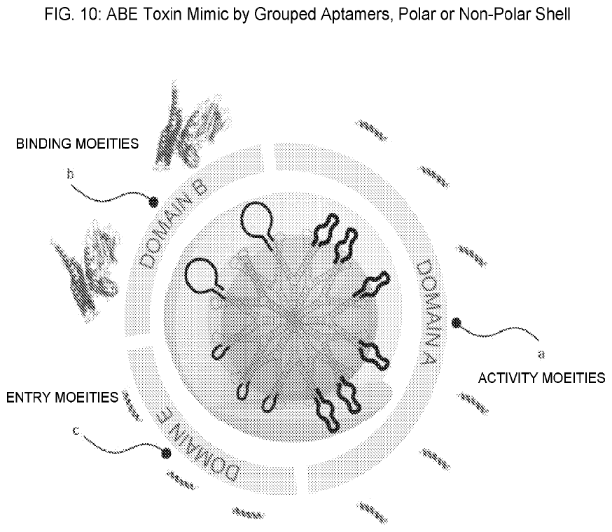

This aptamer-driven surface formulation invention is distinguished by the arrangement and orientation of a plurality of single or multivalent aptamer(s) and/or intramer(s) within the self-forming single stranded polynucleotide which orient the aptamers and/or intramer(s) on the surface of the final nanoparticle, and specifically recruit select moieties by non-covalent aptamer-driven binding onto the polynucleotide surface in a desired composition and molarity. Such arrangements allows for programmable composition of surface moieties to control surface regions of function, charge, hydrophobicity, etc.; (i.e. even able to mimic other particle surface properties (FIG. 10)).

Importantly, this invention enables the recruitment of surface moieties that would otherwise be non-specific, and/or have insufficient electrostatic properties for reliable surface-binding onto nanoparticles not using this invention. Additionally, the methods of this invention enable the development of aptamer/intramer sequences within a self-forming nanostructure that result in binding of free moieties and isolated aptamer/intramers at biological pH. The application of this invention enables specific moiety binding, and higher long-range binding forces due to the single and multivalent intramer(s)/aptamer(s) oriented on the surface of compact, highly-anionic polynucleotide nanoparticle core. Such methods of embedding aptamer/intramer with nanostructure in the SELEX process allows for identification of surface moiety-binding aptamer sequences especially useful in intracellular environments.

This invention is additionally distinguished by the orientation of a plurality of aptamer(s) or intramer(s) within the self-forming single stranded polynucleotide which orient the aptamer(s) and/or intramer(s) on the surface of the said nanoparticle and selectively recruit specific aptamer-targeted neutral, anionic, or cationic moieties onto the polynucleotide surface at a broad pH range, including physiological pH.

This invention provides a unique combination of features within an isolated polynucleotide nanoparticle core that has a self-forming sphere-like diameter, and surface-oriented aptamer(s) and/or intramer(s) partially or fully dedicated to recruiting specific moieties onto the nanoparticles surface. This results an extremely low number of surface moieties required in order to change the functional and/or non-functional surface characteristics of the polynucleotide nanoparticle core alone. The combination of features of this invention enable a first-of-its-kind intracellular auto-formulation method leading to new capabilities in agriculture and medicine.

Nanoparticle Core Compositions

According to some embodiments, the polynucleotide core comprises two or more connected MV-RNA, each separated by one or more nucleotides, resulting in at least one biologically active MV-RNA molecule after endonuclease biogenesis. Each MV-RNA removed from the nanoparticle by Dicer or Dicer-like nuclease cleavage is able to load into downstream silencing complexes, including but not limited to RNA Induced Silencing Complex (RISC) and miRNA-Induced Silencing Complex (miRISC). The removed MV-RNAs may also function in downstream immune-stimulatory events. The possibility for both gene suppression and immune-stimulant characteristics within a single nanoparticle offers the ability to suppress antagonists to immune surveillance in certain cancers while simultaneously stimulating the immune response to that particular cell. In this manner, the polynucleotide nanoparticles provided herein act as a unique single-stranded and purely RNA nanoparticle precursor for RNA Interference, miRNA Interference, or immunotherapy—one that can contain a highly-scalable active trigger molarity.

In certain embodiments, the polynucleotide nanoparticle core is composed of 2, 3, 6, 9, 12, 15, 27 or more separate MV-RNA, siRNA, RNA or DNA hairpin molecules joined by linkage nucleotides into a single-stranded self-forming polynucleotide disc-like or sphere-like nanoparticle structure.

In preferred embodiments, one or more of the MV-RNA, siRNA, RNA or DNA hairpin molecules include an aptamer and/or intramer that forms the loop of each hairpin molecule. In certain aspects, a plurality of the MV-RNA, siRNA, RNA or DNA hairpin molecules include an aptamer and/or intramer that forms the loop of each hairpin molecule. The aptamer and/or intramer can replace the loop region of the hairpin molecule, and can be selected to target a particular surface moiety, or can be randomized for use in selection assays like SELEX, as described further below. The aptamer(s) and/or intramer(s) are therefore capable of targeting and binding surface moieties in the moiety binding region.

In other preferred embodiments, a surface-formulating aptamer(s) and/or intramer(s) forms the loop of the MV-RNA, shRNA, miRNA, RNA or DNA hairpin molecules joined by linkage nucleotides into a single-stranded self-forming polynucleotide disc-like or sphere-like nanoparticle structure.

In other preferred embodiments, a surface-formulating aptamer(s) and/or intramer(s) hairpin resides in-between each MV-RNA, shRNA, miRNA, RNA or DNA hairpin molecules joined by linkage nucleotides into a single-stranded self-forming polynucleotide disc-like or sphere-like nanoparticle structure.

In certain embodiments, the isolated surface-formulating polynucleotide nanoparticle has a plurality of MV-RNA, shRNA, miRNA, RNA or DNA hairpin molecules in a general structure set forth in any one of (FIG. 2a-c).

In certain embodiments, the isolated surface-formulating polynucleotide nanoparticle determines the polynucleotide diameter of approximately 20 nm, 30 nm, 40 nm, 40-100 nm, 100-200 nm, 200-600 nm, ideally less than 200 nm.

In still other specific embodiments, the polynucleotide nanoparticle comprises natural or synthetic RNA or DNA, preferably RNA.

In still other specific embodiments, the polynucleotide nanoparticle comprises natural or synthetic RNA or DNA, 2′ modified nucleotides, locked or unlocked nucleotides.

In certain embodiments, the moiety-coated polynucleotide nanoparticle enabled by this invention decreases the activity of the isolated surface moiety.

In other specific embodiments, mode(s) of action of the target surface moiety are decreased upon aptamer-driven binding of the target surface moiety to the isolated polynucleotide nanoparticle core.

In certain embodiments, the moiety-coated polynucleotide nanoparticle enabled by this invention increases the activity of the core polynucleotide nanoparticle.

In certain embodiments, the moiety-coated polynucleotide nanoparticle enabled by this invention decreases the activity of the core polynucleotide nanoparticle.

Intramer Aptamers

Nucleic acid based aptamers are single-stranded oligonucleotides composed of ˜20 to 100 nucleotides. DNA, and especially RNA aptamers, exhibit remarkable conformational flexibility and versatility [APT.1] Their unique three-dimensional structure confers specificity for targets ranging from small organic molecules, such as amino acids [APT.2], to large proteins (via small binding regions), to nanometer-sized structures such as liposomes [APT.3]. Additionally, cellular RNA aptamers (aka., intramers) can act as binding sites for amino acids on self-splicing rRNA introns [APT.4], deep binding domains on riboswitches [APT.5, 6], or even as intracellular expression antagonists.

Such aptamers can be selected experimentally through the well-known in-vitro “SELEX” (systematic evolution of ligands by exponential enrichment) combinatorial approach [APT.7, 8] (see Tuerk and Gold, Science 249 (1990), 505-510; Ellington and Szostak, Nature 346 (1990) 818-822). The smallest size of the random region used successfully in a selection is 17 nts (the arginine RNA aptamer) [APT.9], and very short aptamers can be engineered through a truncation of the aptamers obtained from the SELEX procedure: down to 15 nts (thrombin DNA-aptamer) [APT.10] or 13 nts (theophylline RNA-aptamer) [APT.11]. Aptamers bind to targets with high affinity (KD in pico-to-nanomolar range) with exceptional specificity.

However, the long-range binding of isolated aptamers to moieties of low molarity and molecular weight can be limited outside of high-salt environments. The methods of this invention overcome the typical binding limitations of isolated aptamers to free moeities that are at a low molarity and molecular weight within numerous environments, and enable a first-of-its-kind aptamer-driven surface formulation for self-forming polynucleotide nanoparticles.

In preferred embodiments, the individual or group of aptamer(s) and/or intramer(s) within the single-stranded polynucleotide nanoparticle are experimentally selected to recruit a surface moiety by binding specifically to a particular peptide, protein, small molecule, metabolite, organic or inorganic chemical from the results of SELEX, or other Aptamer/moiety binding assays at a lower KD than the isolated aptamer or isolated aptamer group.

In certain embodiments, the isolated or group of randomized aptamer(s) and/or intramer(s) for a given target moiety are transcribed within the single-stranded self-forming polynucleotide nanoparticle used in SELEX, or other aptamer/moiety binding assays.

In other certain embodiments, the isolated or group of randomized aptamer(s) and/or intramer(s) regions for a given target moiety are within the loop region(s) of an RNAi trigger template, and transcribed within the single-stranded self-forming polynucleotide nanoparticle used in SELEX, or other aptamer/moiety binding assays.

In still other certain embodiments, the isolated or group of randomized aptamer(s) and/or intramer(s) regions for a given target moiety are within the loop region(s) of an MV-RNA RNAi trigger or other 3-way junction template, and transcribed within the single-stranded self-forming polynucleotide nanoparticle used in SELEX, or other aptamer/moiety binding assays.

In other embodiments, the isolated or group of randomized aptamer(s) and/or intramer(s) used in SELEX, or other Aptamer/moiety binding assays, are transcribed individually; then later combined with the polynucleotide nanoparticle sequence to create a final surface-forming polynucleotide nanoparticle.

In certain embodiments, the isolated aptamer(s) and/or intramer(s) within the single-stranded polynucleotide nanoparticle are designed to recruit a surface moiety by binding specifically to moieties intracellularly, extracellularly, in-vitro, in-vivo, or any combination thereof.

In certain embodiments, the isolated aptamer(s) and/or intramer(s) within the single-stranded polynucleotide nanoparticle are designed to recruit a surface moiety by binding specifically to moieties that are endogenous, exogenous, or any combination thereof.

In preferred embodiments, aptamer(s) and/or Intramer(s) sequences useful in this invention are experimentally determined using SELEX, or other aptamer binding assay methods, in the manner described above which includes a self-forming polynucleotide nanoparticle with each randomized aptamer-containing transcript.

In other preferred embodiments, aptamer(s) and/or Intramer(s) sequences useful in this invention are chosen from the thousands of known aptamer sequences (i.e., http://aptamer.icmb.utexas.edu) then integrated into the self-forming nanoparticle according to the invention. Examples of such aptamers are shown in Table 1 below.

TABLE 1
Representative Aptamer(s)/Intramer(s)

| SEQ. | | | |
|---|---|---|---|
| ID. NO. | NAME | SEQUENCE (5′→3′) | CITE |
| 1 | Sephadex G- | GUCCGAGUAAUUUACGUUUUGAUAC | Srisawat et al., 2001 |
| | 100 | GGUUGCGGAACUUGC | |
| 2 | HIV-Rev-1 | GGUUUAAUCAGAGUAGAGGAGCUG | Xu & Ellington et al., 1996 |
| | | ACUCCUUUGGUUGGACUA | |
| 3 | Spinach Fluro | GACGCAACUGAAUGAAAUGGUGAAG | Paige et al., 2011 |
| | | GACGGGUCCAGGUGUGGCUGCUUC | |
| | | G | |
| | | GCAGUGCAGCUUGUUGAGUAGAGU | |
| | | GUGAGCUCCGUAACUAGUCGCGUC | |
| 4 | Cyan | GGGAGACGCAACUGAAUGAACCUAG | Paige et al., 2011 |
| | | AGUUAUGCCAGGCUCUGAGCCUGC | |
| | | UUCGGCAGGUGCUAUGAUCGCCAG | |
| | | CGGUAUGCAGUCCGUAACUAGUCG | |
| | | CGUCAC | |
| 5 | HIV-1 Rev-2 | GGUCUGGGCGCAGCGCAAGCUGAC | Bayer et al., 2005 |
| | | GGUACAGACC | |
| 6 | P22N | GGUGCGCUGACAAAGCGCGCC | Bayer et al., 2005 |
| 7 | λ N | GGGCCUGAAGAAGGGCCC | Bayer et al., 2005 |
| 8 | BIV-1 Tat | GGCUCGUGUAGCUCAUUAGCUCCG | Bayer et al., 2005 |
| | | AGCC | |
| 9 | HIV-1 Tat | CCAGAUCUGAGCCUGGGAGCUCUC | Bayer et al., 2005 |
| | | UGG | |
| 10 | HTLV-1 Rex | GCUCAGGUCGAGGTACGCAAGTACC | Bayer et al., 2005 |
| | | UCCCUUGGAGC | |
| 11 | Ricin | CGAAUUCAGGGGACGUAGCAAUGAC | Hesselberth et al., 2000 |
| | | UG | |
| 12 | Mir1-CP | pending | |
| 13 | ZmPep-3 | pending | |
| 14 | Adaptin-CBM | pending | |
| 15 | Lycotoxin | pending | |
| 16 | Du-toxin | pending | |
| 17 | AMP | pending | |

In other specific embodiments, the surface-forming polynucleotide nanoparticle is expressed then recruits peptides onto its surface which evades immune recognition. Many such peptides can be found in given peptidome. However, even a few synthetic peptides have been identified to evade human immune recognition, such as those shown in Table 2 below (P. L. Rodriguez et al., “Minimal ‘self’ peptides that inhibit phagocytic clearance and enhance delivery of nanoparticles,” Science, 339: 971-74, 2013).

TABLE 2
Representative Peptide

| SEQ. | | |
|---|---|---|
| ID. NO. | NAME | SEQUENCE (N → C) |
| 18 | Pep-CD47-self | GNYTCEVTELTREGETIIELK |

Intracellular Surface Formulation

This invention enables the intracellular production of polynucleotide nanoparticles coated with moieties present in a cellular environment. Such moieties would be typically endogenous, transgenic, or even exogenously introduced to the organism used for such intracellular surface formulation of the polynucleotide nanoparticle core expressed within the said cell. A multitude of uses derived from intracellular production and formulation are enabled by this invention.

In some embodiments, the activity, upon oral ingestion of the organism expressing the polynucleotide nanoparticle, is altered due to the surface properties created by this invention. Some such uses are described elsewhere in this application, but include in-planta bio-pesticide production for pests recalcitrant to RNAi, intracellular production of bactericides or fungicides, and even human or animal pharmaceuticals.

In certain embodiments, the surface-forming polynucleotide nanoparticle is expressed and moiety-coated within a host cell selected from a human cell or animal cell or plant cell or yeast cell or insect cell or bacterial cell, or by in-vitro transcription.

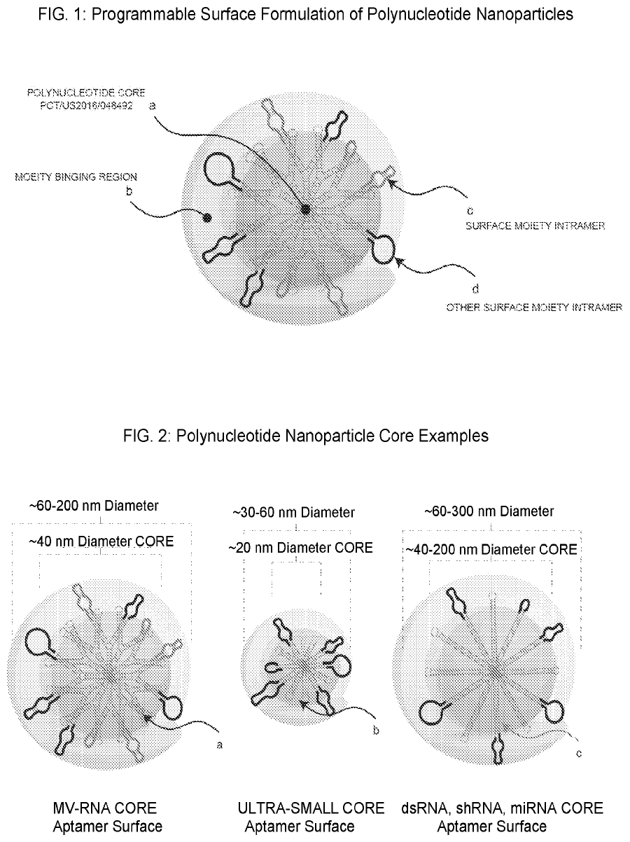

In certain specific embodiments, the surface-forming polynucleotide nanoparticle is produced by intracellular transcription by a promoter (transgenic), virus (transient), or applied topically (exogenic) following in-vitro transcription in a general structure set forth in any one of FIG. 1-2, 4.

Upon oral ingestion, moiety-coated polynucleotide nanoparticles in plant cells are generally protected from stomach from acids and enzymes but are subsequently released into the gut lumen by microbes in humans and animals that digest the plant cell wall. In some insect pests, such as certain hemiptera and lepidoptera, plant cell degrading enzymes are present in the saliva and it is directly the moeity-coated nanoparticle of this invention that provides the nuclease protection and ideal bioavailability characteristics allowing the increase in activity of the polynucleotide nanoparticle core.

In either case, the large mucosal area of the target organism intestine offers an ideal system for oral nanoparticle-based drug delivery. When certain moieties such as receptor-binding peptides, cell-penetrating peptides, endosomal peptides, are used as the polynucleotide nanoparticle coating, organism and cellular specificity can be achieved. The aptamer-driven surface formulation method of this invention provides moiety specificity allowing for programmable features that can provide additional organism selectivity, crossing only the intestinal epithelium of a target organism. A user of this invention is expected to use care when choosing surface moiety candidates in which to design aptamers for the surface-formulation of polynucleotide nanoparticle core. For example, a multitude of unique moieties specific to human or non-human cells, crossing epithelium, blood—brain, or retinal barriers are known in the art and can be applied to this invention, but only a select set would be needed in a particular use.

In some embodiments, the intracellularly produced moiety-coated nanoparticles of this invention have therapeutic purpose in the treatment of cancer, metabolic disorders, neurodegenerative or infectious diseases, but are not limited to these treatments.

In still other embodiments, the intracellularly produced moiety-coated nanoparticles of this invention have therapeutic purpose in the treatment of infectious diseases caused by bacteria or fungus, and the treatment is topical, oral application of cells containing said invention.

In other embodiments, the intracellular production is used for manufacturing of polynucleotide nanoparticles with specifically controlled nanoparticle surface characteristics for medical use.

While plants have been approved by the FDA for the hydroponic production and encapsulation of protein based drugs, in-planta production of polynucleotide nanoparticle with optimized surface characteristics for pharmaceutical use has not been shown in the art. The methods of this invention provide a platform in which future polynucleotide nanoparticle drug production with ideal pharmaceutical properties can be accomplished intracellularly without reliance on viral coat protein encapsulation or dsRNA-binding domain carriers.

Plants offer an ideal alternative to conventional manufacturing systems. Plants are not hosts for human pathogens. The lignin and cellulose packed plant cell wall provides a general natural protection for polynucleotide nanoparticles for human use because humans are incapable of breaking down the glycosidic bonds of the plant cell wall. In humans, gut bacteria digest the plant cell wall and release its contents into the gut lumen.(INPLANTA.13, 14)

Also, plant cells have similar capacity as mammalian cells to produce protein drugs (INPLANTA.15), and could be used to also produce moiety-coated polynucleotide nanoparticle-based drugs by utilizing the methods of this invention. Protein based drug production has been shown in tobacco plants (INPLANTA.16), and carrot cell suspension cultures (INPLANTA.17) and without limits can be used to product the drugs utilizing the methods of this invention.

Similar to mammalian, insect, fungal, and bacterial cells, plant cells can fundamentally facilitate expression, folding, and the self-forming of RNA based structures. Plants stably transformed with transgenes designed using the methods of this invention can be easily propagated from seeds. Agrobacterium tumefaciens is used to deliver such transgenes to the nucleus; whereas a particle delivery system is used to transform plants that are recalcitrant to Agrobacteria-mediated transformation (IN-PLANTA.24).

In some embodiments, chloroplast genomes are used for transformation of the transgenes of this invention.

Chloroplasts have been utilized for stable transformation of numerous heterologous genes since the early 1990s (IN-PLANTA.27-30). The chloroplast genome has a high copy number (>10,000 per cell), enabling transgenes to be expressed at up to 70% of total leaf content (IN-PLANTA.31). Double homologous recombination and transgene integration at target sites eliminate positional effects. In addition, engineering multiple genes into the chloroplast genome is achieved with a single transformation event (IN-PLANTA.32-35) useful in facilitating expression of both the polynucleotide nanoparticle core and even transgenic moieties to be used for surface-binding to the self-forming polynucleotide core transcript. Chloroplasts also sequester the transgene product and complexes within this compartment (IN-PLANTA.21, 36)

Peptide Surface Moieties

A peptidome is a complete set of peptides encoded by a particular genome, or present within a particular cell type or organism, and provides a vast resource of surface moiety candidates relevant to this invention. Example public repositories can be found at NCBI (http://www.ncbi.nlm.nih.gov/peptidome/), or Peptide Atlas (peptideatlas.com), among others. Peptide resources can be searched to locate candidate surface moieties of interest in which to design aptamer/intramer(s) for the surface formulation of the polynucleotide nanoparticles cores according to the methods of this invention.

Peptides have vast applications as surface moieties in this invention, and have a ubiquitous role in gene regulation and immunity in nearly all organisms. Peptides are known to be directly expressed intracellularly in response to stimulus, or massively present in cells as part of the protein degradation process.

In preferred embodiments of this invention, the target surface moiety of the aptamer(s)/intramer(s) is a peptides or protein precursor chosen from the host organisms peptidome.

In certain embodiments of this invention, the target surface moiety of the aptamer(s)/intramer(s) is a peptides or protein precursor chosen from the target organisms peptidome.

In certain embodiments of this invention, the target surface moiety of the aptamer(s)/intramer(s) is a peptides or protein precursor that binds to a secondary peptide or protein that is not targeted by the aptamer(s)/intramer(s) of the polynucleotide nanoparticle.

In other embodiments, the target surface moiety of the aptamer(s)/intramer(s) is a peptide, protein precursor, or protein transiently or transgenically expressed in the host organism.

In still other embodiments, the target surface moiety expression is induced by external stimulus such as pathogen, pest, bio-stress, or chemical means, or other; which leads to the inducible surface coating of the isolated polynucleotide nanoparticle.

In certain embodiments of this invention, the target surface moiety of the aptamer(s)/intramer(s) is a peptide that is cell penetrating, or antimicrobial, or antifungal, or endosomally disruptive, or endosomally escaping, translocating, cell signaling, receptor-binding, or toxic to a target organism, or any mix thereof.

To computationally calculate the peptide residue properties such as hydropathy, MW, Iso-electric point, and Net charge at a given pH, an online tool (http://pepcalc.com) provides both data and graphics profiling a given peptide sequence. For this invention, it is important understand these basic peptide characteristics when selecting a surface moiety candidate in which to develop a specific binding aptamer/intramer.

In certain embodiments of this invention, the target surface moiety of the aptamer(s)/intramer(s) is a peptide that is hydrophobic, or hydrophilic, or cationic, or annionic, or degradation resistance, or any mix thereof.

In preferred embodiments of this invention, the target surface moiety of the aptamer(s)/intramer(s) is a peptide that has a linear residue length of 6 or more.

In still other preferred embodiments of this invention, the target surface moiety of the aptamer(s)/intramer(s) are peptides that has a net charge of −20, −14, −9, −6, −2, neutral, +1, +4, +6, +14, or higher at pH 7.

In certain embodiments of this invention, the target surface moiety of the aptamer(s)/intramer(s) is a peptide that is due to a immunological response of the host.

In still other certain embodiments of this invention, the target surface moiety of the aptamer(s)/intramer(s) is a peptide that is not a endogenous peptide of the host.

In still other certain embodiments of this invention, the target surface moiety of the aptamer(s)/intramer(s) is a peptide that is secreted from a cell including, but not limited to, those shown in Table 3 below.

TABLE 3
Representative Peptide

| SEQ. | | |
|---|---|---|
| ID. | | |
| NO. | NAME | SEQUENCE (N → C) |
| 19 | pep-HIV Rev | TRQARRNRRRRWRERQR |
| 20 | pep-P22 N | NAKTRRHERRRKLAIER |
| 21 | pep-λ N | MDAQTRRRERRAEKQAQWKAAN |
| 22 | pep-BIV Tat | SGPRPRGTRGKGRRIRR |
| 23 | pep-HIV-1 Tat | SYGRKKRRQRRRPPQ |
| 24 | pep-HTLV-1 Rex | MPKTRRRPRRSQRKRP |
| 25 | pep-Mir1-CP | SYPVKDTYHPGTGTATARAAAMDVIKMVLA |
| 26 | pep-ZmPep 3 | TRTPPWPPCPPEEGSGGNGGSHN |
| 27 | Citrus Mir1-CP | SVSLEDTYRPGKGTS |
| 28 | Apple Mir1-CP | SVSLEDTYRPGKGTS |

Nuclease Degradation Resistance

In certain aspects of the invention, a general improvement of bioavailability is realized due to increased stability of the moiety-coated polynucleotide nanoparticles in blood or hemolymph, saliva, or gut of the target organism.

In preferred embodiments, an increase in the nuclease degradation resistance of isolated polynucleotide nanoparticle core is evident upon the aptamer-driven binding of four or more target surface moieties.

In other aspects of the invention, the nuclease stability can be tuned by increasing the number of aptamer/intramer(s) within the polynucleotide nanoparticle core sequence to control both the number and type of bound surface moieties. For example, one may increase the number of surface moieties for certain Lepidoptera insects where significant enzymatic activity is present. In contrast, a lower titration of surface moieties may be desired when using this invention to transfect cell cultures. In all cases, one will want to impart stability upon the polynucleotide nanoparticle core by using the methods of this invention, but will want avoid over-stabilizing the isolated polynucleotide core with an over-abundance of bound surface moieties which can impede the activity of the isolated polynucleotide nanoparticle core.

In other preferred embodiments, appropriate nuclease degradation resistance of isolated polynucleotide nanoparticle core is provided upon the aptamer-driven binding of 4, 6, 12, 24, 64, or 128 target surface moieties, and all number in between.

Cell Penetrating Peptides (CPPs)

CPPs represent a large peptide family with different biochemical characteristics (Laufer et al., 2012; Millet, 2012; El-Sayed and Harashima, 2013).

In specific embodiments of this invention, the target surface moiety of the aptamer(s)/intramer(s) is a cell-penetrating peptide between 6-30 amino acids in length.

In general, CPP's are cationic, partially hydrophobic, or partially amphiphilic, or periodic peptides that can translocate across cell membranes. There are many different CPP's in the art with various characteristics that have been shown effective in animal cell models treated with cationic CPPs (Ziegler et al., 2005; Tünnemann et al., 2006; Rinne et al., 2007; Kosuge et al., 2008; Tanaka et al., 2012; Liu et al., 2013), and insect cells (Cermenati et al., 2011; Chen et al., 2012; Pan et al., 2014; Zhou et al., 2015). The methods of this invention further enhance the use of CPP's across a broad pH range and cellular uptake modalities with aptamer-driven binding combined with electrostatic attraction of these cationic peptides.

CPPs are fairly expensive to synthesize and cost is an issue when it comes to the scale required for animal or human studies.

This methods of this invention allow for the intracellular production of either endogenous or transgenic CPP and automatic formulation onto the surface of a self-forming polynucleotide nanoparticle useful for medical uses.

In other specific embodiments of this invention, the target surface moiety of the aptamer(s)/intramer(s) is a cell-penetrating peptide that is expressed within a cell along with the polynucleotide nanoparticle core.

Secondly, peptide CPP's do not have any oral bioavailability and to date have been delivered clinically either through topical, or intravenous applications.

In aspects of this invention, the bioavailability of CPP's are increased when used as a surface moiety.

Thirdly, issue relates to the non-specific uptake of the cationic and hydrophobic CPPs is overcome by the methods of this invention since they can be programmatically assigned with other receptor-targeting peptides on the surface of the polynucleotide nanoparticle core; adding a specificity function to the membrane translocation features of CPP's.

In other specific embodiments, the cellular or membrane penetration rate of the moeity-coated polynucleotide nanoparticles of this invention are increased due to the controlled surface composition containing specific orientations of cell penetrating peptides.

In certain aspects of this invention, cell penetrating peptides are bound to the polynucleotide nanoparticle core by aptamer/intramer(s).

In certain aspects of this invention, the presentation of the hydrophobic and/or cationic portion of a bound CPP moiety is clustered using a plurality of aptamer-driven CPP binding as to change a portion of the nanoparticle surface to hydrophobic and/or cationic; thus offering a programmable cellular membrane disruption feature using the methods of this invention.

In preferred embodiments of this invention, the target surface moiety of the aptamer(s)/intramer(s) is a cell-penetrating peptide effective in specifically penetrating the target cell, such as those shown below in Table 4.

TABLE 4
Representative Peptide

| SEQ. | | | |
|---|---|---|---|
| ID. | | | |
| NO. | NAME | SEQUENCE (N → C) | REF. |
| 29 | Penetratin | RQIKWFQNRRMKWKK | |
| 30 | TAT | YGRKKRRQRRR | |
| 31 | SynB1 | RGGRLSYSRRRFSTSTGR | |
| 32 | SynB3 | RRLSYSRRRF | |
| 33 | PTD-4 | PIRRRKKLRRLK | |
| 34 | PTD-5 | RRQRRTSKLMKR | |
| 35 | FHV Coat- | RRRRNRTRRNRRRVR | |
| | (35-49) | | |
| 36 | BMV Gag- | KMTRAQRRAAARRNRWTAR | |
| | (7-25) | | |
| 37 | HTLV-II Rex- | TRRQRTRRARRNR | |
| | (4-16) | | |
| 38 | D-Tat | GRKKRRQRRRPPQ | |
| 39 | R9-Tat | GRRRRRRRRRPPQ | |
| 40 | Transportan | GWTLNSAGYLLGKINLKALAALAKKIL | |
| | | chimera | |
| 41 | MAP | KLALKLALKLALALKLA | |
| 42 | MAIZE SAP | VRLPPP | |
| 43 | FBP | GALFLGWLGAAGSTMGAWSQPKKKRKV | |
| 44 | MPG | ac-GALFLGFLGAAGSTMGAWSQPKKK | |
| | | RKV-cya | |
| 45 | MPG(ΔNLS) | ac- | |
| | | GALFLGFLGAAGSTMGAWSQPKSKRKV- | |
| | | cya | |
| 46 | Pep-1 | ac-KETWWETWWTEWSQPKKKRKV-cya | |
| 47 | Pep-2 | ac-KETWFETWFTEWSQPKKKRKV-cya | |

Endosomal Escape

In addition of CPP's, this invention, according to some embodiments, may further increase the rate of endosomal escape of a moiety-coated polynucleotide nanoparticle with the use of endosomolytic, or clathrin-pit binding, or a series of peptides of differing isoelectric points as to create a surface that responds to the pH gradient of early and late endosomes, or mix thereof.

In certain embodiments of this invention, the aptamer-targeted surface moiety of the aptamer(s)/intramer(s) is a endosomolytic peptide.

In certain embodiments of this invention, the aptamer-targeted surface moiety of the aptamer(s)/intramer(s) is a Clathrin-pit endosomal receptor-binding peptide.

In specific embodiments of this invention, the aptamer-targeted surface moieties of the aptamer(s)/intramer(s) are peptides of differing isoelectric points resulting in a ampholytic polynucleotide nanoparticle surface upon formulation.

A multitude of uptake routes are offered by utilizing the methods of this invention. In general, surface compositions can greatly influence the uptake rate and route of uptake via endocytosis, micropinocytosis or macropinocytosis. More so, nanoparticle diameter in combination with surface characteristics significantly contribute to uptake efficiencies during vesicle formation. Nanoparticle diameters below 60 nm allow for efficient uptake without exhausting limited membrane receptors critical for cellular health. The compositions and methods of this invention provide ideal nanoparticle diameters with programmable surface characteristics adaptable for particular cellular barriers. The tunable surface features created by using this invention allow for tuning endosomal transport during the maturation of vesicles into late endosomes during endocytosis, at which point the pH becomes highly acidic (El-Sayed and Harashima, 2013).

In certain embodiments of this invention, intracellularly moiety-coated polynucleotide nanoparticles are efficiently engulfed into vesicles with a diameter below 200 nm when endocytosis or micropinocytosis is preferred.

In other embodiments of this invention, extracellularly moiety-coated polynucleotide nanoparticles are engulfed into vesicles with a diameter greater than 200 nm when macropinocytosis is preferred.

In still other embodiments, the endosomal escape rate of the moiety coated polynucleotide nanoparticles created using the methods of this invention are increased over that of isolated polynucleotide nanoparticle cores, such as those shown in Table 5 below.

TABLE 5
Representative Peptide

| SEQ. | | | |
|---|---|---|---|
| ID. NO. | NAME | SEQUENCE (N → C) | REF. |
| 48 | ENDOSOMALYTIC_ | RGWEVLKYWWNLLQY | |
| | TRUN_HGP | | |
| 49 | Clathrin-CBM | ETLLDLDFDP | |
| 50 | Clathrin-4T1 | ETLLDLDFLERPHRD | |
| 51 | Clathrin-AP2 | GDLLNLDLGPPV | |
| 52 | LMW Proatamine | TDSP5 (VSRRRRRRGGRRRR) | |

Multimodality

In certain embodiments, the isolated polynucleotide nanoparticle core targets genes of insects, or virus, or fungus, or animals, or humans, or host plant (FIG. 8a), other plants, or any combination thereof; and the surface moiety targets insects, or virus, or bacteria, or fungus, or animals, or humans, or host plant, or any combination thereof by using a general structure set forth in any one of (FIG. 8b).

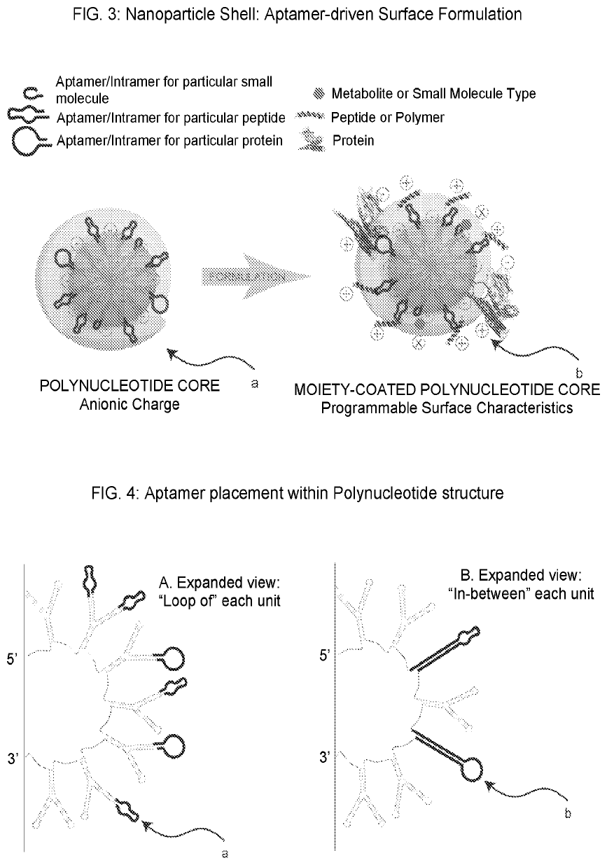

In certain embodiments, additional mode(s) of action are added upon aptamer-driven binding of the target surface moiety or moieties. (FIG. 3b, 9, 10, 11).

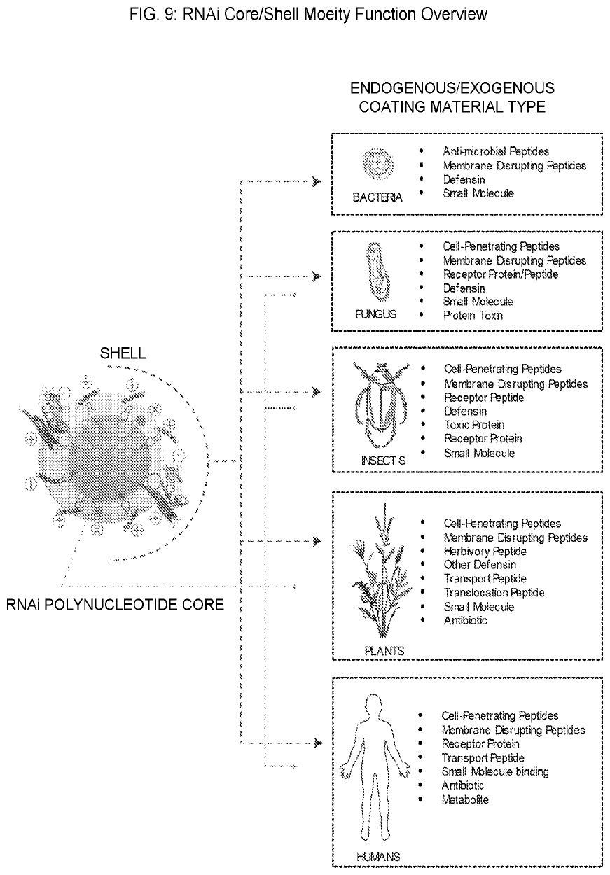

In specific embodiments of this invention, the isolated polynucleotide nanoparticle is gene modulating by RNAi, but the moiety-coated surface adds an additional function as a anti-microbial, anti-fungal, pesticide, or both (FIG. 9, 11b-c).

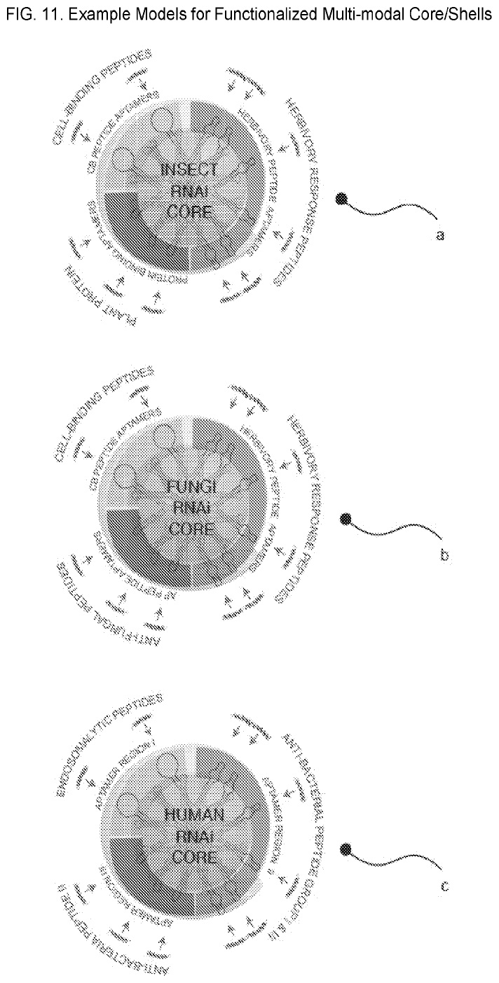

In still other specific embodiments, the isolated polynucleotide nanoparticle is a single polynucleotide nanoparticle coated with anti-microbial peptides, antifungal peptides, toxic peptides, or toxic proteins, or combination thereof. (FIGS. 8-11).

In specific embodiments, the isolated polynucleotide nanoparticle changes surface charge, nuclease resistance, protease resistance, mode of action, and molecular weight upon the aptamer-driven binding of the target surface moiety or moieties (FIG. 3b, 10, 11).

In other specific embodiments, the surface charge of isolated polynucleotide nanoparticle becomes less anionic, neutral, cationic, or mix thereof upon the aptamer-driven binding of the target surface moiety or moieties (FIG. 3b, 10, 11).

Receptor Peptides

In other specific embodiments, the isolated polynucleotide nanoparticle targets genes in organisms other than those of the host. Organism specificity can be determined by complementarity of the polynucleotides to the target genes and cellular uptake signals such as aptamers, ligands, linkage nucleotides, loops, long dsRNA, ssRNA ends, function of bound surface moieties, or a combination thereof (FIG. 9).

In certain embodiments, the aptamer-targeted surface moieties of the aptamer(s)/intramer(s) are peptides to cell or organism specific receptors.

In preferred embodiments, the aptamer-targeted surface moieties of the aptamer(s)/intramer(s) are peptides to cell or organism specific receptors that 6-12 amino acids in length.

In other preferred embodiments, the aptamer-targeted surface moieties of the aptamer(s)/intramer(s) are peptides to cell or organism specific receptors that 6-30 amino acids in length.

In still other embodiments, the aptamer-targeted surface moieties of the aptamer(s)/intramer(s) are peptides to cell or organism specific receptors that are longer than 30 amino acids in length, such as those shown in Table 6 below.

TABLE 6
Representative Peptide

| SEQ. | | | |
|---|---|---|---|
| ID. NO. | NAME | SEQUENCE (N → C) | REF. |
| 53 | Binding- Cry_boost | P1Z: CHLPRLPQC; P2S: | 20106 (2016) |
| | | CLMSSQAAC | doi: 10.1038/srep20106 |
| 54 | Aphid GBP3.1 | TCSKKYPRSPCM | Liu, et al., 2010 |
| 55 | S. Frugiperda.CAD | FLDRLSATEDGLHAGRVTFSIAGNDE | Rahman, et al., 2012 |
| | | AAEYFNVLNDGDNSAMLTLKQALPAG | |
| | | VQQFELVIRATDGGTEPGPRSTDCSV | |
| | | TVVFVMTQGDPVFDDNAASVRFVEK | |
| | | EAGMSEKFQLPQADDPKNYRCMDDC | |
| | | HTIYYSIVDGNDGDHFAVEPETNVIYL | |
| | | LKPLDRSQQEQYRVVVAASNTPGGT | |
| | | STLSSSLLTVTIGVREANPRPIFESEFY | |
| | | TAGVLHTDSIHKELVYLAAKHSEGLPI | |
| | | VYSIDQETMKIDESLQTVVEDAFDINS | |
| | | ATGVISLNFQPTSVMHGSFDFEVVAS | |
| | | DTRGASDRAKVSIYMISTRVRV | |
| 56 | M. sexta.CAD | HLERISATDPDGLHAGVVTFQVVGDE | Rahman, et al., 2012 |
| | | ESQRYFQVVNDGANLGSLRLLQAVP | |
| | | EEIREFRITIRATDQGTDPGPLSTDMT | |
| | | FRVVFVPTQEPRFASSEHAVAFIEKSA | |
| | | GMEESHQLPLAQDIKNHLCEDDCHSI | |
| | | YYRIIDGNSEGHFGLDPVRNRLFLKKE | |
| | | LIREQSASHTLQVAASNSPDFGIPLPA | |
| | | SILTVTVTVREADPRPVFMRELYTAGI | |
| | | STADSIGRELLRLHATQSEGAAITYAID | |
| | | YDTMVVDSLEAVRQSAFVLNAQTGVL | |
| | | TLNIQPTATMHGLFKFEVTATDTAGA | |
| | | QDRTDVTVYVVSSQNRLE | |
| 57 | D. virgifera.CAD | MAVLMTKDLQCSENLNKDGEVGEGII | Park, et al., 2009 |
| | | GEDIDDGDNAKIDFSVLSIVDKETKNDI | WO 2009023636 A1 |
| | | QESFNISKIDSDYVLNDTLKKVHLIAFE | |
| | | DLKGKYGTYEVTLHMHDEGDPMQTT | |
| | | DPDPTLTLTIEKWNYQTPSIIFPENDQ | |
| | | TYIVLSDQQPGQPLALFNNTGTSNTL | |
| | | PDFSATDGETKDYSKWDVKFSYTQT | |
| | | NYEDDKIFVIDHIQPCVSQLQVSKHFN | |
| | | SDLVRSKKYKLTITASVKDGAEQEGE | |
| | | AGYSTSANISIVFLNNDAQPIFQNSDW | |
| | | SVSFVEFNTTQPAKPLEEQAEYENTK | |
| | | GGLPIYYHFYSENQTLSKYFEVDETS | |
| | | GDLSVIGNLTYDYDQDISFHIVASNDS | |
| | | QVRMLDPRSSLNVTVNFLPRNRRAP | |
| | | QWHHHHHH (Cry3Bb, Cry3Aa binding | |
| | | site BOLD) | |
| 58 | SV40_nuclear_sig | PKKRKV | |
| 59 | MPG-nucLoc | GLAFLGFLGAAGSTMGAWSQPKKKR | |
| | | KV | |
| 60 | apoptotic_pep | KLAKLAK | Kwon, et al., 2008 |
| 61 | Bp100 | KKLFKKILKYL | Badosa, et al., 2007 |
| 62 | Tat | RKKRRQRRRRKKRRQRRR | Chugh, et al., 2007 |

Aptamer-Driven Moiety Clusters

In preferred embodiments, the moiety-coated polynucleotide nanoparticle enabled by this invention increases the activity of the isolated surface moiety by grouping moieties onto surface regions of the polynucleotide nanoparticle core.

Pathogenic bacterial toxins that target cell membranes possess a similar functional construction, referred to as a ABE model. It has been observed in well characterized toxins, such as cholera and shigella, that a “B” domain functions in binding to cell surface receptors, while an “A,” or activity, domain exerts the toxin's specific biological activity (ABE.68, ABE.52). A and B domains may be synthesized together or separately. An additional separate region of hydrophobicity is called “E” (entry domain), and plays a role in facilitating insertion of the toxin after receptor binding (ABE.68).

The method of this invention allows for the mimicking of the ABE model indicative of a toxin structure. Such ABE model may be analogous to the domains of CrylAc, and mimicking of this model using the methods of this invention provides for a useful tool in creating flexible pesticides modalities without the tedious search for additional bacterial toxins.

With this understanding, one can utilize this invention to simulate surface polarity by grouping aptamers within the polynucleotide transcript so that hydrophobic presentation is localized within a particular region of the final moiety-coated polynucleotide nanoparticle (FIG. 10).

To determine hydrophobicity of peptide candidates to be potentially used in this manner, online tools such as (http:pepcalc.com) can provide preliminary calculations of hydrophobicity.

In certain embodiments, the moiety-coated polynucleotide nanoparticle enabled by this invention mimics protein surface characteristics by binding specific moieties onto particular regions of the polynucleotide nanoparticle core, for example, those shown in Table 7 below.

TABLE 7
Representative ABE Peptide Set

| SEQ. | | | |
|---|---|---|---|
| ID. NO. | NAME | SEQUENCE (N → C) | REF. |
| 63 | Active- | HHHHHHDDDKIWLTALKFLGKHAAKHLAKQQLSP | U.S. GOV |
| | Lycotoxin | W | |
| 64 | Binding- | P1Z: CHLPRLPQC; P2S: CLMSSQAAC | Scientific Reports 6, |
| | Cry_boost | | Article number: 20106 |
| | | | (2016) |
| | | | doi: 10.1038/srep20106 |
| 65 | Entry CPP | KHKHKHKHKHKHKHKHKHKKLFKKILKYL | |

In certain embodiments, the moiety-coated polynucleotide nanoparticle enabled by this invention results in a polar nanoparticle by binding cationic moieties onto the opposite end of either bound anionic moieties, or the isolated anionic polynucleotide nanoparticle core itself.

Antimicrobial Peptides

The methods of this invention enable programmable moiety-coated polynucleotide nanoparticles with novel secondary antimicrobial modes of action over that of isolated antimicrobial peptides.

Antimicrobial peptides (AMPs) from different organisms have been characterized to date. AMPs are small molecular weight peptides that are typically less than 55 amino acid residues in length, and have broad spectrum antimicrobial activity against Gram-positive and Gram-negative bacteria, viruses, and fungi (AMP.1). These endogenous polypeptides are produced by multicellular organisms in order to protect a host from pathogenic microbes—playing an essential role in innate immune responses (AMP.1,2).

In general, AMPs fold into membrane environments, presenting one positively charged side (mainly due to lysine and arginine residues), and other side with a considerable proportion of hydrophobic residues (AMP.1, AMP.3, 4). Their cationic properties lead to selective interaction with the negatively charged surfaces of microbial membranes, resulting in the accumulation of AMPs on the membrane surface. The hydrophobic portions appear responsible for the interaction with hydrophobic components of the membrane. These interactions and structural associations may result from the formation of peptide-lipid specific interactions, and lead to translocation across the membrane, or the most common mechanism, a membranolytic effect (AMP. 2,3).

In preferred embodiments, the aptamer-targeted surface moieties of the aptamer(s)/intramer(s) are antimicrobial peptides.

In other preferred embodiments, the aptamer-targeted surface moieties of the aptamer(s)/intramer(s) is a single antimicrobial peptides bound in plurality to the polynucleotide nanoparticle core in a clusters or groups.

In medicine, AMPs are considered a potential class of antibiotic because of their broad-spectrum activities and different mechanisms of action compared to conventional antibiotics. Although AMPs possess considerable benefits as new generation antibiotics, their clinical and commercial development still have some limitations, such as poor bioavailability, potential toxicity, susceptibility to proteases, and high cost of production.

The compositions and methods of this invention overcome obstacles for using AM P's efficiently in medicine. Importantly, the general bioavailability of these low molecular weight polypeptides is increase by utilizing them as the surface moiety on the larger polynucleotide nanoparticle core.

However, additional benefits of this invention are realized by the aptamer-driven grouping of particular classes of AMP's onto the surface of a polynucleotide nanoparticle core to supplement the AMP's mechanism of action in translocation and/or membrane disruption.

In preferred embodiments, the aptamer-targeted surface moieties of the aptamer(s)/intramer(s) are exclusively selected antimicrobial peptides to a particular microbial target spectrum.

In preferred embodiments, the aptamer-targeted surface moieties of the aptamer(s)/intramer(s) are antimicrobial peptides which become bound to the polynucleotide nanoparticle core surface extracellularly (i.e. topically).

In aspects of this invention, polynucleotide nanoparticle cores with aptamer-driven binding to AMP's present in an extracellular or topical setting can be introduced in order to facilitate the binding of the target AMP moieties onto the nanoparticle surface, hence enabling the benefit of the final moiety coated nanoparticle. In non-limited examples, such methods would be useful in the treatment or prevention of infections of skin, eyes, bladder, blood by supplementing the mechanism of action of the AMP's present in each setting.

Plant diseases caused by viruses, bacteria and fungi affect crops, and have an effect on significant losses or decrease the quality and safety of agricultural products (AMP.5). In order to reduce yield loss, development of novel crop-protection strategies is urgently needed. Plants have long been known to exhibit mechanisms that enabled them to detect and defend against microbial attack. In response to microbial attack, plants have activated a complex series of responses that lead to the local and systemic induction of a broad-spectrum of antimicrobial defenses (AMP.6).

Cathelicidins, defensins and thionins are the three major groups of epidermal AMPs in human and plants. Plant AMPs are structurally and functionally diverse and can be directed against other organisms, like herbivorous insects. Several antimicrobial peptides have been expressed in transgenic plants to confer disease protection. Endogenous antimicrobial peptides are promising compounds that can be exploited for disease control in plants, and comply with the strict regulations on the safety of disease control. AMPs from various sources have been demonstrated to confer resistance against fungal and bacterial pathogens in an array of genetically engineered plant species, including Arabidopsis (AMP.7), tobacco (AMP.8,9,10), Chinese cabbage (AMP.11), rice (AMP.12,13), tomato (AMP.14), cotton (AMP.15), potato (AMP.16), pear (AMP.17), banana (AMP. 8) and hybrid poplar (AMP.18).

In aspects of this invention, endogenous AMP's are re-invigoration with a new bioavailability profile and are presented in novel compositions to the pests that have grown accustom to isolated AMP's through evolution or commercial use.

In preferred embodiments, the aptamer-targeted surface moieties of the aptamer(s)/intramer(s) are antimicrobial peptides which become bound to the polynucleotide nanoparticle core surface intracellularly.

In preferred embodiments, the aptamer-targeted surface moieties of the aptamer(s)/intramer(s) are antimicrobial peptides which become bound to the polynucleotide nanoparticle core surface extracellularly (i.e. topically).

A peptides theoretical antimicrobial potential can be predicted on the basis of sequence with special AMPA software (http://tcoffee.crg.cat/apps/ampa/do) [AMP.41]. For AMPA analysis, minimum recommended parameters are; threshold value: 0.225, window size: 7 amino acids, misclassification: <5%.

Additional AMP's are available online in publication or databases such as APD3 (http://aps.unmc.edu/AP/). This database currently focuses on natural antimicrobial peptides (AMPs) with defined sequence and activity. It includes a total of 2619 AMPs with 261 bacteriocins from bacteria, 4 AMPs from archaea, 7 from protists, 13 from fungi, 321 from plants and 1972 animal host defense peptides. The APD3 contains 2169 antibacterial, 172 antiviral, 105 anti-HIV, 959 antifungal, 80 antiparasitic and 185 anticancer peptides. Newly annotated are AMPs with antibiofilm, antimalarial, anti-protist, insecticidal, spermicidal, chemotactic, wound healing, antioxidant and protease inhibiting properties. Nearly all AMP's in this database are useful for the aptamer-driven surface formulation methods of this invention, for example, those shown in Tables 8 and 9 below.

TABLE 8
Representative Anti-bacterial Peptides

| SEQ. | | | |
|---|---|---|---|
| ID. | | | |
| NO. | NAME | SEQUENCE (N → C) | REF. |
| 66 | HNP1 | ACYCRIPACIAGERRYGTCIYQGRLWAFCC | Valore, et al. 1996 |
| 67 | hBD1 | DHYNCVSSGGQCLYSACPIFTKIQGTCYRGKAKCCK | Niyonsaba, et al. |
| | | | 2005 |
| 68 | Pilosulin-1 | GLGSVFGRLARILGRVIPKV | |
| 69 | Ostricacin-2 | APGNKAECEREKGYCGFLKCSFPFVVSGKCSRFFFC | |
| | | CKNIW | |

TABLE 9
Representative Anti-fungal Peptide

| SEQ. | | | |
|---|---|---|---|
| ID. NO. | NAME | SEQUENCE (N → C) | REF. |
| 70 | PDC1 | ARVCMGKSQHHSFPCISDRLCSNECVKEDGGWTAG | Kant, et al. 2009 |
| | | YCHLRYCRCQKAC | |
| 71 | rust_NaD1 | RECKTESNTFPGICITKPPCRKACISEKFTDHCSKILR | Dracatos, et al. 2013 |
| | | RCLCTKPC | |
| 72 | HsAFP1 | DGVKLCDVPSGTWSGHCGSSSKCSQQCKDREHFA | Osborn, et al. 1995 |
| | | YGGACHYQFPSVKCFCKRQC | |
| 73 | Lactoferricin | FKCRRWQWRMKKLGAPSITCVRRAF | Bellamy, et al. 1992 |
| | B | | |
| 74 | MBP1 | RSGRGECRRQCLRRHEGQPWETQECMRRCRRRG | Duvick, et al. 1991 |
| 75 | RsAFP2 | KLCQRPSGTWSGVCGNNNACKNQCIRLEKARHGSC | Terras, et al. 1991 |

Defensins

Defensins are approx. 2-6 kDa, cationic, microbicidal peptides active against many Gram-negative and Gram-positive bacteria, fungi, and enveloped viruses. Defensins are produced constitutively and/or in response to microbial products, proinflammatory cytokines, or herbivory responses in plants. The mechanism(s) by which microorganisms are killed and/or inactivated by defensins is not fully understood.

Currently, it is generally believed that function is due to disruption of the microbial membrane. Similar to ABE protein toxins, defensins exhibit generally a polar topology, with separated charged and hydrophobic regions. This common theme in nature likely contributes to insertion into the phospholipid membranes so that their hydrophobic regions are buried within the lipid membrane interior and their charged (mostly cationic) regions interact with anionic phospholipid head groups and water.

Additionally, some defensins can aggregate to form ‘channel-like’ pores; others might bind to and cover the microbial membrane in a ‘carpet-like’ manner. Either way, the outcome is a disruption of membrane integrity.

Biopesticides

RNAi has demonstrated a commercial potential to control insect pests. However, the efficiency of RNAi can vary greatly between the different insect orders. In many RNAi recalcitrant insect species, the gene knockdown is low or ineffective at low concentrations (Huvenne and Smagghe, 2010; Li et al., 2013).

Efficient uptake of RNA by the epithelial cells of the insect midgut is fundamental to the effectiveness of in-planta protection using RNAi. Aside from coleoptera (Baum et al., 2007; Zhu et al., 2011; Bolognesi et al., 2012; Rangasamy and Siegfried, 2012), little progress has been in made in overcoming the two greatest barriers; oral bioavailability, and cellular uptake.

The aptamer-driven surface formulation methods of this invention utilize intracellular and in-vitro production to solve; oral bioavailability, nuclease stability, multi-modal activity, and cellular uptake challenges of insects currently recalcitrant to plant incorporated protectants (PIP).

In methods of this invention, a plants own peptidome, which contains hebivory response peptides, can be used to overcome nuclease degradation, provide multimodal activity, optimize the surface charge to increase penetration into the peritrophic matrix in both a stable or inducible manner.

In preferred embodiments, the aptamer-targeted surface moieties of the aptamer(s)/intramer(s) are peptides derived from the peptidome of the host.

In preferred embodiments, the aptamer-targeted surface moieties of the aptamer(s)/intramer(s) are bound intracellularily, or extracellularly, in-vivo, or in-vitro.

In specific embodiments, the isolated polynucleotide nanoparticle is gene modulating by RNAi, but coating surface moiety is a non-toxic peptide, a toxic peptide, or combination of.

In specific embodiments, the isolated polynucleotide nanoparticle is gene modulating by RNAi, but coating surface moiety contains a Damage Associated Microbial Pattern, a Microbe Associated Molecular Pattern, or combination of.

In specific embodiments, the isolated polynucleotide nanoparticle is gene modulating by RNAi, but coating surface moiety is toxic protein, chemical, or combination of.

In specific other embodiments, the isolated polynucleotide nanoparticle is gene modulating by RNAi, but coating surface moiety is toxic protein, chemical, or combination of.

A source of MAMPs is insect venom. Such compounds are toxic to other insects, and could provide biological control of agricultural pests. In particular, spider venom is a potential source of novel insect-specific peptide toxins.

One example is the small amphipathic \hat{l}±-helical peptide lycotoxin-1 (Lyt-1 or LCTX) from the wolf spider (Lycosa carolinensis). The positive charge of the hydrophilic side interacts with negatively charged prokaryotic membranes and the hydrophobic side associates with the membrane lipid bilayer to permeabilize it. The exoskeleton surface of an insect is highly hydrophobic, and an amphipathic compounds offers a strong method to permeabilize it.

The methods of this invention allow for increase bioavailability, programmable hydrophobic presentation, leading to the use of toxins at a lower molarity.

In certain embodiments, partial surface formation and ultimate bioavailability is induced intracellularly upon the binding of DAMP or MAMMP containing peptides triggered by an Herbivory stimulus, for example, those shown in Table 10 below.

TABLE 10
Representative Peptide

| SEQ. | | | |
|---|---|---|---|
| ID. NO. | NAME | SEQUENCE (N → C) | REF. |
| 76 | Dupont_NH2 | RSDSRGFVLSLKKNGAQH | DuPont Patent |
| 77 | Dupont_NH2-Mod | KHKHKHKHRSDSRGFVLSLKKNGAQH | DuPont Patent |
| 78 | Lycotoxin | H2N- | U.S. Gov. Patent |
| | | HHHHHHDDDKIWLTALKFLGKHAAKHLAKQQLSPW- | |
| | | COOH | |
| 79 | Florida_NPF | ARGPQLRLRF | U of Florida |
| | | | Patent |

Pathogens of the invention include, but are not limited to, viruses or viroids, bacteria, insects, nematodes, fungi, and the like. Viruses include any plant, animal, or human virus, for example, tobacco or cucumber mosaic virus, HIV, HBV, HSV, HPV, ringspot virus, necrosis virus, maize dwarf mosaic virus, etc. Specific fungal and viral pathogens for the major crops include: Soybeans: Phytophthora megasperma f.sp. glycinea, Macrophomina phaseolina, Rhizoctonia solani, Selerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotrichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines, Fusarium solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Altemaria alternata; Alfalfa: Clavibacter Michigan's subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium spp., Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Tilletia indica, Pythium gramicola, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum p.v. carotovora, Cephalosporium acremonium, Phytophthora cryptogea, Albugo tragopogonis; Corn: Fusarium moniliforme var. subglutinans, Erwinia stewartii, Fusarium moniliforme, Gibberella zeae (Fusarium graminearum), Stenocarpella maydis (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudomonas avenae, Erwinia chrysanthemi p.v. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Periconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.

Nematodes include parasitic nematodes such as root-knot, cyst, and lesion nematodes, including Heterodera and Globodera spp.; particularly Globodera rostochiensis and Globodera (potato cyst nematodes); Heterodera glycines (soybean cyst nematode); Heterodera schachtii (beet cyst nematode); and Heterodera avenae (cereal cyst nematode). Additional nematodes include: Heterodera cajani; Heterodera trifolii; Heterodera oryzae; Globodera tabacum; Meloidogyne incognita; Meloidogynejavonica; Meloidogyne hapla; Meloidogyne arenaria; Meloidogyne naasi; Meloidogyne exigua; Xiphinema index; Xiphinema italiae; Xiphinema americanum; Xiphinema diversicaudatum; Pratylenchus penetrans; Pratylenchus brachyurus; Pratylenchus zeae; Pratylenchus coffeae; Pratylenchus thomei; Pratylenchus scribneri; Pratylenchus vulnus; Pratylenchus curvitatus; Radopholus similis; Radopholus citrophilus; Ditylenchus dipsaci; Helicotylenchus multicintus; Rotylenchulus reniformis; Belonolaimus spp.; Paratrichodorus anemones; Trichodorus spp.; Primitivus spp.; Anguina tritici; Bider avenae, Subanguina radicicola; Tylenchorhynchus spp.; Haplolaimus seinhorsti; Tylenchulus semipenetrans; Hemicycliophora arenaria; Belonolaimus langicaudatus; Paratrichodorus xiphinema; Paratrichodorus christiei; Rhadinaphelenchus cocophilus; Paratrichodorus minor; Hoplolaimus galeatus; Hoplolaimus columbus; Criconemella spp.; Paratylenchus spp.; Nacoabbus aberrans; Aphelenchoides besseyi; Ditylenchus angustus; Hirchmaniella spp.; Scutellonema spp.; Hemicriconemoides kanayaensis; Tylenchorynchus claytoni; and Cacopaurus pestis.

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, sugarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blotch leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; eleodes, Conoderus, and aeolus spp., wireworms; Oulema melanopus, cereal leaf beetle; Chaetocnema pulicaria, corn flea beetle; Sphenophorus maidis, maize bilibug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worn; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia spp., Root maggots.

Methods of Regulating Gene Expression

A target gene may be a known gene target, or, alternatively, a target gene may be not known, i.e., a random sequence may be used. In certain embodiments, target mRNA levels of one or more, preferably two or more, target mRNAs are reduced at least 10%, at least 20%, at least 30%, at least 40%, at least 50%, at least 60%, at least 70%, at least 75%, at least 80%, at least 90%, or at least 95%.

In one embodiment of the invention, the level of inhibition of target gene expression (i.e., mRNA expression) is at least 90%, at least 95%, at least 98%, at least 99% or is almost 100%, and hence the cell or organism will in effect have the phenotype equivalent to a so-called “knock out” of a gene. However, in some embodiments, it may be preferred to achieve only partial inhibition so that the phenotype is equivalent to a so-called “knockdown” of the gene. This method of knocking down gene expression can be used therapeutically or for research (e.g., to generate models of disease states, to examine the function of a gene, to assess whether an agent acts on a gene, to validate targets for drug discovery).

In certain embodiments, the moiety-coating polynucleotide nanoparticle using the compositions and methods of this present invention is synthesized as self-forming polynucleotide nanoparticle core, using techniques widely available in the art, then automatically surface formulated in-vitro, or extracellularly when in the presence of the aptamer-targeted surface moieties of the polynucleotide nanoparticle core.

In other embodiments, it is expressed in vitro or in vivo using appropriate and widely known techniques, then surface formulated in the same setting. Accordingly, in certain embodiments, the present invention includes in vitro and in vivo expression vectors or sequences comprising the sequence of a aptamer-containing self-forming polynucleotide nanoparticle, and candidate surface moieties used with the present invention. Methods well known to those skilled in the art may be used to construct expression vectors containing sequences encoding a self-forming polynucleotide nanoparticle, surface moieties, as well as appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

Expression vectors typically include regulatory sequences, which regulate expression of the self-forming polynucleotide nanoparticle. Regulatory sequences present in an expression vector include those non-translated regions of the vector, e.g., enhancers, promoters, 5′ and 3′ untranslated regions, which interact with host cellular proteins to carry out transcription and translation. Such elements may vary in their strength and specificity. Depending on the vector system and cell utilized, any number of suitable transcription and translation elements, including constitutive and inducible promoters, may be used. In addition, tissue- orcell specific promoters may also be used.

For expression in mammalian cells, promoters from mammalian genes or from mammalian viruses are generally preferred. In addition, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus that is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

In certain embodiments, the invention provides for the conditional expression of a candidate surface moiety polynucleotide nanoparticle. A variety of conditional expression systems are known and available in the art for use in both cells, plants, insect, and animals, and the invention contemplates the use of any such conditional expression system to regulate the expression or activity of a candidate target surface moiety. In one embodiment of the invention, for example, inducible expression of a target surface moiety is achieved using various inducible or tissue-preferred or developmentally regulated promoters.

A number of promoters can be used in the practice of the invention. The promoters can be selected based on the desired outcome. The nucleic acids can be combined with constitutive, tissue—preferred, inducible, or other promoters for expression in the host organism.

DNA Constructs for Expression in Plants

Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The disclosed polynucleotides also encompass all forms of sequences including, but not limited to, single-stranded forms, double-stranded forms, hairpins, stem-and-loop structures, and the like.

The polynucleotide encoding the self-forming polynucleotide nanoparticle core, with or without additional target surface moiety sequence elements, or in certain embodiments employed in the disclosed methods and compositions can be provided in expression cassettes for expression in a plant or organism of interest. In this embodiment, it is recognized that each nanoparticle core, or surface moiety, may be encoded by a single or separate cassette, DNA construct, or vector. As discussed, any means of providing the such elements is contemplated. A plant or plant cell can be transformed with a single cassette comprising DNA encoding one or elements or separate cassettes encoding a single element can be used to transform a plant or plant cell or host cell. Likewise, a plant transformed with one component can be subsequently transformed with the second component. One or more DNA constructs encoding single elements can also be brought together by sexual crossing. That is, a first plant comprising one component is crossed with a second plant comprising the second component. Progeny plants from the cross will comprise both components.

The expression cassette can include 5′ and 3′ regulatory sequences operably linked to the polynucleotide of this invention. “Operably linked” is intended to mean a functional linkage between two or more elements. For example, an operable linkage between a polynucleotide of the invention and a regulatory sequence (i.e., a promoter) is a functional link that allows for expression of the polynucleotide disclosed herein. Operably linked elements may be contiguous or non-contiguous. When used to refer to the joining of two protein coding regions, by operably linked is intended that the coding regions are in the same reading frame. The cassette may additionally contain at least one additional polynucleotide to be cotransformed into the organism. Alternatively, the additional polypeptide(s) can be provided on multiple expression cassettes. Expression cassettes can be provided with a plurality of restriction sites and/or recombination sites for insertion of the polynucleotide to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a polynucleotide encoding either the polynucleotide nanoparticle core which contains the moiety-targeting aptamers alone, or with transgenic candidate surface moieties, as employed in the methods and compositions of the invention, and a transcriptional and translational termination region (i.e., termination region) functional in plants.

The regulatory regions (i.e., promoters, transcriptional regulatory regions, and translational termination regions) and/or the polynucleotides disclosed herein may be native/analogous to the host cell or to each other. Alternatively, the regulatory regions and/or the polynucleotide disclosed herein may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The termination region may be native with the transcriptional initiation region, may be native with the operably linked polynucleotide encoding the polynucleotide nanoparticle compositions of this invention, may be native with the plant host, or may be derived from another source (i.e., foreign or heterologous) to the promoter, the polynucleotide encoding the polynucleotide compositions of this invention, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262: 141-144; Proudfoot (1991) Cell 64:671-674; Sanfacon et al. (1991) Genes Dev. 5: 141-149; Mogen et al. (1990) Plant Cell 2: 1261-1272; Munroe et al. (1990) Gene 91: 151-158; Ballas et al. (1989) Nucleic Acids Res. 17:7891-7903; and Joshi et al. (1987) Nucleic Acids Res. 15:9627-9639.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313: 810-812); rice actin (McElroy et al. (1990) Plant Cell 2: 163-171); ubiquitin (Christensen et al. (1989) Plant Mol. Biol. 12:619-632 and Christensen et al. (1992) Plant Mol. Biol. 18: 675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Depending on the desired outcome, it may be beneficial to express the gene from an inducible promoter. An inducible promoter, for instance, a pathogen-inducible promoter could also be employed. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4: 11 1-1 16. See also WO 99/43819.

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225: 1570-1573); WIP 1 (Rohmeier et al. (1993) Plant Mol. Biol. 22: 783-792; Eckelkamp et al. (1993) FEBS Letters 323:73-76); MPI gene (Corderok et al. (1994) Plant J. 6(2): 141-150); and the like.

Additionally, pathogen-inducible promoters may be employed in the methods and nucleotide constructs of the embodiments. Such pathogen-inducible promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89: 245-254; Uknes et al. (1992) Plant Cell 4: 645-656; and Van Loon (1985) Plant Mol. Virol. 4: 1 1 1-1 16. See also WO 99/43819.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93: 14972-14977. See also, Chen et al. (1996) Plant J. 10:955-966; Zhang et al. (1994) Proc. Natl. Acad. Sci. USA 91:2507-251 1; Warner et al. (1993) Plant J. 3: 191-201; Siebertz et al. (1989) Plant Cell 1:961-968; U.S. Pat. No. 5,750,386 (nematode-inducible). Of particular interest is the inducible promoter for the maize PRms gene, whose expression is induced by the pathogen Fusarium moniliforme (see, for example, Cordero et al. (1992) Physiol. Mol. Plant Path. 41: 189-200).

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88: 10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

Tissue-preferred promoters can be utilized to target enhanced expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 1 12(2):525-535; Canevascini et al. (1996) Plant Physiol. 1 12(2):513-524; Yamamoto et al. (1994) Plant Cell Physiol. 35(5):773-778; Lam (1994) Results Probl. Cell Differ. 20: 181-196; Orozco et al. (1993) Plant Mol Biol. 23(6): 1 129-1 138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6): 1 129-1 138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10): 1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1): 11-22 (full-length cDNA clone encoding cytosolic glutamine synthetase (GS), which is expressed in roots and root nodules of soybean). See also Bogusz et al. (1990) Plant Cell 2(7): 633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed roIC and roID root-inducing genes of Agrobacterium rhizogenes (see Plant Science (Limerick) 79(1): 69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and roIB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10: 108. Such seed-preferred promoters include, but are not limited to, CimI (cytokinin-induced message); cZ19B I (maize 19 kDa zein); and milps (myo-inositol-1-phosphate synthase) (see U.S. Pat. No. 6,225,529, herein incorporated by reference). Gamma-zein and Glob-1 are endosperm-specific promoters. For dicots, seed-specific promoters include, but are not limited to, bean □-phaseolin, nap in,□-conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc. See also WO 00/12733, where seed-preferred promoters from end1 and end2 genes are disclosed. A promoter that has “preferred” expression in a particular tissue is expressed in that tissue to a greater degree than in at least one other plant tissue. Some tissue-preferred promoters show expression almost exclusively in the particular tissue.

In an embodiment, the plant-expressed promoter is a vascular-specific promoter such as a phloem-specific promoter. A “vascular-specific” promoter, as used herein, is a promoter which is at least expressed in vascular cells, or a promoter which is preferentially expressed in vascular cells. Expression of a vascular-specific promoter need not be exclusively in vascular cells, expression in other cell types or tissues is possible. A “phloem-specific promoter” as used herein, is a plant-expressible promoter which is at least expressed in phloem cells, or a promoter which is preferentially expressed in phloem cells.

Expression of a phloem-specific promoter need not be exclusively in phloem cells, expression in other cell types or tissues, e.g., xylem tissue, is possible. In one embodiment of this invention, a phloem-specific promoter is a plant-expressible promoter at least expressed in phloem cells, wherein the expression in non-phloem cells is more limited (or absent) compared to the expression in phloem cells. Examples of suitable vascular-specific or phloem-specific promoters in accordance with this invention include but are not limited to the promoters selected from the group consisting of: the SCSV3, SCSV4, SCSV5, and SCSV7 promoters (Schunmann et al. (2003) Plant Functional Biology 30:453-60; the roIC gene promoter of Agrobacterium rhizogenes(Kiyokawa et al. (1994) Plant Physiology 104: 801-02; Pandolfini et al. (2003) BioMedCentral (BMC) Biotechnology 3:7, (www.biomedcentral.com/1472-6750/3/7); Graham et al. (1997) Plant Mol. Biol. 33:729-35; Guivarc'h et al. (1996); Almon et al. (1997) Plant Physiol. 1 15: 1599-607; the roIA gene promoter of Agrobacterium rhizogenes (Dehio et al. (1993) Plant Mol. Biol. 23: 1199-210); the promoter of the Agrobacterium tumefaciens T-DNA gene 5 (Korber et al. (1991) EM BO J. 10: 3983-91); the rice sucrose synthase RSs I gene promoter (Shi et al. (1994) J. Exp. Bot. 45:623-31); the CoYMV or Commelina yellow mottle badnavirus promoter (Medberry et al. (1992) Plant Cell 4: 185-92; Zhou et al. (1998) Chin. J. Biotechnol. 14: 9-16); the CFDV or coconut foliar decay virus promoter (Rohde et al. (1994) Plant Mol. Biol. 27:623-28; Hehn and Rhode (1998) J. Gen. Virol. 79: 1495-99); the RTBV or rice tungro bacilliform virus promoter (Yin and Beachy (1995) Plant J. 7:969-80; Yin et al. (1997) Plant J. 12: 1 179-80); the pea glutamin synthase GS3A gene (Edwards et al. (1990) Proc. Natl. Acad. Sci. USA 87:3459-63; Brears et al. (1991) Plant J. 1:235-44); the inv CD 1 1 1 and inv CD 141 promoters of the potato invertase genes (Hedley et al. (2000) J. Exp. Botany 51: 817-21); the promoter isolated from Arabidopsis shown to have phloem-specific expression in tobacco by Kertbundit et al. (1991) Proc. Natl. Acad. Sci. USA 88:5212-16); the VAHOX 1 promoter region (Tornero et al. (1996) Plant J. 9:639-48); the pea cell wall invertase gene promoter (Zhang et al. (1996) Plant Physiol. 1 12: 1 11 1-17); the promoter of the endogenous cotton protein related to chitinase of US published patent application 20030106097, an acid invertase gene promoter from carrot (Ramloch-Lorenz et al. (1993) The Plant J. 4:545-54); the promoter of the sulfate transporter gene, Sultrl; 3 (Yoshimoto et al. (2003) Plant Physiol. 131: 151 1-17); a promoter of a sucrose synthase gene (Nolte and Koch (1993) Plant Physiol. 101: 899-905); and the promoter of a tobacco sucrose transporter gene (Kuhn et al. (1997) Science 275-1298-1300).

Possible promoters also include the Black Cherry promoter for Prunasin Hydrolase (PH DL1.4 PRO) (U.S. Pat. No. 6,797,859), Thioredoxin H promoter from cucumber and rice (Fukuda A et al. (2005). Plant Cell Physiol. 46(1 1): 1779-86), Rice (RSs I) (Shi, T. Wang et al. (1994). J. Exp. Bot. 45(274): 623-631) and maize sucrose synthase-1 promoters (Yang., N-S. et al. (1990) PNAS 87:4144-4148), PP2 promoter from pumpkin Guo, H. et al. (2004) Transgenic Research 13:559-566), At SUC2 promoter (Truernit, E. et al. (1995) Planta 196(3):564-70., At SAM-1 (S-adenosylmethionine synthetase) (Mijnsbrugge K V. et al. (1996) Plant Cell. Physiol. 37(8): 1108-1 1 15), and the Rice tungro bacilliform virus (RTBV) promoter (Bhattacharyya-Pakrasi et al. (1993) Plant J. 4(I):71-79).

Where low level expression is desired, weak promoters will be used. Generally, the term “weak promoter” as used herein refers to a promoter that drives expression of a coding sequence at a low level. By low level expression at levels of about 1/1000 transcripts to about 1/100,000 transcripts to about 1/500,000 transcripts is intended. Alternatively, it is recognized that the term “weak promoters” also encompasses promoters that drive expression in only a few cells and not in others to give a total low level of expression. Where a promoter drives expression at unacceptably high levels, portions of the promoter sequence can be deleted or modified to decrease expression levels.

Such weak constitutive promoters include, for example the core promoter of the Rsyn7 promoter (WO 99/43838 and U.S. Pat. No. 6,072,050), the core 35S CaMV promoter, and the like. Other constitutive promoters include, for example, those disclosed in U.S. Pat. Nos. 5,608,149; 5,608,144; 5,604,121; 5,569,597; 5,466,785; 5,399,680; 5,268,463; 5,608,142; and 6,177,611.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 55:610-9 and Fetter et al. (2004) Plant Cell 7 (5:215-28), cyan florescent protein (CYP) (Bolte et al. (2004) J. Cell Science 777:943-54 and Kato et al. (2002) Plant Physiol 729:913-42), and yellow florescent protein (PhiYFP™ from Evrogen, see, Bolte et al. (2004) J. Cell Science 777:943-54). For additional selectable markers, see generally, Yarranton (1992) Curr. Opin. Biotech. 3:506-51 1; Christopherson al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90: 1917-1921; Labow et al. (1990) Mol. Cell. Biol. 10:3343-3356; Zambretti et al. (1992) Proc. Natl. Acad. Sci. USA 89:3952-3956; Bairn et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10: 143-162; Degenkolb et al. (1991) Antimicrob. Agents Chemother. 35: 1591-1595; Kleinschnidt et al. (1988) Biochemistry 27: 1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka ei l. (1985) Handbook of Experimental Pharmacology, Vol. 78 (Springer-Verlag, Berlin); Gill et al. (1988) Nature 334:721-724. The above list of selectable marker genes is not meant to be limiting. Any selectable marker gene can be used with the compositions and methods described herein.

Other components of this system and methods of using the system to control the expression of a gene are well documented in the literature, and vectors expressing the tetracycline-controlled transactivator (tTA) or the reverse tTA (rtTA) are commercially available (e.g., pTet-Off, pTet-On and ptTA-2/3/4 vectors, Clontech, Palo Alto, Calif.). Such systems are described, for example, in U.S. Pat. No. 5,650,298, No. 6271348, No. 5922927, and related patents, which are incorporated by reference in their entirety.

In one particular embodiment, surface-forming polynucleotide nanoparticles are expressed using a vector system comprising a pSUPER vector backbone and additional sequences corresponding to the self-forming polynucleotide nanoparticle to be expressed. The pSUPER vectors system has been shown useful in expressing siRNA reagents and downregulating gene expression (Brummelkamp, T. T. et al., Science 296:550 (2002) and Brummelkamp, T. R. et al., Cancer Cell, published online Aug. 22, 2002). PSUPER vectors are commercially available from OligoEngine, Seattle, Wash.

The aptamer-driven surface forming polynucleotide nanoparticles of the invention may be used for a variety of purposes, all generally related to their ability to efficiently deliver into target cells a polynucleotide nanoparticle to inhibit or reduce expression of a target gene. Accordingly, the invention provides methods of reducing expression of one or more target genes comprising introducing a self-forming polynucleotide nanoparticle of the invention into a cell that contains a target gene or a homolog, variant or ortholog thereof. In addition, self-forming moiety-coated polynucleotide nanoparticles may be used to reduce expression indirectly. For example, a self-forming moeity-coated polynucleotide nanoparticle may be used to reduce expression of a transactivator that drives expression of a second gene, thereby reducing expression of the second gene. Similarly, a self-forming moiety-coated polynucleotide nanoparticle may be used to increase expression indirectly. For example, a self-forming moiety-coated polynucleotide nanoparticle may be used to reduce expression of a transcriptional repressor that inhibits expression of a second gene, thereby increasing expression of the second gene.

In various embodiments, a target gene is a gene derived from the cell into which a self-forming moiety-coated polynucleotide nanoparticle is to be introduced, an endogenous gene, an exogenous gene, a transgene, or a gene of a pathogen that is present in the cell after transfection thereof. Depending on the particular target gene and the amount of the self-forming moiety-coated polynucleotide nanoparticle delivered into the cell, the method of this invention may cause partial or complete inhibition of the expression of the target gene. The cell containing the target gene may be derived from or contained in any organism (e.g., plant, animal, protozoan, virus, bacterium, or fungus).

Inhibition of the expression of the target gene can be verified by means including, but not limited to, observing or detecting an absence or observable decrease in the level of protein encoded by a target gene, and/or mRNA product from a target gene, and/or a phenotype associated with expression of the gene, using techniques known to a person skilled in the field of the present invention.

Examples of cell characteristics that may be examined to determine the effect caused by introduction of a self-forming moiety-coated polynucleotide nanoparticle of the invention include, cell growth, apoptosis, cell cycle characteristics, cellular differentiation, and morphology.

A self-forming moiety-coated polynucleotide nanoparticle may be directly introduced to the cell (i.e., intracellularly), or introduced extracellularly into a cavity, interstitial space, into the circulation of an organism, introduced orally, by ingestion of the expression host, by bathing an organism in a solution containing the self-forming moiety-coated polynucleotide nanoparticle, or by some other means sufficient to deliver the self-forming moiety-coated polynucleotide nanoparticle into the cell.

In addition, a vector engineered to express a self-forming polynucleotide nanoparticle may be introduced into a cell, wherein the vector expresses the self-forming polynucleotide nanoparticle, thereby introducing it into the cell. Methods of transferring an expression vector into a cell are widely known and available in the art, including, e.g., transfection, lipofection, scrape loading, electroporation, microinjection, infection, gene gun, and retrotransposition. Generally, a suitable method of introducing a vector into a cell is readily determined by one of skill in the art based upon the type of vector and the type of cell, and teachings widely available in the art. Infective agents may be introduced by a variety of means readily available in the art, including, e.g., nasal inhalation.

Methods of inhibiting gene expression using self-forming moiety-coated polynucleotide nanoparticles of the invention may be combined with other knockdown and knockout methods, e.g., gene targeting, antisense RNA, ribozymes, double-stranded RNA (e.g., shRNA and siRNA) to further reduce expression of a target gene.

In different embodiments, target cells of the invention are primary cells, cell lines, immortalized cells, or transformed cells. A target cell may be a somatic cell or a germ cell. The target cell may be a non-dividing cell, such as a neuron, or it may be capable of proliferating in vitro in suitable cell culture conditions. Target cells may be normal cells, or they may be diseased cells, including those containing a known genetic mutation. Eukaryotic target cells of the invention include mammalian cells, such as, for example, a human cell, a murine cell, a rodent cell, and a primate cell. In one embodiment, a target cell of the invention is a stem cell, which includes, for example, an embryonic stem cell, such as a murine embryonic stem cell.

The self-forming shell-forming polynucleotide nanoparticles and methods of the present invention may be used for regulating genes in plants, e.g., by providing RNA for systemic or non-systemic regulation of genes.

The self-forming shell-forming polynucleotide nanoparticles and methods of the present invention are useful for regulating endogenous genes of a plant pest or pathogen.

The self-forming shell-forming polynucleotide nanoparticles and methods of the present invention may be used to treat any of a wide variety of diseases or disorders, including, but not limited to, inflammatory diseases, cardiovascular diseases, nervous system diseases, tumors, demyelinating diseases, digestive system diseases, endocrine system diseases, reproductive system diseases, hemic and lymphatic diseases, immunological diseases, mental disorders, musculoskeletal diseases, neurological diseases, neuromuscular diseases, metabolic diseases, sexually transmitted diseases, skin and connective tissue diseases, urological diseases, and infections.

In certain embodiments, the methods are practiced on an animal, in particular embodiments, a mammal, and in certain embodiments, a human.

Accordingly, in one embodiment, the present invention includes methods of using a self-forming shell-forming polynucleotide nanoparticle for the treatment or prevention of a disease associated with gene deregulation, overexpression, or mutation. For example, a self-forming polynucleotide nanoparticle may be introduced into a cancerous cell or tumor and thereby inhibit expression of a gene required for or associated with maintenance of the carcinogenic/tumorigenic phenotype. To prevent a disease or other pathology, a target gene may be selected that is, e.g., required for initiation or maintenance of a disease/pathology. Treatment may include amelioration of any symptom associated with the disease or clinical indication associated with the pathology.

In addition, self-forming shell-forming polynucleotide nanoparticles of the present invention are used to treat diseases or disorders associated with gene mutation. In one embodiment, a self-forming polynucleotide nanoparticle is used to modulate expression of a mutated gene or allele. In such embodiments, the mutated gene is the target of the self-forming polynucleotide nanoparticle, which will comprise a region complementary to a region of the mutated gene. This region may include the mutation, but it is not required, as another region of the gene may also be targeted, resulting in decreased expression of the mutant gene or mRNA. In certain embodiments, this region comprises the mutation, and, in related embodiments, the resulting self-forming shell-forming polynucleotide nanoparticles specifically inhibits expression of the mutant mRNA or gene but not the wild type mRNA or gene. Such a self-forming polynucleotide nanoparticle is particularly useful in situations, e.g., where one allele is mutated but another is not. However, in other embodiments, this sequence would not necessarily comprise the mutation and may, therefore, comprise only wild-type sequence. Such a self-forming polynucleotide nanoparticle is particularly useful in situations, e.g., where all alleles are mutated. A variety of diseases and disorders are known in the art to be associated with or caused by gene mutation, and the invention encompasses the treatment of any such disease or disorder with a self-forming polynucleotide nanoparticle.

In certain embodiments, a gene of a pathogen is targeted for inhibition. For example, the gene could cause immunosuppression of the host directly or be essential for replication of the pathogen, transmission of the pathogen, or maintenance of the infection. In addition, the target gene may be a pathogen gene or host gene responsible for entry of a pathogen into its host, drug metabolism by the pathogen or host, replication or integration of the pathogen's genome, establishment or spread of an infection in the host, or assembly of the next generation of pathogen. Methods of prophylaxis (i.e., prevention or decreased risk of infection), as well as reduction in the frequency or severity of symptoms associated with infection are included in the present invention. For example, cells at risk for infection by a pathogen or already infected cells, particularly human immunodeficiency virus (HIV) infections, may be targeted for treatment by introduction of a self-forming polynucleotide nanoparticle according to the invention.

In other specific embodiments, the present invention is used for the treatment or development of treatments for cancers of any type. Examples of tumors that can be treated using the methods described herein include, but are not limited to, neuroblastomas, myelomas, prostate cancers, small cell lung cancer, colon cancer, ovarian cancer, non-small cell lung cancer, brain tumors, breast cancer, leukemias, lymphomas, and others.

The self-forming shell-forming polynucleotide nanoparticles and expression vectors (including viral vectors and viruses) may be introduced into cells in vitro or ex vivo and then subsequently placed into an animal to affect therapy, or they may be directly introduced to a patient by in vivo administration. Thus, the invention provides methods of gene therapy, in certain embodiments. Compositions of the invention may be administered to a patient in any of a number of ways, including parenteral, intravenous, systemic, local, oral, intratumoral, intramuscular, subcutaneous, intraperitoneal, inhalation, or any such method of delivery. In one embodiment, the compositions are administered parenterally, i.e., intraarticularly, intravenously, intraperitoneally, subcutaneously, or intramuscularly. In a specific embodiment, the liposomal compositions are administered by intravenous infusion or intraperitoneally by a bolus injection.

Compositions of the invention may be formulated as pharmaceutical compositions suitable for delivery to a subject. The pharmaceutical compositions of the invention will often further comprise one or more buffers (e.g., neutral buffered saline or phosphate buffered saline), carbohydrates (e.g., glucose, mannose, sucrose, dextrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The amount of self-forming shell-forming polynucleotide nanoparticles administered to a patient can be readily determined by a physician based upon a variety of factors, including, e.g., the disease and the level of self-forming shell-forming polynucleotide nanoparticles expressed from the vector being used (in cases where a vector is administered). The amount administered per dose is typically selected to be above the minimal therapeutic dose but below a toxic dose. The choice of amount per dose will depend on a number of factors, such as the medical history of the patient, the use of other therapies, and the nature of the disease. In addition, the amount administered may be adjusted throughout treatment, depending on the patient's response to treatment and the presence or severity of any treatment-associated side effects.

The invention further includes a method of identifying gene function in an organism comprising the use of a self-forming polynucleotide nanoparticle to inhibit the activity of a target gene of previously unknown function. Instead of the time consuming and laborious isolation of mutants by traditional genetic screening, functional genomics envisions determining the function of uncharacterized genes by employing the invention to reduce the amount and/or alter the timing of target gene activity. The invention may be used in determining potential targets for pharmaceutics, understanding normal and pathological events associated with development, determining signaling pathways responsible for postnatal development/aging, and the like. The increasing speed of acquiring nucleotide sequence information from genomic and expressed gene sources, including total sequences for the yeast, D. melanogaster, and C. elegans genomes, can be coupled with the invention to determine gene function in an organism (e.g., nematode). The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects.

In one embodiment, a self-forming polynucleotide nanoparticle is used to inhibit gene expression based upon a partial sequence available from an expressed sequence tag (EST), e.g., in order to determine the gene's function or biological activity. Functional alterations in growth, development, metabolism, disease resistance, or other biological processes would be indicative of the normal role of the EST's gene product.

The ease with which a self-forming polynucleotide nanoparticle can be introduced into an intact cell/organism containing the target gene allows the present invention to be used in high throughput screening (HTS). For example, solutions containing self-forming polynucleotide nanoparticle that are capable of inhibiting different expressed genes can be placed into individual wells positioned on a microtiter plate as an ordered array, and intact cells/organisms in each well can be assayed for any changes or modifications in behavior or development due to inhibition of target gene activity. The function of the target gene can be assayed from the effects it has on the cell/organism when gene activity is inhibited. In one embodiment, self-forming shell-forming polynucleotide nanoparticles of the invention are used for chemocogenomic screening, i.e., testing compounds for their ability to reverse a disease modeled by the reduction of gene expression using a self-forming polynucleotide nanoparticle of the invention.

If a characteristic of an organism is determined to be genetically linked to a polymorphism through RFLP or QTL analysis, the present invention can be used to gain insight regarding whether that genetic polymorphism may be directly responsible for the characteristic. For example, a fragment defining the genetic polymorphism or sequences in the vicinity of such a genetic polymorphism can be amplified to produce an RNA, a self-forming polynucleotide nanoparticle can be introduced to the organism, and whether an alteration in the characteristic is correlated with inhibition can be determined.

The present invention is also useful in allowing the inhibition of essential genes. Such genes may be required for cell or organism viability at only particular stages of development or cellular compartments. The functional equivalent of conditional mutations may be produced by inhibiting activity of the target gene when or where it is not required for viability. The invention allows addition of a self-forming polynucleotide nanoparticle at specific times of development and locations in the organism without introducing permanent mutations into the target genome. Similarly, the invention contemplates the use of inducible or conditional vectors that express a self-forming polynucleotide nanoparticle only when desired.

The present invention also relates to a method of validating whether a gene product is a target for drug discovery or development. A self-forming polynucleotide nanoparticle that targets the mRNA that corresponds to the gene for degradation is introduced into a cell or organism. The cell or organism is maintained under conditions in which degradation of the mRNA occurs, resulting in decreased expression of the gene. Whether decreased expression of the gene has an effect on the cell or organism is determined. If decreased expression of the gene has an effect, then the gene product is a target for drug discovery or development.

Methods of Designing Moiety-Coated Polynucleotide Nanoparticles

According to certain embodiments, a method for designing moiety-coated polynucleotide nanoparticles is provided herein. Such methods include steps of (i) designing nanoparticle core units, selecting and producing surface moiety material, designing the nanoparticle core, characterizing a simulated nanoparticle, determining the number of aptamers for each surface moiety, assembling the final nanoparticle sequence, verifying nanoparticle folding with final aptamer(s), and producing the nanoparticle(s).

STEP I: Design Nanoparticle Core Units. The first step of the method is to design and screen efficacious MV-RNA according to methods in U.S. Pat. No. 9,200,276. Use ‘RNAi Cloud’ software to design all MV-RNA candidates for any number of gene targets. Alternatively, design hairpins or MV-RNA structures with or without screening for RNAi activity when only surface MOA is needed. In certain embodiments, it is suggested that a minimum of 2 different MV-RNA structures are used in plurality to form the polynucleotide nanoparticle core-not including aptamers optionally placed in-between each core unit.

STEP II: Select and Produce Surface Moiety Material. The second step of the method is to determine the composition of exogenous or endogenous moieties desired as a surface coating for the nanoparticles, such as specific peptides, proteins, polymers, metabolites, ions, small molecules, oligosaccharides, or other organic or inorganic moieties—non-limiting examples of which are provided above. This determination can be made according to the following substeps:
1. Calculate each candidate moiety MW, net charge at final desired pH.
2. Produce surface moiety material accordingly for downstream SELEX or other aptamer selecting experiments.
3. Produce simulation material to determine optimized molarity/number of aptamers as follows in steps 4-5.
4. If peptides, produce additional pre-screening surface peptides accordingly with N or C Terminus modification for simulations;
   (a) Electrostatic N Terminus additions;
      Histidine/lysine copolymer: “KHKHKHKHKHKHKHKHKH”
      Arginine Frag: “RRRRRRRRR”
   (b) Pseudo-aptamer N or C Terminus additions;
      Any specific peptide fragment to a known aptamer (i.e., Tat, BIV, Rev1, Rev2)
5. If protein, produce additional pre-screening surface proteins with TAG for aptamer binding modification for simulation;
   (a) Pseudo-aptamer N or C Terminus additions;
      Any specific peptide fragment to a known aptamer (i.e., Tat, Rev1, BIV, Rev2)

STEP III: Design Nanoparticle Core. The third step of the method is to design the nanoparticle core according to design the nanoparticle core according to PCT/US16/48492 using results from STEP I. In certain embodiments, hairpins can be used in place of MV-RNA, but MV-RNA or 3-way junction containing constructs are a preferred embodiment to accommodate the plurality of moiety-binding aptamers. For a simulation version using a pseudo-aptamer or with aptamers specifically designed to directly bind moieties without additional aa, aptamer(s)/intramer(s) sequences may be inserted into the nanoparticle sequence for simulation nanoparticle according to compositions of FIG. 4.

STEP IV: Characterization of the simulated nanoparticle. The fourth step of the method is to test and characterize the function of each potential surface moiety. For example, it is fundamental to run titrations of each potential surface moiety and test function in the intended setting. One such method to determine final surface moiety composition and number of aptamer/intramer(s) is to use either a electrostatic or pseudo-aptamer model as directed in STEP II, substeps 4-5. The use of pseudo-aptamers is preferable over electrostatic-binding alone due to the advantages of this patent are in use when aptamer-driven binding forces are used in surface formation. The following substeps can be used to characterize the simulated nanoparticle:
1. Setup titrations to test for surface changes are various concentrations or N/P ratios in a buffer that most resembles final salt composition and pH. One starting point for the molar ratios according to the embodiments described herein are 4,8,16,32,64, and 128 for any individual surface moiety, or entire pool when a mix of surface moieties is simulated. Each reaction should sit at room temperature for ˜30 minutes or longer before measurements.
2. Measure Zeta Potential for each titration and composition.
3. A gel shift or fluorophore inhibition assay is an easy confirmation of binding and surface charge changes.
4. Measure nanoparticle size and PI for each titration.
5. Test activity/function for each titration and composition.

STEP V: Determine Number of Aptamers for Each Surface Moiety. Based on characterization in STEP IV, particular surface moieties and the quantity of each moiety per nanoparticle is suggested. The results may indicate how many MV-RNA should be in your nanoparticle transcript to and the number of aptamers required to achieve your desired characteristics. (i.e., If 24 surface units are required for your desired outcome, a minimum of 12 MV-RNA or 3-way junction aptamers would have to be included in your nanoparticle transcript. Next, SELEX-like assays are run for each aptamer/intramer(s) for each moiety.

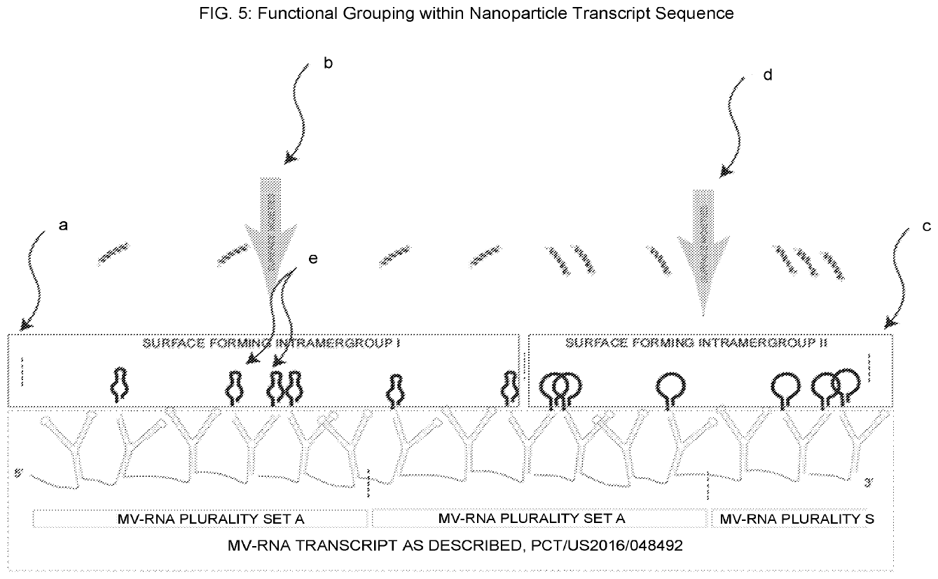

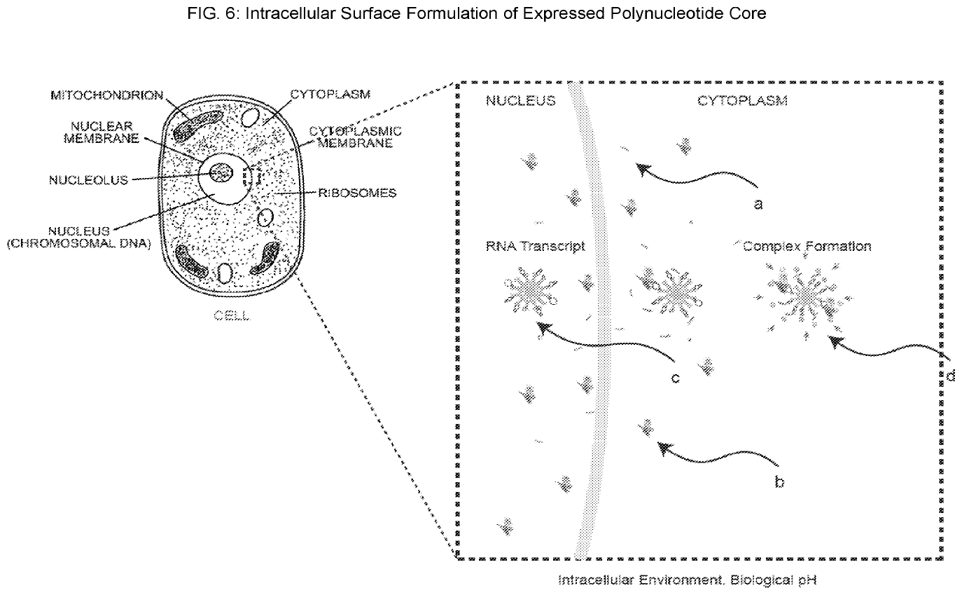

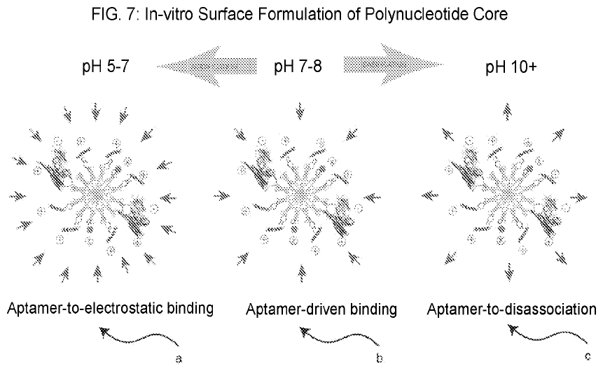

STEP VI: Assemble Final Nanoparticle Sequence. For the sixth step of the method, aptamer sequences from Step 4 are arranged within the loops or as stem-loops (FIG. 4) of the nanostructure of STEP 2, grouped or ungrouped (FIG. 5), as preferred.

STEP VII: Verifying Nanoparticle Folding with Final Aptamers. Once the full sequence of each MV-RNA is designed and oriented into a nanostructure with aptamer/intramer sequences using one of the patterns of FIG. 5, re-fold the RNA in a computer program like cofold. Folding software based on thermodynamics may not provide as accurate of results as cofold-transcription based folding. Alternatively, one may use ‘RNAi Cloud’ software to automatically design the MV-RNA units, select from a library of aptamers for pseudo or direct moiety binding, and verify the resulting secondary structure of nanoparticles.

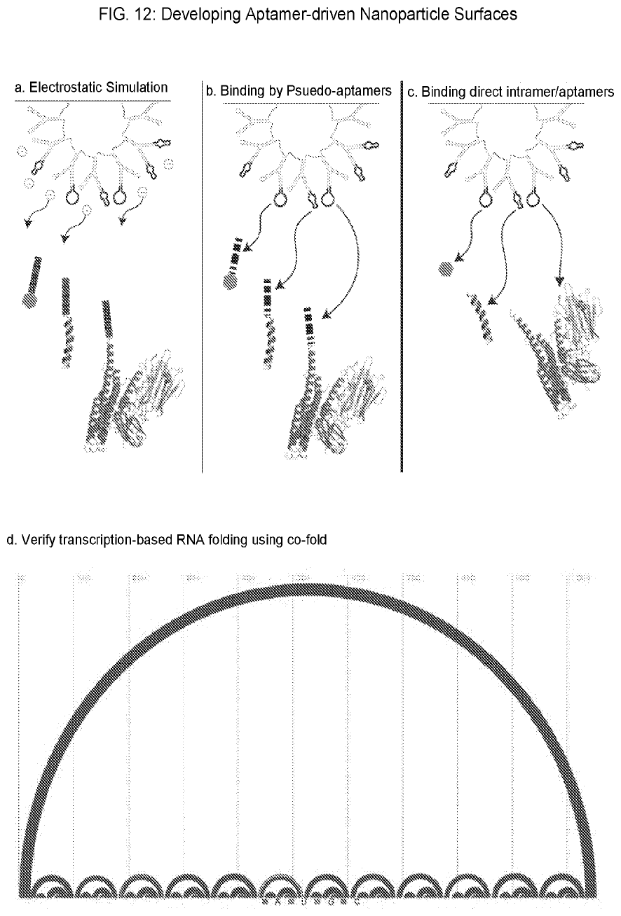

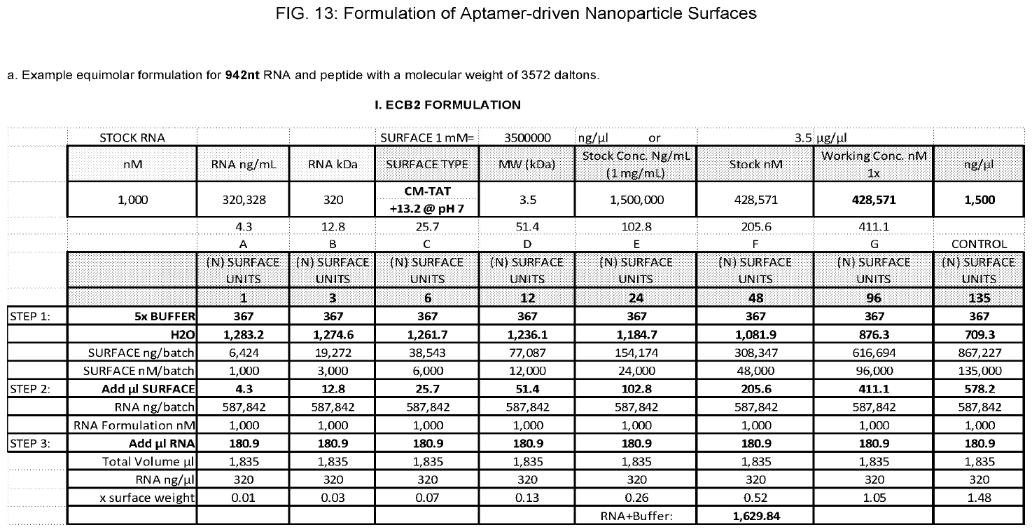

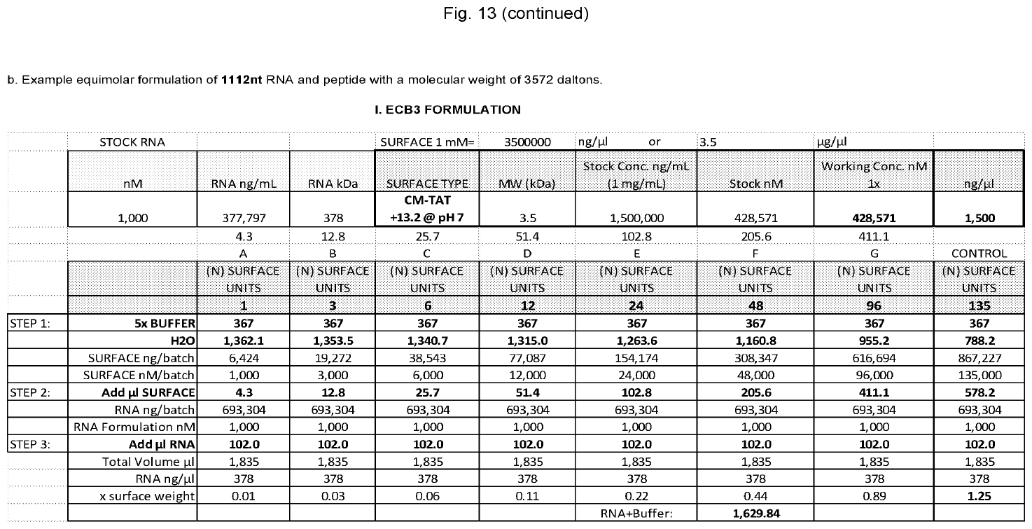

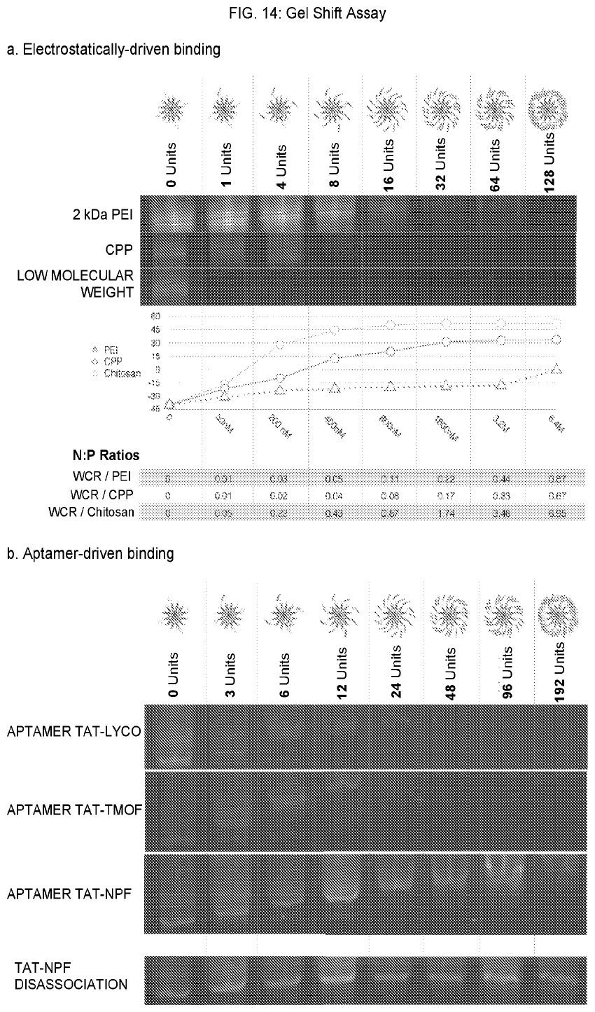

The resulting fold notation or art will indicate free nucleotides as “.” and bound nucleotides as “(” or “)”. Relative Free-energy and melting temperature will also give indication as to the stability of the precise transcript. One can view the resulting art representing the precisely structured transcript. An exemplar co-fold notation of the sequences shown in FIGS. 12d, 14 is shown below:

((((((((((((((((((((((((((((((.(((((((..))))))).(((((((((((...((((......))))))))))))))))))))))))...((((((((.(((((((((....)))))))))((((((((((...((((......))))))))))))).))))))))...((.((((((.(((((((((....)))))))))((((((( ((((...((((......))))))))))))))..)))))).))..(((((((((.(((((((((....))))))))).(((((((((((...((((......)))))))))))))) .)))))))))..((((((((((..(((((((((....)))))))))(((((((((...((((......)))))))))))))).))))))))))..(..((((((..(((((((((....))))))))).((((((((((...((((......)))))))))))))..))))))..)..(((((((((..(((((((((....)))))))))(((((((((((...(((( .. ....))))))))))))))).)))))))))..(((((((((...(((((((((....)))))))))(((((((((((...((((......)))))))))))))).)))))))))..(((((((((...(((((((((....))))))))).(((((((((...((((......)))))))))))))).)))))))))...((((((((.....(((((((((....)))) )) )))(((((((((((...((((......)))))))))))))).))))))))...((((((((....(((((((((....)))))))))(((((((((((...((((......)) )))))))))))))))))))))).((((((((((.(((((((((....)))))))))(((((((((...((((......))))))))))))))..)))))))))).))))))) )))))))))))))..

STEP VIII: Nanoparticle Production. For the eighth step, the sequence is ready to be incorporated into the appropriate transcriptional setting. One skilled in the art would understand how to select the appropriate transcription promoter and termination motif for in-vitro or intracellular production.

These methods, in certain embodiments, include determining or predicting the secondary structure adopted by the sequences selected in step (b), e.g., in order to determine that they are capable of adopting a stem-loop, or 3-way junction structure.

Similarly, these methods can include a verification step, which comprises testing the designed polynucleotide sequence for its ability to inhibit expression of a target gene, e.g., in an in-vivo or in-vitro test system.

The invention further contemplates the use of a computer program to select MV-RNA sequences of the nanoparticle, based upon the complementarity characteristics described herein. The invention, thus, provides computer software programs, and computer readable media comprising said software programs, to be used to select the polynucleotide nanoparticle sequences, as well as computers containing one of the programs of the present invention.

In certain embodiments, a user provides a computer with information regarding the sequences, locations or names of the target gene(s). The computer uses this input in a program of the present invention to identify one or more appropriate regions of the target gene to target in MV-RNA formats, and outputs or provides complementary sequences to use for the assembly of a polynucleotide nanoparticle of the invention according to the embodiments described herein. Typically, the program will select a series of sequences that are not complementary to a genomic sequence, including the target gene, or the region of the polynucleotide nanoparticle that is complementary to the target gene. When desired, the program also provides sequences of gap regions, fold notations, and fold art. Upon selection of appropriate MV-RNA orientations, plurality, aptamers, loops, linkages, Opening/Closing sequence, cloning sites, and necessary transcription elements, the computer program outputs or provides this information to the user.

The programs of the present invention may further use input regarding the genomic sequence of the organism containing the target gene, e.g., public or private databases, as well as additional programs that predict secondary structure and/or hybridization characteristics of particular sequences, in order to ensure that the polynucleotide nanoparticle adopts the correct secondary structure (i.e., mFold, RNAfold, cofold, RNAi Cloud) and does not hybridize to non-target genes (BLASTn).

The present invention is based, in part, upon the surprising discovery that only a very small amount of surface material is needed to dramatically alter the surface measurements of these polynucleotide nanoparticle cores and that such surface material can be controlled by aptamer/intramer binding at levels low enough to enable intracellular formulation, as described herein, and is extremely effective at expanding the utility of polynucleotide nanoparticles. The resulting moiety-coated polynucleotide nanoparticles of this invention offer significant advantages over previously described techniques, including a first-of-its-kind intracellular formulation method controlling surface composition, surface charge, N/P ratio, stability, cellular uptake, transport, mode of action. Furthermore, the core/shell polynucleotide nanoparticles of the invention offer additional advantages over traditional biomolecules by potentially utilizing thousands coating moieties, in nearly a limitless mixture of compositions tunable for a given use, resulting in novel polar, or non-polar, or amphipathic molecules-with multimodal, and multivalent modes of action-intracellularly, extracellularly, in-vitro, or in-vivo.

The practice of the present invention will employ a variety of conventional techniques of cell biology, molecular biology, microbiology, and recombinant DNA, which are within the skill of the art. Such techniques are fully described in the literature. See, for example, Molecular Cloning: A Laboratory Manual, 2nd Ed., ed. by Sambrook, Fritsch, and Maniatis (Cold Spring Harbor Laboratory Press, 1989); and DNA Cloning, Volumes I and II (D. N.

### WORKING EXAMPLES

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1: Aptamer-driven Surfaces to Alter Pharmacodynamics of a Polynucleotide Nanoparticle Targeting Pests

In certain embodiments, the methods and compositions provided herein may be used for agricultural purposes. As described herein, the invention can be applied to a polynucleotide nanoparticle contributing to additional modes of action, cellular recognition and/or uptake in a highly specific manner. For agricultural uses, such as crop protection, the approaches are not only a useful strategy to improve upon the bioavailability of polynucleotide nanoparticles, but to provide additional modes of action.

This example describes the assembly of an aptamer-driven nanoparticle surface sequence according to the invention. The programmable surface composition is enabled by a plurality of intramer/aptamer sequences specifically designed for the low-molar binding of targeted moieties onto the surface of the polynucleotide nanoparticle. The single-stranded self-forming polynucleotide nanoparticle core delivers an array of active RNAi triggers targeting genes in European corn borer. With the addition of this invention, the core polynucleotide sequence is supplemented with a plurality of aptamer/intramer sequences which bind targeted moieties onto the nanoparticle core's surface forming a shell-like surface. The formed surface presents characteristics atypical of polynucleotides and/or additional function.

This non-limiting example illustrates the binding of peptide insect toxins as the nanoparticle surface, and a method to expedite the development of aptamer-driven surfaces using pseudo-peptide/aptamer [FIG. 12b] relationships for assays.

Preparation of a Polynucleotide Nanoparticle Sequence

In this nanoparticle core example, two genes are targeted: (1) chitinase, and (2) neuropeptide. Multiple nanoparticle core sequences were assembled with various RNAi trigger pluralities according to [Hauser, PCT/US2016/048492]. The ECB target genes are shown below:

>GU329524.1 Ostrinia nubilalis chitinase (Cht)
mRNA, complete cds
(SEQ ID NO: ______)
GGGCTTCACTATGGGATTGATTTTATTATTTGTGTTGGGTTTTTGTGCCA
GTGCGGTGTTCGCGAATGACGATAAAATAGTAGTCTGCTACTATGGCACA
TGGGCGACATACCGGACTGGCTTGGGCAAGTTCGACGTAGACGACATCGA
CCCATTCCTCTGCACGCATCTGGTATATGCCTTCATCGGCATTAATGCTG
AAGGAACAGCTCTGGCGCTTGACCCTGAGCTTGATGTTGAGAGAGGTAAC
TTCAAGAACTTCACTTCGTTGAAAGAAAAGAACCCGAACCTCAAAACGCT
TGTAGCTGTCGGAGGATGGAGCGAAGGATCAGCTAATTACTCTATTATGG
CAGCAGAACCAGAATATCGGCAGAACTTCATCAACACCTCTCTGGCAATG
ATACTGGAATACAACTTCGATGGTCTGGACGTAGACTGGGAGTACCCCAA
TCGCAGAGACACGGTGCATGGTGAAGACGACATTGAGAACTTCAGCACTC
TCCTTAAGGAGTTGAGGGAGGAATTCGATAATTACGGTCTACTGCTGACA
GTCGCTGTGGCTGCTGTGGAGGAAGCTGCTGTGCAGTCCTATGACGTTCC
AAGTGTTGCCAAGTACGTAGACTACATTGGCGTCATGACGTATGACATGC
ACGGCGCCTGGGACTCCGTGACCGGCCACAACGCGCCTCTGTTCATAAGT
GAAGGCGAGAGTGCTGAAAACGAAAGCACTCTGTACAACGTCAATAACGC
TGTCCAGTATTGGCTTAGTGCAGGATGTCCTCCCGAGAAGTTAGTTATGG
GGGTTCCATTCTATGGGCGCACTTTCAACCTGAGTGACCCTTCAGTTAAC
GCTCCAAATTCACCTTCCAACGGAGCTGGTCTCGCCGGTCCTTACACTGC
TGAAAGTGGATTTATCGGGTATAATGAGTTCTGCTATATTCTCCAGAACG
AGTCTTCTTGGACCGTCCAGACCGACAACCTTGCCAAAGTGCCTTACGCC
TTCCTCGACTACAACTGGGTATCTTTCGATAACGTTGAGTCCATGACCGC
CAAAGTGGAGTACGCTAACAGCTTCAACCTTCGCGGCATCATGCTCTGGA
GTATCGAGACTGATGACTTCCACGGCCTCTGTGGAGAGGGAACATTCCCT
CTGCTGAACACCATCAACACAGTTCTAGCTGAAGGCTCAACAGAAGCCAG
ACACAACAACCCTCCTCATCATCACCATCATTAAATATGGAAACCTACTT
CCAGGTCCTTAAAAGAAGAATTAAAGACAAATCCATAGTACAAGTATTAT
GAATCTTCCAGAGTGCCAGGAGACGGCACTCTTTTCATAATTAACACCAA
AAACGAGGCAAAGATAATAAAAAATATATTTCTGCAAAAAAAAAAAAAAA
AAAA
>ENA|HM159463|HM159463.1 Ostrinia nubilalis
neuropeptide F1 (NPF1) mRNA, complete cds.:
Location: 1 . . . 894
(SEQ ID NO: ______)
GCACGAGGGCAACTCGACCTACCAAGTTTGAAAAGTTCGCCGCGCGTTCC
GCACATCTATCCGCATCCAAGATGATGAGCAAGAGAATCGCCGTCGCCAT
CGCCGTGGTCCTGGCCGTGATCTGCTTGGCTGAGGCCAGGGAGGAGGGTC
CTCACGACATGGCCGACGCCCTCCGTATGCTCCAGGAACTCGACCGCTAC
TACACTCAAGCTGCTAGACCGAGGTTCGGCAAGCGATCGGATGCGTTCAC
GAACTGGGCTAAGGATCTCGAAAAGCCGGACTTACCCGCTTGGTTGGCGT
ACGCCAGAAGGAGATGAGGGGCGCAACAGCTGCCGGGTCTATAAGTCACT
ATCAATGTTCATGATCACTACACTATGTACCGGACTCAATGTTAATACCG
TTAATATTACACCTCTTTCTGTCGTTCATGTCACACTCGTTGTCTCTGTT
ATCATGGTCCAAATTCAAATTTTTGAAATATAAAAAGTTACTTTTTAATT
TAGATAGATATAGATTTCATTTAGAAGTTTACTTTTTAAATGTTTAAATG
TTTTCCATTTCAATAAATAAATAGGTTGATTGAAAATAGGCTGGCATTTC
AATCTCTTCTATTGCTGTGGTGCTTTTGTACTTAGGTGTTTATCTTTATT
TTAATTTGTGAAATTCTAGTCAAAACGATAGCATTTCTGTGCTATCTTCT
GAAAACAATTTGTGCCAAAGTTAACTTTTACTTTTACAAAAAAATGATTT
TTTGGAAAAACCTCTCGATTCAAAGTATTTTAATATTATTGTTTCTATAA
TGTAACCCAGTCGATCAATCCCATAGTCATTAGTATTCATAGAATCTTTT
CTGTAACTTTAAGAATTATTATGTCATAATATTTGAACCAAAAT

The individual MV-RNA designs are derived from the software application ‘RNAi Cloud’ projects #P01059, entitled ‘ECB Neuropeptide F1 (NPF1)’ and #P01058, entitled ‘ECB Chitinase’.

A few of the MV-RNA designs were selected based on the confidence ranking in the RNAi Cloud software. Each set of MV-RNA were then grouped and linked as instructed. The results are as follows:

Chitinase Divalent MV-RNA:

288/874,
(SEQ ID NO: ______)
CGUUGGAAGGUGAAUUUGGUUCCAAAUUCAAAAGA
ACCCUUCAAGUGGGUUCUUUUCUUUCAACGuu
249/1355,
(SEQ ID NO: ______)
UUUUGGUGUUAAUUAUGAAAuaUUUCAUAAUUGA
GAGAGGUUCAAGUCCUCUCUCAACAUCAAGCuu
750/743,
(SEQ ID NO: ______)
ACGUUGUACAGAGUGCUUUUauAAAAGCACUC
CGUCAAUAAUUCAAGUUUAUUGACGUUGUACAGAGUuu
348/341,
(SEQ ID NO: ______)
AAUUAGCUGAUCCUUCGCUGUUCAGCGAAGGA
UUUACUCUAUUUCAAGUAUAGAGUAAUUAGCUGAUUuu
79/69,
(SEQ ID NO: ______)
CAUUCGCGAACACCGCACUGGUUUACCAGUG
CGGGACGAUAAAUUCAAGUUUUAUCGUCAUUCGCGAAUG
uu
227/364,
(SEQ ID NO: ______)
CUGGUUCUGCUGCCAUAAUAUUUAUAUUAU
GGCCGCUUGACCUUCAAGUGGUCAAGCGCCAGAGCUGGu
u

Chitinase set linked as ssRNA:

(SEQ ID NO: ______)
CGUUGGAAGGUGAAUUUGGUUCCAAAUUCAAAAGAACCCU
UCAAGUGGGUUCUUUUCUUUCAACGuuUUUUGGUGUUAAU
UAUGAAAuaUUUCAUAAUUGAGAGAGGUUCAAGUCCUCUCU
CAACAUCAAGCuuACGUUGUACAGAGUGCUUUUauAAAAGC
ACUCCGUCAAUAAUUCAAGUUUAUUGACGUUGUACAGAGUu
uAAUUAGCUGAUCCUUCGCUGUUCAGCGAAGGAUUUACUC
UAUUUCAAGUAUAGAGUAAUUAGCUGAUUuuCAUUCGCGAA
CACCGCACUGGUUUACCAGUGCGGGACGAUAAAUUCAAGU
UUUAUCGUCAUUCGCGAAUGuuCUGGUUCUGCUGCCAUAA
UAUUUAUAUUAUGGCCGCUUGACCUUCAAGUGGUCAAGCG
CCAGAGCUGGuu

Npf1 Divalent Mv-RNA:

818/438,
(SEQ ID NO: ______)
UAGUGUGACAUGAACGACAGAUUUCUGUU
GUUCCAGUCGAUCUUCAAGUGAUCGACUGGGUUACAUUA
uu
617/587,
(SEQ ID NO: ______)
AUUUCAAUCAACCUAUUUAUUUaUGAAUA
AAUAGCUUCUAUUGUUCAAGUCAAUAGAAGAGAUUGAAAU
uu
711/591,
(SEQ ID NO: ______)
ACUAUUUUCAAUCAACCUAUUUaUGAAUAGGU
UGUGAAAACAAUUUCAAGUAUUGUUUUCAGAAGAUAGUuu
388/806,
(SEQ ID NO: ______)
GUUACAUUAUAGAAACAAUAAUAUUUGUAUUA
UUGUGUACCGGACUUCAAGUGUCCGGUACAUAGUGUAGU
uu
29/215,
(SEQ ID NO: ______)
AGCAGCUUGAGUGUAGUAGCGGUCUUGACCGC
UACUCCUACCAAGUUCAAGUCUUGGUAGGUCGAGUUGCU
uu
695/105,
(SEQ ID NO: ______)
GGCGAUGGCGACGGCGAUUCUUGAAUUGCCG
UUUCUGUGCUUUCAAGUAGCACAGAAAUGCUAUCGUUuu

NPF1 set linked as ssRNA:

(SEQ ID NO: ______)
UAGUGUGACAUGAACGACAGAUUUCUGUUGUUCCAGUCGA
UCUUCAAGUGAUCGACUGGGUUACAUUAuuAUUUCAAUCA
ACCUAUUUAUUUaUGAAUAAAUAGCUUCUAUUGUUCAAGU
CAAUAGAAGAGAUUGAAAUuuACUAUUUUCAAUCAACCUAU
UUaUGAAUAGGUUGUGAAAACAAUUUCAAGUAUUGUUUUC
AGAAGAUAGUuuGUUACAUUAUAGAAACAAUAAUAUUUGUA
UUAUUGUGUACCGGACUUCAAGUGUCCGGUACAUAGUGUA
GUuuAGCAGCUUGAGUGUAGUAGCGGUCUUGACCGCUACU
CCUACCAAGUUCAAGUCUUGGUAGGUCGAGUUGCUuuGGC
GAUGGCGACGGCGAUUCUUGAAUUGCCGUUUCUGUGCUU
UCAAGUAGCACAGAAAUGCUAUCGUUuu

Each set was then folded in co-fold to check for structural deviations as shown below:

Alter the Sequence Using this Invention

The resulting sequence design and secondary structure is in accordance with the guidelines provided by [Hauser, PCT/US2016/048492]. Using the designs and methods of the invention in this application, the sequence can be altered to form aptamer-driven surfaces by replacing many or all of the sequences containing various loops (BOLD) are replaced with an aptamer/intramer motif [FIG. 4a]. Alternatively, new aptamer/intramer structures can be added in-between [FIG. 4b] each MV-RNA link (UNDERLINED), as shown below.

Array of Linked Ecb Mv-RNA:

(SEQ ID NO: ______)
CGUUGGAAGGUGAAUUUGGUUCCAAAUUCAAAAGAACCCUUCAAG
UGGGUUCUUUUCUUUCAACGuaUUUUGGUGUUAAUUAUGAAAua
UUUCAUAAUUGAGAGAGGUUCAAGUCCUCUCUCAACAUCAAGCuu
ACGUUGUACAGAGUGCUUUUauAAAAGCACUCCGUCAAUAAUUCA
AGUUUAUUGACGUUGUACAGAGUuuAAUUAGCUGAUCCUUCGCU
GUUCAGCGAAGGAUUUACUCUAUUUCAAGUAUAGAGUAAUUAGCU
GAUUuuCAUUCGCGAACACCGCACUGGUUUACCAGUGCGGGACG
AUAAAUUCAAGUUUUAUCGUCAUUCGCGAAUGuuCUGGUUCUGCU
GCCAUAAUAUUUAUAUUAUGGCCGCUUGACCUUCAAGUGGUCAAG
CGCCAGAGCUGGuuUAGUGUGACAUGAACGACAGAUUUCUGUUG
UUCCAGUCGAUCUUCAAGUGAUCGACUGGGUUACAUUAuuAUUUC
AAUCAACCUAUUUAUUUaUGAAUAAAUAGCUUCUAUUGUUCAAGU
CAAUAGAAGAGAUUGAAAUuuACUAUUUUCAAUCAACCUAUUUaU
GAAUAGGUUGUGAAAACAAUUUCAAGUAUUGUUUUCAGAAGAUAG
UuuGUUACAUUAUAGAAACAAUAAUAUUUGUAUUAUUGUGUACC
GGACUUCAAGUGUCCGGUACAUAGUGUAGUuuAGCAGCUUGAGUG
UAGUAGCGGUCUUGACCGCUACUCCUACCAAGUUCAAGUCUUGG
UAGGUCGAGUUGCUuuGGCGAUGGCGACGGCGAUUCUUGAAUU
GCCGUUUCUGUGCUUUCAAGUAGCACAGAAAUGCUAUCGUUau

Design New, or Insert Pre-Existing Aptamer/Intramer Sequences into MV-RNA Loops

Using SELEX, one skilled in the art can design an aptamer/intramer sequence specific for surface moieties. However, this can be costly and time consuming. An interim design allowing rapid testing of numerous surface substance candidates using well-studied aptamers with suitable pK from the public domain is provided [FIG. 12]. Aptamer sequences and surface substance sequences are not the subject of this invention, but this invention provides a useful tool making programmable nanoparticle surfaces whether derived from known or novel aptamer/moiety relationships. The TAT aptamer was chosen as a suitable aptamer with an adequate disassociation constant (<600 nM) to drive surface assembly, then adapted using the methods and designs of this invention. The TAT aptamer sequence is shown below:

(SEQ ID NO: ______)
HIV-1 Tat, CCAGAUCUGAGCCUGGGAGCUCUCUGG

The example HIV-1 TAT loop sequence was used to replace many of the MV-RNA loops in order to present the surface forming aptamer/intramers onto the surface of the nanoparticle core. As shown in the exemplar sequence below, twelve (12) MV'S with 7NT loops were replaced with the TAT aptamer sequence (aptamer underlined).

(SEQ ID NO: ______)
GUUGGAAGGUGAAUUUGGUUCCAAAUUCAAAAGAACCCGAUCUGA
GCCUGGGAGCUCUCGGGUUCUUUUCUUUCAACGuaUUUUGGUGUUA
AUUAUGAAAuaUUUCAUAAUUGAGAGAGGGAUCUGAGCCUGGGAGCU
CUCCCUCUCUCAACAUCAAGCuuACGUUGUACAGAGUGCUUUUau
AAAAGCACUCCGUCAAUAAGAUCUGAGCCUGGGAGCUCUCUUAUUGA
CGUUGUACAGAGUuuAAUUAGCUGAUCCUUCGCUGUUCAGCGAA
GGAUUUACUCUAUGAUCUGAGCCUGGGAGCUCUCAUAGAGUAAUUAG
CUGAUUuuCAUUCGCGAACACCGCACUGGUUUACCAGUGCGGG
ACGAUAAAGAUCUGAGCCUGGGAGCUCUCUUUAUCGUCAUUCGCGAA
UGuuCUGGUUCUGCUGCCAUAAUAUUUAUAUUAUGGCCGCUUGA
CCGAUCUGAGCCUGGGAGCUCUCGGUCAAGCGCCAGAGCUGGuuUAG
UGUGACAUGAACGACAGAUUUCUGUUGUUCCAGUCGAUCGAUCU
GAGCCUGGGAGCUCUCGAUCGACUGGGUUACAUUAuuAUUUCAAUCA
ACCUAUUUAUUUaUGAAUAAAUAGCUUCUAUUGGAUCUGAGCCUGG
GAGCUCUCCAAUAGAAGAGAUUGAAAUuuACUAUUUUCAAUCAACC
UAUUUaUGAAUAGGUUGUGAAAACAAUGAUCUGAGCCUGGGAGCUCU
CAUUGUUUUCAGAAGAUAGUuuGUUACAUUAUAGAAACAAUAAUA
UUUGUAUUAUUGUGUACCGGACGAUCUGAGCCUGGGAGCUCUCGUCC
GGUACAUAGUGUAGUuuAGCAGCUUGAGUGUAGUAGCGGUCUU
GACCGCUACUCCUACCAAGGAUCUGAGCCUGGGAGCUCUCCUUGGUA
GGUCGAGUUGCUuuGGCGAUGGCGACGGCGAUUCUUGAAUUGC
CGUUUCUGUGCUGAUCUGAGCUGGGAGCUCUCAGCACAGAAAUGCU
AUCGUU

A 12-unit nanoparticle containing surface aptamers (ECB-3) and a 12-unit nanoparticle without aptamers (ECB-2) was created using the sequence above. To complete the nanoparticle sequence design, a stem was placed before and after the candidate sequences to stably connect the 5′ to the 3′ ends. On the 5′ side, a T7 transcription motif was added and Rho termination sequence was added to 3′ end in support of in-vitro or intracellular production in e. coli. This promoter and terminator for the transcript can be exchanged production and cell types (i.e., ubiquitin promoter/PIN2 terminator). A streptavidin aptamer was added 3′ of the transcript to enable affinity pull-down assays. This example sequence was further altered to support cloning strategies into pBluescript KS+. The full sequences are shown below:

>ECB-2; 12 MV'S, NO TAT APTAMER 942 nt
(SEQ ID NO: ______)
CGCGCGTAATACGACTCACTATAGGGGCCTAGGGGCAACGCCGGTCCGU
UGGAAGGUGAAUUUGGUUCCAAAUUCAAAAGAACCCUUCAAGUGG
GUUCUUUUCUUUCAACGuaUUUUGGUGUUAAUUAUGAAAuaUUU
CAUAAUUGAGAGAGGUUCAAGUCCUCUCUCAACAUCAAGCuuACG
UUGUACAGAGUGCUUUUauAAAAGCACUCCGUCAAUAAUUCAAGU
UUAUUGACGUUGUACAGAGUuuAAUUAGCUGAUCCUUCGCUGUU
CAGCGAAGGAUUUACUCUAUUUCAAGUAUAGAGUAAUUAGCUGAU
UuuCAUUCGCGAACACCGCACUGGUUUACCAGUGCGGGACGAUA
AAUUCAAGUUUUAUCGUCAUUCGCGAAUGuuCUGGUUCUGCUGCC
AUAAUAUUUAUAUUAUGGCCGCUUGACCUUCAAGUGGUCAAGCGC
CAGAGCUGGuuUAGUGUGACAUGAACGACAGAUUUCUGUUGUU
CCAGUCGAUCUUCAAGUGAUCGACUGGGUUACAUUAuuAUUUCAA
UCAACCUAUUUAUUUaUGAAUAAAUAGCUUCUAUUGUUCAAGUCA
AUAGAAGAGAUUGAAAUuuACUAUUUUCAAUCAACCUAUUUaUGA
AUAGGUUGUGAAAACAAUUUCAAGUAUUGUUUUCAGAAGAUAGUu
uGUUACAUUAUAGAAACAAUAAUAUUUGUAUUAUUGUGUACCGG
ACUUCAAGUGUCCGGUACAUAGUGUAGUuuAGCAGCUUGAGUGUA
GUAGCGGUCUUGACCGCUACUCCUACCAAGUUCAAGUCUUGGUA
GGUCGAGUUGCUuuGGCGAUGGCGACGGCGAUUCUUGAAUUGC
CGUUUCUGUGCUUUCAAGUAGCACAGAAAUGCUAUCGUUauGACC
GGCGTTGCtTAGGCCGGTACCGCACCGACCAGAATCATGCAAGTGC
GTAAGATAGTCGCGGGCCGGGGCGTCGACAAGCGCCGacaacCGGCG
CTTTTTTtG (SEQ IQ NO: ______; structure shown
below)

>ECB-3; 12 MV'S, WITH TAT APTAMER 1111 nt
CGCGCGTAATACGACTCACTATAGGGGCCTAGGGCAACGCCGGTCCGU
UGGAAGGUGAAUUUGGUUCCAAAUUCAAAAGAACCCGAUCUGAGC
CUGGGAGCUCUCGGGUUCUUUUCUUUCAACGuaUUUUGGUGUUAA
UUAUGAAAuaUUUCAUAAUUGAGAGAGGGAUCUGAGCCUGGGAGCUC
UCCCUCUCUCAACAUCAAGCuuACGUUGUACAGAGUGCUUUUauA
AAAGCACUCCGUCAAUAAGAUCUGAGCCUGGGAGCUCUCUUAUUGAC
GUUGUACAGAGUuuAAUUAGCUGAUCCUUCGCUGUUCAGCGAAG
GAUUUACUCUAUGAUCUGAGCCUGGGAGCUCUCAUAGAGUAAUUAGC
UGAUUuuCAUUCGCGAACACCGCACUGGUUUACCAGUGCGGGA
CGAUAAAGAUCUGAGCCUGGGAGCUCUCUUUAUCGUCAUUCGCGAAU
GuuCUGGUUCUGCUGCCAUAAUAUUUAUAUUAUGGCCGCUUGAC
CGAUCUGAGCCUGGGAGCUCUCGGUCAAGCGCCAGAGCUGGuuUAGU
GUGACAUGAACGACAGAUUUCUGUUGUUCCAGUCGAUCGAUCUG
AGCCUGGGAGCUCUCGAUCGACUGGGUUACAUUAuuAUUUCAAUCAA
CCUAUUUAUUUaUGAAUAAAUAGCUUCUAUUGGAUCUGAGCCUGGGA
GCUCUCCAAUAGAAGAGAUUGAAAUuuACUAUUUUCAAUCAACCUA
UUUaUGAAUAGGUUGUGAAAACAAUGAUCUGAGCCUGGGAGCUCUCA
UUGUUUUCAGAAGAUAGUuuGUUACAUUAUAGAAACAAUAAUAU
UUGUAUUAUUGUGUACCGGACGAUCUGAGCCUGGGAGCUCUCGUCCG
GUACAUAGUGUAGUuuAGCAGCUUGAGUGUAGUAGCGGUCUUG
ACCGCUACUCCUACCAAGGAUCUGAGCCUGGGAGCUCUCCUUGGUAG
GUCGAGUUGCUuuGGCGAUGGCGACGGCGAUUCUUGAAUUGCC
GUUUCUGUGCUGAUCUGAGCCUGGGAGCUCUCAGCACAGAAAUGCUA
UCGUUauGACCGGCGTTGCtTAGGCCGGTACCGCACCGACCAGAAT
CATGCAAGTGCGTAAGATAGTCGCGGGCCGGGGCGTCGACAAGCGC
CGacaacCGGCGCTTTTTTtG (SEQ ID NO: ______;
structure shown below)

Design new, or insert pre-existing aptamer/intramer sequences in between MV-RNA loops

In support of the aptamer plurality used in this invention, a set of alternate single TAT-stems loops [FIG. 4b] of appropriate length are designed to reduce potential secondary structures deviations caused by sequence replication. Single TAT-stems are shown below.

Single-TAT Aptamer Stem Set 1:

AGCCGGGCAGCUCCGACCAGAUCUGAGCCUGGGAGCUCUCUGGUCGGA
GCUGCCCGGCUuu (Single-TAT Aptamer Stem 1a; SEQ ID
NO: ______)
CAGGGAGCUGCGGCGUCCAGAUCUGAGCCUGGGAGCUCUCUGGACGCC
GCAGCUCCCUGuu (Single-TAT Aptamer Stem 1b; SEQ ID
NO: ______)
GACCCCGCCGUAGCCACCAGAUCUGAGCCUGGGAGCUCUCUGGUGGCU
ACGGCGGGGUCuu (Single-TAT Aptamer Stem 1c; SEQ ID
NO: ______)

Single-TAT Aptamer Stem Set 2:

CGACGGGCAGCUCGGACCAGAUCUGAGCCUGGGAGCUCUCUGGUCCGA
GCUGCCCGUCGuu (Single-TAT Aptamer Stem 2a; SEQ ID
NO: ______)
GACGGAUCUGCGGCGUCCAGAUCUGAGCCUGGGAGCUCUCUGGACGCC
GCAGAUCCGUCuu (Single-TAT Aptamer Stem 2b; SEQ ID
NO: ______)
GCCAUUGCCGUAGCCACCAGAUCUGAGCCUGGGAGCUCUCUGGUGGCU
ACGGCAAUGGCuu (Single-TAT Aptamer Stem 2c; SEQ ID
NO: ______)

The structure of an exemplary single-TAT Aptamer Stem is shown below:

Additionally, a set of dual-TAT 3-way junctions were also designed with variable and conserved (RED, ORANGE) regions. The dual TAT aptamer supports the nanoparticle core/surface stem ratio, binding plurality, and disassociation constant expectations. Dual-TAT 3-way junctions are shown below.

Dual-TAT Aptamer Stem Set 1:

GGGCAGCUCCGAAACCAGAUCUGAGCCUGGGAGCUCUCUGGaCGCCAUCU
GAGCCUGGGAGCUCUGGCGAAUCGGAGCUGCCCuu (Dual-TAT
Aptamer Stem 1a; SEQ ID NO: ______)
GAGCUGCGGCGUAACCAGAUCUGAGCCUGGGAGCUCUCUGGaCGCCAUCU
GAGCCUGGGAGCUCUGGCGAAACGCCGCAGCUCuu (Dual-TAT
Aptamer Stem 1b; SEQ ID NO: ______)
CCGCCGUAGCCAAACCAGAUCUGAGCCUGGGAGCUCUCUGGaCGCCAUCU
GAGCCUGGGAGCUCUGGCGAAUGGCUACGGCGGuu (Dual-TAT
Aptamer Stem 1c; SEQ ID NO: ______)

Dual-TAT Aptamer Stem Set 2 (with different stem composition)

GGGCAGCUCGGAAACCAGAUCUGAGCCUGGGAGCUCUCUGGaCGCCAUCU
GAGCCUGGGAGCUCUGGCGAAUCCGAGCUGCCCuu (Dual-TAT
Aptamer Stem 2a; SEQ ID NO: ______)
GAUCUGCGGCGUAACCAGAUCUGAGCCUGGGAGCUCUCUGGaCGCCAUCU
GAGCCUGGGAGCUCUGGCGAAACGCCGCAGAUCuu (Dual-TAT
Aptamer Stem 2b; SEQ ID NO: ______)
UUGCCGUAGCCAAACCAGAUCUGAGCCUGGGAGCUCUCUGGaCGCCAUCU
GAGCCUGGGAGCUCUGGCGAAUGGCUACGGCAAuu (Dual-TAT
Aptamer Stem 2c; SEQ ID NO: ______)

The structure of an exemplary dual-TAT Aptamer Stem is shown below:

>ECB-1; 12 MV'S REPEATED TWICE, WITH DUAL APTAMERS
PLACED EVERY 3RD MV
(SEQ ID NO: ______)
CGCGCGTAATACGACTCACATATAGGGGCCTAGGGCAACGCCGGTCCGT
TGGAAGGTGAATTTGGTTCCAAATTCAAAAGAACCCTTCAAGTGG
GTTCTTTTCTTTCAACGTaTTTTGGTGTTAATTATGAAATaTTTCATA
ATTGAGAGAGGTTCAAGTCCTCTCTCAACATCAAGCTTACGTTGTA
CAGAGTGCTTTTaTAAAAGCACTCCGTCAATAATTCAAGTTTATTGA
CGTTGTACAGAGTTTGGGCAGCTCCGAAACCAGATCTGAGCCTGGGAG
CTCTCTGGaCGCCATCTGAGCCTGGGAGCTCTGGCGAATCGGAGCTG
CCCTTAATTAGCTGATCCTTCGCTGTTCAGCGAAGGATTTACTCTAT
TTCAAGTATAGAGTAATTAGCTGATTTTCATTCGCGAACACCGCAC
TGGTTTACCAGTGCGGGACGATAAATTCAAGTTTTATCGTCATTCG
CGAATGTTCTGGTTCTGCTGCCATAATATTTATATTATGGCCGCTT
GACCTTCAAGTGGTCAAGCGCCAGAGCTGGTTGAGCTGCGGCGTAAC
CAGATCTGAGCCTGGGAGCTCTCTGGACGCCATCTGAGCCTGGGAG
CTCTGGCGAAACGCCGCAGCTCTTTAGTGTGACATGAACGACAGATT
TCTGTTGTTCCAGTCGATCTTCAAGTGATCGACTGGGTTACATTAT
TATTTCAATCAACCTATTTATTTaTGAATAAATAGCTTCTATTGTTCA
AGTCAATAGAAGAGATTGAAATTTACTATTTTCAATCAACCTATTTa
TGAATAGGTTGTGAAAACAATTTCAAGTATTGTTTTCAGAAGATAG
TTTCCGCCGTAGCCAAACCAGATCTGAGCCTGGGAGCTCTCTGGaCGC
CATCTGAGCCTGGGAGCTCTGGCGAATGGCTACGGCGGTTGTTACATT
ATAGAAACAATAATATTTGTATTATTGTGTACCGGACTTCAAGTGTC
CGGTACATAGTGTAGTTTAGCAGCTTGAGTGTAGTAGCGGTCTTG
ACCGCTACTCCTACCAAGTTCAAGTCTTGGTAGGTCGAGTTGCTTT
GGCGATGGCGACGGCGATTCTTGAATTGCCGTTTCTGTGCTTTCA
AGTAGCACAGAAATGCTATCGTTTTGGGCAGCTCGGAAACCAGATCT
GAGCCTGGGAGCTCTCTGGaCGCCATCTGAGCCTGGGAGCTCTGGC
GAATCCGAGCTGCCCTTCGTTGGAAGGTGAATTTGGTTCCAAATTCAA
AAGAACCCTTCAAGTGGGTTCTTTTCTTTCAACGTaTTTTGGTGTTA
ATTATGAAATaTTTCATAATTGAGAGAGGTTCAAGTCCTCTCTCAAC
ATCAAGCTTACGTTGTACAGAGTGCTTTTaTAAAAGCACTCCGTCA
ATAATTCAAGTTTATTGACGTTGTACAGAGTTTGATCTGCGGCGTAAC
CAGATCTGAGCCTGGGAGCTCTCTGGaCGCCATCTGAGCCTGGGAG
CTCTGGCGAAACGCCGCAGATCTTAATTAGCTGATCCTTCGCTGTTCA
GCGAAGGATTTACTCTATTTCAAGTATAGAGTAATTAGCTGATTTT
CATTCGCGAACACCGCACTGGTTTACCAGTGCGGGACGATAAATT
CAAGTTTTATCGTCATTCGCGAATGTTCTGGTTCTGCTGCCATAAT
ATTTATATTATGGCCGCTTGACCTTCAAGTGGTCAAGCGCCAGAG
CTGGTTTTGCCGTAGCCAAACCAGATCTGAGCCTGGGAGCTCTCTGGa
CGCCATCTGAGCCTGGGAGCTCTGGCGAATGGCTACGGCAATTTAGT
GTGACATGAACGACAGATTTCTGTTGTTCCAGTCGATCTTCAAGTG
ATCGACTGGGTTACATTATTATTTCAATCAACCTATTTATTTaTGAA
TAAATAGCTTCTATTGTTCAAGTCAATAGAAGAGATTGAAATTTACT
ATTTTCAATCAACCTATTTaTGAATAGGTTGTGAAAACAATTTCAAG
TATTGTTTTCAGAAGATAGTTTCCGCCGTAGCCAAACCAGATCTGAGC
CTGGGAGCTCTCTGGaCGCCATCTGAGCCTGGGAGCTCTGGCGAATG
GCTACGGCGGTTGTTACATTATAGAAACAATAATATTTGTATTATTGT
GTACCGGACTTCAAGTGTCCGGTACATAGTGTAGTTTAGCAGCTT
GAGTGTAGTAGCGGTCTTGACCGCTACTCCTACCAAGTTCAAGTC
TTGGTAGGTCGAGTTGCTTTGGCGATGGCGACGGCGATTCTTGAA
TTGCCGTTTCTGTGCTTTCAAGTAGCACAGAAATGCTATCGTTTTG
ACCGGCGTTGCCtTAGGCCGGTACCGCACCGACCAGAATCATGCAAG
TGCGTAAGATAGTCGCGGGCCGGGGCGTCGACAAGCGCCGacaacCG
GCGCTTTTTTtG

### Example 2: Aptamer-Driven Multi-Part Nanoparticle Surfaces Targeting

Spodoptera frugiperda (Fall armyworm)

As described herein, the invention can be applied to a polynucleotide nanoparticle-contributing to multi-part surfaces with programmable composition that provide modes of action, cellular recognition and/or uptake in a highly specific manner. For bio-medical and agricultural uses, this is a useful approach to improve upon the bioavailability and function of polynucleotide nanoparticles.

This example describes the assembly of a multi-part aptamer-driven nanoparticle surface sequence according to the invention. The programmable surface composition is enabled by a plurality of intramer/aptamer sequences specifically designed to recruit targeted moieties onto restricted regions of the nanoparticle surface. The single-stranded self-forming polynucleotide nanoparticle core delivers an array of active RNAi triggers targeting genes in Fall armyworm. With the addition of this invention, the core polynucleotide sequence is supplemented with a plurality of aptamer/intramer sequences which create a controlled geography surface moieties. The formed surface presents characteristics and/or function atypical of polynucleotides.

This non-limiting example illustrates the benefit of binding groups of peptides onto the nanoparticle surface as a means to control cell penetration, and protecting moieties whether product in-vitro or intracellularily.

Preparation of a Polynucleotide Nanoparticle Sequence

In this nanoparticle core example, three genes are targeted: (1) vATPase-A, (2) COPI beta prime, and (3) COPI sub beta prime. Multiple nanoparticle core sequences were assembled with various RNAi trigger pluralities according to [Hauser, PCT/US2016/048492]. The FAW target genes are: (with BOLD regions published by Monsanto patent application US20170183683A1) are shown below.

Spodoptera frugiperda 1 v-ATPase A

(SEQ ID NO: ______)
GGTGACATGGCCACCATCCAGGTATACGAAGAAACATCAGGTGTA
ACTGTAGGTGACCCCGTGCTGCGTACCGGCAAGCCCCTGTCCGT
AGAGCTGGGTCCTGGTATCCTCGGCTCCATCTTTGACGGTATCCA
GCGGCCACTGAAGGACATCAACGAGCTCACACAGTCCATCTACAT
CCCCAAGGGTGTCAACGTACCCTGCCTTGGACGTGATGTCACCTG
GGAATTCAACCCCTTGAATGTTAAGGTCGGCTCCCACATCACCGG
AGGAGACTTGTACGGTATCGTACACGAGAACACATTGGTTAAGC
ATAAGATGTTGATCCCACCCAAGGCCAAGGGTACCGTCACCTAC
ATCGCGCCCTCCGGCAACTACAAAGTCACTGACGTAGTGTTGGA
GACGGAGTTCGACGGCGAGAAGGAGAAGTACACCATGTTGCAA
GTATGGCCGGTGCGCCAGCCCCGCCCCGTCACTGAGAAGCTGC
CCGCCAACCACCCCCTGCTCACCGGACAGAGAGTGCTCGACTCT
CTCTTCCCTTGTGTCCAGGGTGGTACCACGGCCATCCCCGGCGC
CTTCGGTTGTGGCAAGACTGTCGTCTCACAGGCTCTGTCCAAGTA
CTCCAACTCTGACGTCATCATCTACGTCGGATGCGGTGAACGTGG
TAACGAGATGTCTGAGGTACTGCGTGACCTCCCCGAGCTGACGG
TGGAGATCGAGGGCATGACCGAGTCCATCATGAAGCGTACCGCG
CTCGTCGCCAACACCTCCAACATGCCTGTAGCCGCCCGAGAGGC
TTCCATCTACACCGGTATCACCCTCTCCGAGTACTTCCGTGATATG
GGTTACAACGTGTCCATGATGGCTGACTCCACCTCTCGTTGGGCC
GAAGCTCTTCGTGAGATCTCAGGTCGTCTGGCTGAGATGCCTGCC
GACTCCGGTTACCCCGCCTACCTGGGAGCCCGTCTGGCCTCCTT
CTACGAGCGTGCCGGACGTGTGAAGTGCCTGGGTAACCCCGACA
GGGAGGGCTCCGTGTCCATCGTGGGCGCCGTGTCGCCGCCCGG
AGGTGACTTCTCCGACCCCGTGACGGCCGCCACGCTGGGTATCG
TGCAGGTGTTCTGGGGT

Spodoptera frugiperda 2 COPI Coatomer beta subunit

(SEQ ID NO: ______)
AACTATGAAACACATTCGCAGATCAAGAGATTCGAGGTATGCGAC
CTACCGGTGCGCGCCGCTAAGTTCGTGCCTCGCAAGAACTGGGT
GATTACTGGCTCCGATGACATGCAGATTCGCGTGTTCAACTACAA
CACGCTTGAGAGAGTGCACGCCTTTGAGGCGCATTCTGACTACGT
CAGGTGCATCGCGATACATCCCACACAGCCATACATCCTCACCAG
CAGCGATGACCTATTGATCAAGCTGTGGAACTGGGAACGCAACTG
GGCATGCCAGCAAGTGTTCGAGGGCCACACACATTATGTGATGCA
GATCGTCATCAACCCTAAAGACAACAACACATTCGCTAGTGCTAGT
CTCGACACCACCGTCAAAGTATGGCAGCTTGGCTCTTCAATTTCC
AACTTCACATTAGAAGGCCACGAGAAAGGCGTGAACTGCGTCGAC
TACTACCACGGCGGCGACAAGCCTTACCTCATAAGTGGTGCCGAC
GATCGCCTCGTCAAAATATGGGACTACCAGAATAAAACATGTGTC
CAGACATTGGAGAGTCACACGCAGAATGTGACAGCCGTGTCGTTC
CACCCGGAGCTGCCGATCCTGATGACTGGCTCAGAGGACGGCAC
CATCAGAATATGGCACGCAGGCACTTACAGACTTGAATCCTCGCT
CAACTATGGCTTCGAGAGAGTCTGGACCATTTCCTCCATGCACGG
CTCTAACAACGTAGCTATTGGTTACGACGAGGGCACGATCATGAT
CAAGGTGGGCCGCGAAGAGCCGGCCATTTCCATGGACGTCAATG
GAGGCAAGATTATTTGGGCGAAGCATTCTGAGATGCAGCAAGTCA
ACCTGAAGGCGTTGCCAGAAGGCACAGAGATAAAAGATGGAGAG
CGGGTTCCAGTTATGGCGAAGGACATGGGATCCTGTGAGATTTAT
CCACAAACTATAGCACACAACCCGAACGGTCGGTTCGTGGTAGTG
TGCGGGGACGGCGAGTACATTATTTACACGGCCATGGCACTTAG
GAACAAGGCGTTCGGCACTGCACAGGAGTTCGTCTGGGCGTTTG
ATAGCTCAGAGTATGCGACACTTGAGAACTCCAGCACCATCAAGG
TGTTCAAGAACTTCAAAGAGAGAAAGAGTTTCAAGCCTGAGTATG
GTGCTGAAGGAATATACGGCGGTTTCATGTTGGGCGTGAAGTCCA
TCAGCGGTGTGGCGTTCTCCTTCTATGATTGGGAGAACTTGGAGT
TGATCAGACGGATTGAGATCCAGCCGCGGCACGTGTACTGGTCG
GAGAGCGGCAACCTGGTGTGCCTGGCGGCTGATGACTCGTACT
ACGTGCTCAAGTATAATGCAGCTGTTGTGACGCGAGCTCGCGAA
ACCAACTCCAACATCACAGAAGACGGCA1CGAAGACGCTTTTGA
GGTCGTGGGTGCAGTGAACGAGGTGGTAAAGACAGGACTATGG
GTGGGCGACTGCTTCATCTACACGAATTCCTTGAACAGAATAAA
CTACTACGTCGGCGGAGAGATCGTCACCATATCCCACCTGGACCA
CACCATGTACATCCTCGGATACGTCGCTAAGGAGAACAGACTGTA
CCTAAATGACAAGGAGTTGAACATAGTGTCGTACTCGCTGCTGCT
GTCGGTACTGGAGTACCAGACGGCGGTGATGCGCGCAGACTTCG
AGACAGCAGACCGGGTGCTCCCCACCATACCGCAAGAGCATCGC
ACCAGAGTCGCTCACTTCCTAGAGAAACAAGGCTTCAAACAACAA
GCTCTAGCAGTCTCTACGGAGCCTGAACACCAGTTCGAGCTGGCT
CTCTCACTGGGAGAACTTAGAAGAGCAAAGCAGTTGGCCGAGGA
GGCGGCATTGGCCGAGGGTTCCACCTCCCGATCCTCAGCGGCAC
GGTGGTCTCGGCTGGGAGCGGCCGCCGCCGCCGCTGCAGACAC
GGAACTCACCAAGGCCTGCTATCAGAGCGCTAAGGACTACAGTG
CCCTCCTTCTATTCGCAGCTAGTACTGGTGACAAAGAACTGTTGAA
GAGTGTAGCACAGATGTCCTCGGAGGAAAACGCGGAGAATATCTC
ATTCGTGGCCTACTTTATGCTGAATGATCTCGAATCCTGTCTGAAG
CTGCTCATACAGCGGGACAAGCTGCCTGAAGCCGCCTTCTTTGCC
AGATCATACATTCCATCGAAAATGTCGGAAGTGGTGAAGTTATGG
CGCGAGTCCACCAGCGCCACCAACACGAAGCTCGGCCAGTCGCT
CGCCGACCCGGAGCAGTATGAGAATTTGTTCCCCGAGTTTGCGCA
ATCATTGGAGTTGGAGAGATTTCAGCGCGAGTATGGGTACGAGCA
GAGTTGCTTGCTAGCGAACTTGCCGGTCGACTCGAAGACTTGCAA
CCTTGAGCGTAACCTTGCCACCGAGAAGGAGGAGGCCGAGCAAC
GCGGCTTTAAGCTGAGCTCCGCTGCAGTGGCGCGGTCTCCTGCA
CACAGCTCAAATGATCTTGGTCCTGACGACCGAGTCACAAACAAT
GATAGTCCTTCCCACCCGGTGCCGGAGAGCACGGTGGCGACCGC
GGCGCAGCAGGATCTGAAGAAACGGCGCGACTCTCTCGACATCA
TGGAGGAACTGGAGCGCGAGATCGAAGACATCGTCCTGGACGGC
ACGGTCGAGAGCGTGGATCTGTCGGACGATGTAGATTTCATGGAC
TAA

Spodoptera frugiperda 3 COPI coatomer beta prime subunit

(SEQ ID NO: ______)
ATGTGTATAAGAGACAGCTATACTCTGATCAACTTCCCGACTGATT
CAGAGCCTTACAATGAGATGCAGCTCAAGCTGGATCTTGAGAAAG
GYGACACAAAGAAGAAAATAGAAGCATTAAAGAAGGTAATAGGTA
TYATCYTKTCTGGKGAGAAGATWCCYGGTCTATTGATGATCATCAT
CCGATTYGTGCTGCCCCTGCAAGACCACACCATCAAGAAGCTGTT
GCTKATCTTCTGGGAAATAGTGCCAAAGACCACTCCTGATGGCAA
GCTCATGCAGGAGATGATCCTTGTCTGTGATGCTTACAGAAAGGA
TCTGCAACACCCCAATGAGTTCATCCGTGGCTCCACCCTGCGCTT
CCTCTGTAAACTGAAGGAGCCTGAACTCCTCGAGCCTTTGATGCC
CGCCATCAGAGCTTGTCTTGACCACCGCCACTCATATGTCAGGAG
GAATGCTGTTCTTGCCATATTTACTATCTACAGGAACTTTGAATTC
CTGATCCCTGATGCTCCAGAGCTAGTAGCAAACTTCTTGGAGACA
GAGCAGGACATGTCCTGCAAGAGGAATGCATTTCTTATGCTGCTC
CATGCTGACCAGGAGCGGGCACTGTCCTACCTCTCATCCAGGCT
GGATAATGTACAGGGCTTTGGAGATATCCTTCAGTTGGTTATTGTA
GAACTTATTTACAAGGTTTGCCACGCGAATCCATCAGAGAGGTCT
CGTTTCATCCGTACAGTATACGGCTTACTGAACGCGACCAGTGCC
GCCGTGCGGTACGAGGCTGCCGGTACTCTCGTTACACTGTCTAAC
GCGCCTGCTGCTATCAAGGCGGCGGCAGCATGCTACATCGACCT
AATAGTGAAGGAGAGCGACAACAACGTGAAGCTGATAGTGGTGG
GTCGGCTGGGAGCGCTGCGCGCGGAGGCGGGCGAGGCGGCGG
CGCGCGCGCTGCCCGAGCTGGCCATGGACGTGCTGCGCGTGCT
GGCCGCCTCCGACCTGGACGTGCGCCGACACACGCTGGCGCTG
GCGCTCGACCTCGTGTCCTCCCGCCACGCCGAGGACCTCGTGCA
GGTGCTGCGGAAGGAGGCCGCCCGGGCTACCAACGCAGACCAC
GATGATGCTGCCAAGTACAGGCAGCTACTTGTTAGAGCTCTGCAC
CGAGCTGCACTCAAGTTCCCCGAAGTAGCCGGCAGTGTAGCCCC
GGCGCTGCTGGAGTTGCTGGGCGACGGCAGTGAGCCGGCCGCG
CAGGATGTCATGCTGTTCCTACGGTCCGCTCTACATACCTTCGTG
ACCTACGGATCATATCTACCAGAAACTGTTGGAGGCGGTGCCCG
GTATCAAAGTGGTAAGATAGCGCGGTCTGCGCTGTGGCTGCTGG
CCCAGTTCGCTGAGACTCCGGAACGCGCCAAGGATGCCTTGGAT
GTACTCGCCAACGTCATACCTTCCCTTAGCGGACAAGAGGATAA
GGAAGAATCCGAGTCGGCAGCTAAGGCCCAGGACACTTCAGCT
CCACGACAGCTTGTCACCAGTGATGGAACTTATGCTTCGCAGTC
TGCTTTTAACTTGCCAGTTAGCCAAGCGGCTCCAACCCACGCGG
GTCTATGGGCGGCACTAGGCGAGGGCGAGAGCTTCACGGCGGC
GTGCGCGTGCTCGGCGCTGTGCAAGCTGGCGCTGAAGCTGTCGG
GCCGGGCCGCGACCGCCGCGCTGCAGCTGGCCGCGCGCCTGCT
GGCCGCCCACAAGCTGGCCGCCGGCCTCACCGCCGACGACGCC
GAGCACGGCGCGCGATGTATACTGGCCGCGAGACACCGCCCGC
CCGTCGTACAGGAAGCGCTGCTGCAACGCTCCTCTGCTGCGCTC
GCCGCGCTGCTAGCGCTGCCTGACCGAGCTACTAATCTGCTTGAT
GATGCTGATAAGGAACGTGAGCCAAAGAAGCAAGACAACAAGGT
GGAGGTGGAACAAGGCATCGTGTTCGCCCAGTTGGCTGGGAACT
CCGCCGCCTCCACACACCACGACATGTTCGAGCTATCGCTCACTA
AGGCACTGCAAGGCCGCAGTACCGGCGTGAGCGAGGAGCGCGG
CAAGCTGTGGAAGGTGACGCAGCTGACCGGGTTCTCCGACCCCG
TGTACGCCGAGGCCATCGTCGCCGTCAACCAGTACGACATCGTG
CTCGACGTACTCGTCGTCAACCAGACCGACGACACGCTGCAGAA
CTGCTGCGTGGAGCTGGCGACGCTGGGCGACCTGCGGCTGGTG
GAGCGGCCCGGGGCCGTGGTGCTGGCCCCCAGGGACTACGCCA
CCATCAAGGCGCACGTCAAGGTCGCCTCCACCGAGAACGGCATC
ATCTTCGGGAACATTGTGTACGAGGTATCGGGCGCGTCGATGGA
CCGCGGCGTGGTGGTCCTGAACGACATCCACATCGACATCGTGG
ACTACATCCAGCCCGCCGTCTGCACCGACGCAGACTTCAGGCAG
ATGTGGGCCGAGTTCGAGTGGGAGAATAAGGTGTCCGTGAACAC
GAACATCACGGACCTGCGCGAGTACTTACAACATTTACTAGCTTC
CACAAACATGAAGTGTTTGACGCCTGAAAAGGCATTATCGGGTCA
ATGCGGGTTCATGGCAGCCAACCTGTACGCGCGGTCCATATTCG
GCGAGGACGCGCTGGCCAACCTGAGCATCGAGACCCCGCTGCAC
AAACCCAACTCGCCCGTCGTCGGACATGTCAGGATCAGGGCCAA
GAGCCAGGGTATGGCGCTGTCTCTAGGCGACAAAATCAACATGAT
GCACAAGACGCCGCAACAGAAGACCCCCTCCAACCCCATCCCCG
CCGCGTAA

The individual MV-RNA designs are derived from the software application ‘RNAi Cloud’ projects #P01034, entitled ‘FAW COPIbp/vATPa/COPIb’, project #P01033, entitled ‘FAW COPI beta prime’, project #P01032, entitled ‘FAW vATPase-A’, and project #P01031, entitled ‘FAW COPI Sub beta’.

A few of the MV-RNA designs were selected based on the confidence ranking in the RNAi Cloud software. Each set of MV-RNA were then grouped and linked as instructed. The results are as follows

For vATPase Targeting:

Divalent vATPa_545/303
(SEQ ID NO:)
GGUAUCGUACUCCCUUGUGUUUCAAGUACACAAGGGAAGAGAGAGUC
GAUUCAAGUUGUGUUCUCGUGUACGAUACCuu
Divalent vATPa_542/388
(SEQ ID NO:)
CGGCAACUACUCUUCCCUUGUUCAAGUCAAGGGAAGAGAGAGUCGAG
GUUCAAGUCAGUGACUUUGUAGUUGCCGuu
Divalent vATPa_320/501
(SEQ ID NO.)
AAGCUGCCCGAGCAUAAGAUUUCAAGUAUCUUAUGCUUAACCAAUCU
UUCAAGUGGGGUGGUUGGCGGGCAGCUUuu

For COPI SUB-BETA targeting:

Trivalent COPI_sub_beta_521/731/216,
(SEQ ID NO: ______)
AGUCCCAUAUUUUGACGAGGUUUCAAGUACGUUGUUAGAGCCGUGCA
UCUUCAAGUGAUGUAUGGCUGUGUGGGAUUuu
Divalent COPI_SB_1419/1240)
(SEQ ID NO: ______)
UGGCGUUCUCCGGCAUCGAAcUCAAGUUUCGAUGCCGUCUUCUGUGA
UUUCAAGUAUCAUAGAAGGAGAACGCCAuu
Divalent COPI_SB_1241/1108
(SEQ ID NO: ______)
UGCGACACUUUCUAUGAUUUUCAAGUAAUCAUAGAAGGAGAACGCCA
UUCAAGUUGGAGUUCUCAAGUGUCGCAuu

For COPI BETA PRIME targeting:

Divalent COPI_BP_1525/1323
(SEQ ID NO: ______)
UCAUAUCUACCGACAGCUUGUUCAAGUCAAGCUGUCGUGGAGCUGAU
GUUCAAGUCAACAGUUUCUGGUAGAUAUGAuu
Divalent COPI_BP_1532/1446
(SEQ ID NO: ______)
UGUAUUCGCCUUGUCACCAUUCAAGUUGGUGACAAGCUGUCGUGGCU
UUCAAGUAGGUAUGACGUUGGCGAGUACAuu
Divalent COPI_BP_1448/1490
(SEQ ID NO: ______)
GGAAGAAUCCUCAUACCUUCUUCAAGUGAAGGUAUGACGUUGGCGAC
UUCAAGUGCUGCCGACUCGGAUUCUUCCuu

A linked set including the MV-RNA above is shown below. Loops to be replaced using this invention (underlined):

(SEQ ID NO: ______)
GGUAUCGUACUCCCUUGUGUUUCAAGUACACAAGGGAAGAGAGAGUCGAU
UCAAGUUGUGUUCUCGUGUACGAUACCuuAAGCUGCCCGAGCAUAAGAUU
UCAAGUAUCUUAUGCUUAACCAAUCUUUCAAGUGGGGUGGUUGGCGGGCA
GCUUuuCGGCAACUACUCUUCCCUUGUUCAAGUCAAGGGAAGAGAGAGUC
GAGGUUCAAGUCAGUGACUUUGUAGUUGCCGuuAGUCCCAUAUUUUGACG
AGGUUUCAAGUACGUUGUUAGAGCCGUGCAUCUUCAAGUGAUGUAUGGCU
GUGUGGGAUUaaUGGCGUUCUCCGGCAUCGAAcUCAAGUUUCGAUGCCGU
CUUCUGUGAUUUCAAGUAUCAUAGAAGGAGAACGCCAuuUGCGACACUUU
CUAUGAUUUGCAAGUAAUCAUAGAAGGAGAACGCCAUUCAAGUUGGAGUU
CUCAAGUGUCGCAuuUCAUAUCUACCGACAGCUUGUUCAAGUCAAGCUGU
CGUGGAGCUGAUGUUCAAGUCAACAGUUUCUGGUAGAUAUGAuuGGAAGA
AUCCUCAUACCUUCUUCAAGUGAAGGUAUGACGUUGGCGACUUCAAGUGC
UGCCGACUCGGAUUCUUCCuuUGUAUUCGCCUUGUCACCAUUCAAGUUGG
UGACAAGCUGUCGUGGCUUUCAAGUAGGUAUGACGUUGGCGAGUACAuu

Design New, or Insert Pre-Existing Aptamer/Intramer Sequences into MV-RNA Loops

Using SELEX, one skilled in the art can design aptamer/intramer sequence specific for surface moieties. However, this can be costly and time consuming. An interim design allowing rapid testing of numerous surface substance candidates using well-studied aptamers with suitable pK from the public domain is provided [FIG. 12]. Aptamer sequences and surface substance sequences are not the subject of this invention, but this invention provides a useful tool making programmable nanoparticle surfaces whether derived from known or novel aptamer/moiety relationships. The BIV and TAT aptamer were chosen as suitable aptamers with an adequate disassociation constant (<60 nM & <600 nM) to drive surface assembly, then adapted using the methods and designs of this invention. The TAT and BIV aptamer sequences are shown below.

HIV-1 Tat,
(SEQ ID NO: ; structure shown below)
CCAGAUCUGAGC [symbol] GCUCUCUGG

BIV TAR,
(SEQ ID NO: ; structure shown below)
GAGGCGGUGAACUUGGAAUCCCACAAGGGCG

Then, mixed 16 bp stem for each aptamer were added between MVs.

TAT-Aptamer Stems for this example are shown below:

TAT Aptamer Stem Set 1:

(TAT Aptamer Stem 1a; SEQ ID NO:)
AGCCGGGCAGCUCCGACCAGAUCUGAGCCUGGGAGCUCUCUGGUCGGAG
CUGCCCGGCUuu
(TAT Aptamer Stem 1b; SEQ ID NO:)
CAGGGAGCUGCGGCGUCCAGAUCUGAGCCUGGGAGCUCUCUGGACGCCG
CAGCUCCCUGuu
(TAT Aptamer Stem 1c; SEQ ID NO:)
GACCCCGCCGUAGCCACCAGAUCUGAGCCUGGGAGCUCUCUGGUGGCUA
CGGCGGGGUCuu

TAT Aptamer Stem Set 2 (with different stem composition)

(TAT Aptamer Stem 2a; SEQ ID NO:)
CGACGGGCAGCUCGGACCAGAUCUGAGCCUGGGAGCUCUCUGGUCCGAG
CUGCCCGUCGuu
(TAT Aptamer Stem 2b; SEQ ID NO:)
GACGGAUCUGCGGCGUCCAGAUCUGAGCCUGGGAGCUCUCUGGACGCCG
CAGAUCCGUCuu
(TAT Aptamer Stem 2c; SEQ ID NO:)
GCCAUUGCCGUAGCCACCAGAUCUGAGCCUGGGAGCUCUCUGGUGGCUA
CGGCAAUGGCuu

BIV-Aptamer Stems for this example are shown below:

BIV-Aptamer Stem Set 1:

(BIV Aptamer Stem 1a; SEQ ID NO:)
AGCCGGGCAGCUCCGAGAGGCGGUGAACUUGGAAUCCCACAAG
GGCGUCGGAGCUGCCCGGCUuu
(BIV Aptamer Stem 1b; SEQ ID NO:)
CAGGGAGCUGCGGCGUGAGGCGGUGAACUUGGAAUCCCACAAG
GGCGACGCCGCAGCUCCCUGuu
(BIV Aptamer Stem 1c; SEQ ID NO:)
GACCCCGCCGUAGCCAGAGGCGGUGAACUUGGAAUCCCACAAG
GGCGUGGCUACGGCGGGGUCuu

BIV-Aptamer Stem Set 2 (with different stem composition):

(BIV Aptamer Stem 2a; SEQ ID NO:)
CGACGGGCAGCUCGGAGAGGCGGUGAACUUGGAAUCCCACAAG
GGCGUCCGAGCUGCCCGUCGuu
(BIV Aptamer Stem 2b; SEQ ID NO:)
GACGGAUCUGCGGCGUGAGGCGGUGAACUUGGAAUCCCACAAG
GGCGACGCCGCAGAUCCGUCuu
(BIV Aptamer Stem 2c; SEQ ID NO:)
GCCAUUGCCGUAGCCAGAGGCGGUGAACUUGGAAUCCCACAAG
GGCGUGGCUACGGCAAUGGCuu

The resulting TAT Aptamer Nanoparticle is shown below:

>FAW-4 (20 MV's), 3 target genes, 1860 nt
transcript
(SEQ ID NO: ; structure shown below)
CGCGCGTAATACGACTCACTATAGGGGC [symbol] GCAACGCCGGUCCC
UUCAGUGGCCGCUGGAUGUUUCAAGUACGUCCGGCACGCUCGUAGAGUU
CAAGUCUGUGUGAGCUCGUUGAUGGuuGGUAUCGUACUCCCUUGUGUUU
CAAGUACACAAGGGAAGAGAGAGUCGAUUCAAGUUGUGUUCUCGUGUAC
GAUACCuuAAGCUGCCCGAGCAUAAGAUUUCAAGUAUCUUAUGCUUAAC
CAAUCUUUCAAGGGGGGUGGUUGGCGGGCAGCUUuuAGCCGGGCAGCUC
CGACCAGAUCUGAGC [symbol] GCUCUCUGGUCGGAGCUGCCCGGCUu
uCGGCAACUACUCUUCCCUUGUUCAAGUCAAGGGAAGAGAGAGUCGAGG
UUCAAGUCAGUGACUUUGUAGUUGCCGuuCAGGGAGCUGCGGCGUCCAG
AUCUGAGC [symbol] GCUCUCUGGACGCCGCAGCUCCCUGuuUGCGAC
ACUUUCUAUGAUUUUCAAGUAAUCAUAGAAGGAGAACGCCAUUCAAGUU
GGAGUUCUCAAGAGACGCAuuUCAUAUCUACCGACAGCUUGUUCAAGUC
AAGCUGUCGUGGAGCUGAUGUUCAAGUCAACAGUUUCUGGUAGAUAUGA
uuGGAAGAAUCCUCAUACCUUCUUCAAGUGAAGGUAUGACGUUGGCGAC
UUCAAGUGCUGCCGACUCGGAUUCUUCCUUGACCCCGCCGUAGCCACCA
GAUCUGAGC [symbol] GCUCUCUGGUGGCUACGGCGGGGUCuuGUAUU
CGCCUUGUCACCAUUCAAGUUGGUGACAAGCUGUCGUGGCUUUCAAGUA
GGUAUGACGUUGGCGAGUACAuuCCUUCAGUGGCCGCUGGAUGUUUCAA
GUACGUCCGGCACGCUCGUAGAGUUCAAGUCUGUGUGAGCUCGUUGAUG
GuuGGUAUCGUACUCCCUUGUGUUUCAAGUACACAAGGGAAGAGAGAGU
CGAUUCAAGUUGUGUUCUCGUGUACGAUACCuuAAGCUGCCCGAGCAUA
AGAUCUCAAUUAUCUUAUGCUUAACCAAUCUUUCAAGUGGGGUGGUUGG
CGGGCAGCUUuuCGACGGGCAGCUCGGACCAGAUCUGAGC [symbol]
GCUCUCUGGUCCGAGCUGCCCGUCGuuCGGCAACUACUCUUCCCUUGUU
CAAGUCAAGGGAAGAGAGAGUCGAGGUUCAAGUCAGUGACUUUGUAGUU
GCCGuuAGUCCCAUAUUUUGACGAGGUUUCAAGUACGUUGUUAGAGCCG
UGCAUCUUCAAGOGAUGUAUGGCUGUGUGGGAUUaaUGGCGUUCUCCGG
CAUCGAACUCAAGUUUCGAUGCCGUCUUCUGUGAUUUCAAGUAUCAUAGA
AGGAGAACGCCAuuGACGGAUCUGCGGCGUCCAGAUCUGAGG [symbol]
CUCUCUGGACGCCGCAGAUCCGUCuuUGCGACACUUUCUAUGAUUUUCA
AGUAAUCAUAGAAGGAGAACGCCAUUCAAGUUGGAGUUCUCAAGUGUCG
CAuuUCAUAUCUACCGACAGCUUGUUCAAGUCAAGCUGUCGUGGAGCUG
AUGUUCAAGUCAACAGUUUCUGGUAGAUAUGAuuGGAAGAAUCCUCAUA
CCUUCUUCAAGUGAAGGUAUGACGUUGGCGACUUCAAGUGCUGCCGACU
CGGAUUCUUCCuuGCCAUUGCCGUAGCCACCAGAUCUGAGC [symbol]
GCUCUCUGGUGGCUACGGCAAUGGCuuGUAUUCGCCUUGUCACCAUUCA
AGUUGGUGACAAGCUGUCGUGGCUUUCAAGUAGGUAUGACGUUGGCGAG
UACAuuGACCGGCGTTGCCtTAGGCCGGTACCGCACCGACCAGAAUCAU
GCAAGUGCGUAAGAUAGUCGCGGGCCGGGGCGTCGACAAGCGCCGacaa
cCGGCGCTTTTTTtG

Add aptamer/intramer invention to the MV-RNA loop sequences

In support of the aptamer plurality used in this invention, aptamers were directly incorporated into each MV-RNA [FIG. 4a]. Of the 9 MV-RNA in this set, the first 3 receive TAT aptamers, the remaining 9 received BIV aptamers for a total of 18 aptamers, as shown below.

(SEQ ID NO: ; structure shown below)
GGUAUCGUACUCCCUUGUGUCCAGAUCUGAGC [symbol] GCUCUCUGG
ACACAAGGGAAGAGAGAGUCGACCAGAUCUGAGC [symbol] GCUCUCU
GGUGUGUUCUCGUGUACGAUACCuuAAGCUGCCCGAGCAUAAGAUCCAG
AUCUGAGC [symbol] GCUCUCUGGAUCUUAUGCUUAACCAAUCUCCAG
AUCUGAGC [symbol] GCUCUCUGGGGGGUGGUUGGCGGGCAGCUUuuC
GGCAACUACUCUUCCCUUGCCAGAUCUGAGC [symbol] GCUCUCUGGC
AAGGGAAGAGAGAGUCGAGGCCAGAUCUGAGC [symbol] GCUCUCUGG
CAGUGACUUUGUAGUUGCCGuuAGUCCCAUAUUUUGACGAGGUGAGGCG
GUGAACUUGGAAUCCCACAAGGGCGACGUUGUUAGAGCCGUGCAUCGAG
GCGGUGAACUUGGAAUCCCACAAGGGCGGAUGUAUGGCUGUGUGGGAUU
aaUGGCGUUCUCCGGCAUCGAAGAGGCGGUGAACUUGGAAUCCCACAAG
GGCGUUCGAUGCCGUCUUCUGUGAUGAGGCGGUGAACUUGGAAUCCCAC
AAGGGCGAUCAUAGAAGGAGAACGCCAuuUGCGACACUUUCUAUGAUUG
AGGCGGUGAACUUGGAAUCCCACAAGGGCGAUCAUAGAAGGAGAACGCC
AGAGGCGGUGAACUUGGAAUCCCACAAGGGCGUGGAGUUCUCAAGUGUC
GCAuuUCAUAUCUACCGACAGCUUGGAGGCGGUGAACUUGGAAUCCCAC
AAGGGCGCAAGCUGUCGUGGAGCUGAUGGAGGCGGUGAACUUGGAAUCC
CACAAGGGCGCAACAGUUUCUGGUAGAUAUGAuuGGAAGAAUCCUCAUA
CCUUCUUCAAGUGAAGGUAUGACGUUGGCGACGAGGCGGUGAACUUGGA
AUCCCACAAGGGCGGCUGCCGACUCGGAUUCUUCCuuUGUAUUCGOCUU
GUCACCAGAGGCGGUGAACUUGGAAUCCCACAAGGGCGUGGUGACAAGC
UGUCGUGGCUGAGGCGGUGAACUUGGAAUCCCACAAGGGCGAGGUAUGA
CGUUGGCGAGUACAuu

An exemplar co-fold notation of SEQ ID NO: is shown below:

(((((((((((((((((((((...((((....))))))))))))))))))...(((((.((((((((...((((......)))))))))).)) )))))..))))))))))..((((((((((.(((((((((((...((((......))))))))))))))))).((((((..(((((((...((((......)))))))))).. )))))).))))))))))..((((((((((.(((((((((((...((((......)))))))))))))))))..(((((((..((((((...((((......))))))))).. ))))))))))))))))).((((((((((((.((......((..((((........))))..)))).))))))))((((((((((..(.(..((....(((....))))).. ).).)))))))).))))))))).(((((((((((..(((((.((((((((((...(((((..(((....)))..)))))))))).))))).)))))....((((..((((... .....))))..).)))......)))))))))))..((((((.(((((((((((((....((....))......(((....))))))))))))))))....((((...))))....((((((((..((((......))))..))).)))))))))))..(((((((((((..((((((.(.(((.(((..((((........))))..)))..)))....).)))))).. ((((((.((( ..((((........))))..)))....)))))))))))))))))..((((((((((.(((((((((.......))))))))..(((((.....(((.((...((((........))) .)).)))))))))..))))))))))..((((((((((...(((((......))))).((..((((.(.(((.((....)).))).).)))))..))...((.(((((.(...(((....)))).)))).).))...))))))))))..

Preliminary Construct for the Production of Multi-Part Surfaces with a 1/3 to 2/3 Aptamer Ratios.

The resulting sequence was prepared for cloning into pBluescript and can be used for in-vitro transcription, intracellular E. coli production, or shuttling to a different organism, shown below.

>FAW-5 (9 MV's), (6 TAT-APTAMERS, 11 BIV-APTAMERS),
3 target genes, nt transcript
(SEQ ID NO:)
CGCGCGTAATACGACTCACTATAGGGGC [symbol] GCAACGCCGGUCGGU
AUCGUACUCCCUUGUGUCCAGAUCUGAGC [symbol] GCUCUCUGGAC
ACAAGGGAAGAGAGAGUCGACCAGAUCUGAGC [symbol] GCUCUCUG
GUGUGUUCUCGUGUACGAUACCuuAAGCUGCCCGAGCAUAAGAUCCAGA
UCUGAGC [symbol] GCUCUCUGGAUCUUAUGCUUAACCAAUCUCCAGA
UCUGAGC [symbol] GCUCUCUGGGGGGUGGUUGGCGGGCAGCUUuuCG
GCAACUACUCUUCCCUUGCCAGAUCUGAGC [symbol] GCUCUCUGGCA
AGGGAAGAGAGAGUCGAGGCCAGAUCUGAGC [symbol] UCUCUGGCAG
UGACUUUGUAGUUGCCGuuAGUCCCAUAUUUUGACGAGGUGAGGCGGUG
AACUUGGAAUCCCACAAGGGCGACGUUGUUAGAGCCGUGCAUCGAGGCG
GUGAACUUGGAAUCCCACAAGGGCGGAUGUAUGGCUGUGUGGGAUUaaU
GGCGUUCUCCGGCAUCGAAGAGGCGGUGAACUUGGAAUCCCACAAGGGC
GUUCGAUGCCGUCUUCUGUGAUGAGGCGGUGAACUUGGAAUCCCACAAG
GGCGAUCAUAGAAGGAGAACGCCAuuUGCGACACUUUCUAUCAUUGAGG
CGGUGAACUUGGAAUCCCACAAGGGCGAUCAUAGAAGGAGAACGCCAGA
GGCGGUGAACUUGGAAUCCCACAAGGGCGUGGAGUUCUCAAGUGUCGCA
uuUCAUAUCUACCGACAGCUUGGAGGCGGUGAACUUGGAAUCCCACAAG
GGCGCAAGCUGUCGUGGAGCUGAUGGAGGCGGUGAACUUGGAAUCCCAC
AAGGGCGGAACAGUUUCUGGUAGAUAUGAuuGGAAGAAUCCUCAUACCU
UCUUCAAGUGAAGGUAUGACGUUGGCGACGAGGCGGUGAACUUGGAAUC
CCACAAGGGCGGCUGCCGACUCGGAUUCUUCCuuUGUAUUCGCCUUGUC
ACCAGAGGCGGUGAACUUGGAAUCCCACAAGGGCGUGGUGACAAGCUGU
CGUGGCUGAGGCGGUGAACUUGGAAUCCCACAAGGGCGAGGUAUGACGU
UGGCGAGUACAuuGACCGGCGTTGCCtTAGGCCGGTACCGCACCGACCA
GAAUCAUGCAAGUGCGUAAGAUAGUCGCGGGCCGGGGCGTCGACAAGCG
CCGacaacCGGCGCTTTTTTtG

Increasing the MV-RNA plurality to meet higher aptamer ratios.

In support of the aptamer plurality used in this invention, aptamers were directly incorporated into each MV-RNA [FIG. 4a]. Of the 9 MV-RNA in this set, the first 3 receive TAT aptamers, the remaining 11 received BIV aptamers for a total of 22 aptamers (6 TAT-APTAMERS/22 BIV-APTAMERS)

1X TAT-APTAMER REGION:
(SEQ ID NO:)
GGUAUCGUACUCCCUUGUGUCCAGAUCUGAGC [symbol] GCUCUCUGGA
CACAAGGGAAGAGAGAGUCGACCAGAUCUGAGC [symbol] GCUCUCUGG
UGUGUUCUCGUGUACGAUACCuuAAGCUGCCCGAGCAUAAGAUCCAGAUC
UGAGC [symbol] GCUCUCUGGAUCUUAUGCUUAACCAAUCUCCAGAUCU
GAGC [symbol] GCUCUCUGGGGGGUGGUUGGCGGGCAGCUUuuCGGCAA
CUACUCUUCCCUUGCCAGAUCUGAGC [symbol] GCUCUCUGGCAAGGGA
AGAGAGAGUCGAGGCCAGAUCUGAGC [symbol] GCUCUCUGGCAGUGAC
UUUGUAGUUGCCGuu
2X BIV-APTAMER REGION:
(SEQ ID NO:)
AGUCCCAUAUUUUGACGAGGUGAGGCGGUGAACUUGGAAUCCCACAAGGG
CGACGUUGUUAGAGCCGUGCAUCGAGGCGGUGAACUUGGAAUCCCACAAG
GGCGGAUGUAUGGGUGUGUGGGAUUaaUGGCGUUCUCCGGCAUCGAAGAG
GCGGUGAACUUGGAAUCCCACAAGGGCGUUCGAUGCCGUCUUCUGUGAUG
AGGCGGUGAACUUGGAAUCCCACAAGGGCGAUCAUAGAAGGAGAACGCCA
uuUGCGACACUUUCUAUGAUUGAGGCGGUGAACUUGGAAUCCCACAAGGG
CGAUCAUAGAAGGAGAACGCCAGAGGCGGUGAACUUGGAAUCCCACAAGG
GCGUGGAGUUCUCAAGUGUCGCAuuUCAUAUCUACCGACAGCUUGGAGGC
GGUGAACUUGGAAUCCCACAAGGGCGCAAGCUGUCGUGGAGCUGAUGGAG
GCGGUGAACUUGGAAUCCCACAAGGGCGCAACAGUUUCUGGUAGAUAUGA
uuGGAAGAAUCCUCAUACCUUCUUCAAGUGAAGGUAUGACGUUGGCGACG
AGGCGGUGAACUUGGAAUCCCACAAGGGCGGCUGCCGACUCGGAUUCUUC
CuuUGUAUUCGCCUUGUCACCAGAGGCGGUGAACUUGGAAUCCCACAAGG
GCGUGGUGACAAGCUGUCGUGGCUGAGGCGGUGAACUUGGAAUCCCACAA
GGGCGAGGUAUGACGUUGGCGAGUACAuu
(SEQ ID NO:)
AGUCCCAUAUUUUGACGAGGUGAGGCGGUGAACUUGGAAUCCCACAAGGG
CGACGUUGUUAGAGCCGUGGAUCGAGGCGGUGAACUUGGAAUCCCACAAG
GGCGGAUGUAUGGCUGUGUGGGAUUaaUGGCGUUCUCCGGCAUCGAAGAG
GCGGUGAACUUGGAAUCCCACAAGGGCGUUCGAUGCCGUCUUCUGUGAUG
AGGCGGUGAACUUGGAAUCCCACAAGGGCGAUCAUAGAAGGAGAACGCCA
uuUGCGACACUUUCUAUGAUUGAGGCGGUGAACUUGGAAUCCCACAAGGG
CGAUCAUAGAAGGAGAACGCCAGAGGCGGUGAACUUGGAAUCCCACAAGG
GCGUGGAGUUCUCAAGUGUCGCAuuUCAUAUCUACCGACAGCUUGGAGGC
GGUGAACUUGGAAUCCCACAAGGGCGCAAGCUGUCGUGGAGCUGAUGGAG
GCGGUGAACUUGGAAUCCCACAAGGGCGCAACAGUUUCUGGUAGAUAUGA
uuGGAAGAAUCCUCAUACCUUCUUCAAGUGAAGGUAUGACGUUGGCGACG
AGGCGGUGAACUUGGAAUCCCACAAGGGCGGCUGCCGACUCGGAUUCUUC
CuuUGUAUUCGCCUUGUCACCAGAGGCGGUGAACUUGGAAUCCCACAAGG
GCGUGGUGACAAGCUGUCGUGGCUGAGGCGGUGAACUUGGAAUCCCACAA
GGGCGAGGUAUGACGUUGGCGAGUACAuu

Final Construct for the Production of Multi-Part Surfaces with a 1/3 to 2/3 Aptamer Ratios.

The resulting sequence was prepared for cloning into pBluescript and can be used for in-vitro transcription, intracellular E. coli production, or shuttling to a different organism.

1/3 Tat-Aptamer & 2/3 Biv-Aptamer Nanoparticle: (6 Tat-Aptamers, 22 Biv-Aptamers)

>FAW-6 (20 MV's), 3 target genes
(SEQ ID NO:)
CGCGCGTAATACGACTCACTATAGGGGC [symbol] GCAACGCCGGUCGGUA
UCGUACUCCCUUGUGUCCAGAUCUGAGC [symbol] GCUCUCUGGACACAA
GGGAAGAGAGAGUCGACCAGAUCUGAGC [symbol] GCUCUCUGGUGUGUU
CUCGUCUACGAUACCuuAAGCUGCCCGAGCAUAAGAUCCAGAUCUGAGC [symbol]
GCUCUCUGGAUCUUAUGCUUAACCAAUCUCCAGAUCUGAGC [symbol]
GCUCUCUGGGGGGUGGUUGGCGGGCAGCUUuuCGGCAACUACU
CUUCCCUUGCCAGAUCUGAGC [symbol] GCUCUCUGGCAAGGGAAGAGAG
AGUCGAGGCCAGAUCUGAGC [symbol] GCUCUCUGGCAGUGACUUUGUAG
UUGCCGuuAGUCCCAUAUUUUGACGAGGUGAGGCGGUGAACUUGGAAUCCC
ACAAGGGCGACGUUGUUAGAGCCGUGCAUCGAGGCGGUGAACUUGGAAUCC
CACAAGGGCGGAUGUAUGGCUGUGUGGGAUUaaUGGCGUUCUCCGGCAUCG
AAGAGGCGGUGAACUUGGAAUCCCACAAGGGCGUUCGAUGCCGUCUUCUGU
GAUGAGGCGGUGAACUUGGAAUCCCACAAGGGCGAUCAUAGAAGGAGAACG
CCAuuUGCGACACUUUCUAUGAUUGAGGCGGUGAACUUGGAAUCCCACAAG
GGCGAUCAUAGAAGGAGAACGCCAGAGGCGGUGAACUUGGAAUCCCACAAG
GGCGUGGAGUUCUCAAGUGUCGCAuuUCAUAUCUACCGACAGCUUGGAGGC
GGUGAACUUGGAAUCCCACAAGGGCGCAAGCUGUCGUGGAGCUGAUGGAGG
CGGUGAACUUGGAAUCCCACAAGGGCGCAACAGUUUCUGGUAGAUAUGAuu
GGAAGAAUCCUCAUACCUUCUUCAAGUGAAGGUAUGACGUUGGCGACGAGG
CGGUGAACUUGGAAUCCCACAAGGGCGGCUGCCGACUCGGAUUCUUCCuuU
GUAUUCGCCUUGUCACCAGAGGCGGUGAACUUGGAAUCCCACAAGGGCGUG
GUGACAAGCUGUCGUGGCUGAGGCGGUGAACUUGGAAUCCCACAAGGGCGA
GGUAUGACGUUGGCGAGUACAuuAGUCCCAUAUUUUGACGAGGUGAGGCGG
UGAACUUGGAAUCCCACAAGGGCGACGUUGUUAGAGCCGUGGAUCGAGGCG
GUGAACUUGGAAUCCCACAAGGGCGGAUGUAUGGCUGUGUGGGAUUaaUGG
CGUUCUCCGGCAUCGAAGAGGCGGUGAACUUGGAAUCCCACAAGGGCGUUC
GAUGCCGUCUUCUGUGAUGAGGCGGUGAACUUGGAAUCCCACAAGGGCGAU
CAUAGAAGGAGAACGCCAuuUGCGACACUUUCUAUGAUUGAGGCGGUGAAC
UUGGAAUCCCACAAGGGCGAUCAUAGAAGGAGAACGCCAGAGGCGGUGAAC
UUGGAAUCCCACAAGGGCGUGGAGUUCUCAAGUGUCGCAuuUCAUAUCUAC
CGACAGCUUGGAGGCGGUGAACUUGGAAUCCCACAAGGGCGCAAGCUGUCG
UGGAGCUGAUGGAGGCGGUGAACUUGGAAUCCCACAAGGGCGCAACAGUUU
CUGGUAGAUAUGAuuGGAAGAAUCCUCAUACCUUCUUCAAGUGAAGGUAUG
ACGUUGGCGACGAGGCGGUGAACUUGGAAUCCCACAAGGGCGGCUGCCGAC
UCGGAUUCUUCCuuUGUAUUCGCCUUGUCACCAGAGGCGGUGAACUUGGAA
UCCCACAAGGGCGUGGUGACAAGCUGUCGUGGCUGAGGCGGUGAACUUGGA
AUCCCACAAGGGCGAGGUAUGACGUUGGCGAGUACAuuGACCGGCGTTGCC
tTAGGCCGGTACCGCACCGACCAGAAUCAUGCAAGUGCGUAAGAUAGUCGC
GGGCCGGGGCGTCGACAAGCGCCGacaacCGGCGCTTTTTTtG

Surface Moieties Derived from Natural Sources or with Specific Function in Fall armyworm.

As a non-limiting example, any two of these BIV or TAT adapted polypeptides can be used for testing as multi-part nanoparticle surfaces using the designs and methods of this invention example.

Plant Derived Surface Polypeptides:

TAT_MAIZE_RIP2_N
(SEQ ID NO:)
MHKHKYGRKKRRQRRRaaaaMAEPNPELSGLITQTKKKNIVPKFTEIF
PVEDTAYPYSAFITSVRKEVIKYCTNHTGIVQPVLPLEKNVPELWFYTE
LKTKTRSITLAIRMDNLYLVGFRTPGGVWWEFGKDGDTHLLDDNAK
WLGFGGRYQDLIGSKGLETVTMGRAEMTTAVNYLAKKTTHHHHHH
TAT_MAIZE_RIP2_C
(SEQ ID NO:)
MHKHKYGRKKRRQRRRaaaaADPKAEEKSNLAKLVIMVCEGLRFFTV
SRKVDEGFKKPQAVTISALEGKQVQKWDRISKAVFRWAVDPTAEIPD
MKDLGIKDKNAAAQIVALVKDQNHHHHHH
BIV_RIP2_N
(SEQ ID NO:)
MHKHKSGPRPRGTRGKGRRIRRaaaaMAEPNPELSGLITQTKKKNIVP
KFTEIFPVEDTAYPYSAFITSVRKEVIKYCTNHTGIVQPVLPLEKNVPEL
WFYTELKTKTRSITLAIRMDNLYLVGFRTPGGVWWEFGKDGDTHLLD
DNAKWLGFGGRYQDLIGSKGLETVTMGRAEMTTAVNYLAKKTTHHH
HHH
BIV_RIP2_C
(SEQ ID NO:)
MHKHKSGPRPRGTRGKGRRIRRaaaaADPKAEEKSNLAKLVIMVCEG
LRFFTVSRKVDEGFKKPQAVTISALEGKQVQKWDRISKAVFRWAVDP
TAEIPDMKDLGIKDKNAAAQIVALVKDQNHHHHHH
TAT_SNOW_LECTIN
(SEQ ID NO:)
MHKHKYGRKKRRQRRRaaaaDNILYSGETLSTGEFLNYGSFVFIMQE
DCNLVLYDVDKPIWATNTGGLSRSCFLSMQTDGNLWYNPSNKPIWA
SNTGGQNGNYVCILQKDRNVVIYGTDRWATGHHHHHH
BIV_SNOW_LECTIN
(SEQ ID NO:)
MHKHKSGPRPRGTRGKGRRIRRaaaaDNILYSGETLSTGEFLNYGSF
VFIMQEDCNLVLYDVDKPIWATNTGGLSRSCFLSMQTDGNLVVYNPS
NKPIWASNTGGQNGNYVCILQKDRNVVIYGTDRWATGHHHHHH
TAT-Maize mir1
(SEQ ID NO:)
YGRKKRRQRRRMRPTRSAVSATALLLLAVALALAATAAARHSYTTTT
TRVPAPAERADEEVRRMYEAWKSKHGRGGSSNDDCDMAPGDDEQ
EEEDRRLRLEVFRDNLRYIDAHNAEADAGLHTFRLGLTPFADLTLEEY
RGRVLGFRARGRRSGARYGSGYSVRGGDLPDAIDWRQLGAVTEVK
DQQQCGGCWAFSAVAAIEGVNAIATGNLVSLSEQEIIDCDAQDSGCD
GGQMENAFRFVIGNGGIDTEADYPFIGTDGTCDASKEKNEKVATIDG
LVEVASNNETALQEAVAIQPVSVAIDASGRAFQHYSSGIFNGPCGTSL
DHGVTAVGYGSESGKDYWIVKNSWSASWGEAGYIRMRRNVPRPTG
KCGIAMDASYPVKDTYHPGTGTATARAAAMDVIKMVLAHHHHHH

Gut Receptor Polypeptides:

TAT_CRY2
(SEQ ID NO:)
YGRKKRRQRRRCHLPRLPQCCHLPRLPQC
ENDOSOMAL ESCAPE POLYPEPTIDES:
CM-TAT
(SEQ ID NO:)
KWKLFKKIGAVLKVLTTGYGRKKRRQRRR
E5-TAT
(SEQ ID NO:)
GLFEAIAEFIENGWEGLIEGWYGGRKKRRQRRR

Insect Virus Polypeptides:

BIV_DENSO-VP4
(SEQ ID NO:)
MHKHKSGPRPRGTRGKGRRIRRaaaaMSLPGTGSGTSSGGGNTSG
QEVYVIPRPFSNFGKKLSTYTKSHKFMIFGLANNVIGPTGTGTTAVNR
LITTCLAEIPWQKLPLYMNQSEFDLLPPGSRVVECNVKVIFRTNRIAFE
TSSTATKQATLNQISNLQTAVGLNKLGWGIDRSFTAFQSDQPM1PTAT
SAPKYEPITGTTGYRGMIADYYGADSTNDAAFGNAGNYPHHQVGSF
TFIQNYYCMYQQTNQGTGGWPCLAEHLQQFDSKTVNNQCLIDVTYK
PKMGLIKPPLNYKIIGQPTAKGTISVGDNLVNMRGAVVINPPEATQSVT
ESTHNLTRNFPANLFNIYSDIEKSQILHKGPWGHENPQIQPSVHIGIQA
VPALTTGALLVNSSPLNSWTDSMGYIDVMSSCTVMESQPTHFPFSTD
ANTNPGNTIYRINLTPNSLTSAFNGLYGNGATLGNVHHHHHH
TAT_DENSO-VP4
(SEQ ID NO:)
MHKHKYGRKKRRQRRRaaaaMSLPGTGSGTSSGGGNTSGQEVYVIP
RPFSNFGKKLSTYTKSHKFMIFGLANNVIGPTGTGTTAVNRLITTCLAE
IPWQKLPLYMNQSEFDLLPPGSRVVECNVKVIFRTNRIAFETSSTATK
QATLNQISNLQTAVGLNKLGWGIDRSFTAFQSDQPMIPTATSAPKYE
PITGTTGYRGMIADYYGADSTNDAAFGNAGNYPHHQVGSFTFIQNYY
CMYQQTNQGTGGWPCLAEHLQQFDSKTVNNQCLIDVTYKPKMGLIK
PPLNYKIIGQPTAKGTISVGDNLVNMRGAWINPPEATQSVTESTHNL
TRNFPANLFNIYSDIEKSQILHKGPWGHENPQIQPSVHIGIQAVPALTT
GALLVNSSPLNSWTDSMGYIDVMSSCTVMESQPTHFPFSTDANTNP
GNTIYRINLTPNSLTSAFNGLYGNGATLGNVHHHHHH

### CITATIONS

Non-Viral Polynucleotide Delivery Citations:
[POLY.1] M. J. Tiera et al, Curr. Gene Ther. 6 (2006) 59-71,13
[POLY.2] X. Gao et al, AAPS J. 9 (2007) E92-E104
[POLY.3] K. Lappalainen et al, Pharm. Res. 11 (1994) 1127-1131
[POLY.4] M. D. Brown et al, Int. J. Pharm. 229 (2001) 1-21; H. Hosseinkhani et al, Gene Ther. 11 (2004) 194-203.
[POLY.5] U. Lungwitz et al, Eur. J. Pharm. Biopharm. 60 (2005) 247-266
[POLY.6] Zheng 2012, 2013; Zhou 2013, Jensen 2013, Ding 2014
[POLY.8] Michael Wagner, Alexandra C. Rinkenauer, Anja Schallonab, Ulrich S. Schubert, Opposites attract: influence of the molar mass of branched poly(ethylene imine) on biophysical characteristics of siRNA-based polyplexese. RSC Adv., 2013,3, 12774-12785
[POLY.N] Laufer, S. D., Detzer, A., Sczakiel, G., and Restle, T. (2012). “Selected strategies for the delivery of siRNA in vitro and in vivo,” in RNA Technologies and Their Applications, eds V. A. Erdmann and J. Barciszewski (Heidelberg; Dorcrecht; London; New York, N.Y.: Springer), 39-45. doi: 10.1007/978-3-642-12168-5_2
[POLY.N] Lim, J. P., and Gleeson, P. A. (2011). Macropinocytosis: an endocytic pathway for internalising large gulps. Immunol. Cell Biol. 89, 836-843. doi: 10.1038/icb.2011.20
[POLY.N.1] Prokop., A., Iwasaki, Y., and Harada, A. (2014). Intracellular Delivery II. Fundamentals and Applications. Dordrecht; Heidelberg; New York, N.Y.; London: Springer.

Virus-Like Particle Citations:
[VLP.1] H. Fraenkel-Conrat, 1955
[VLP.2] Evolution and Protein Packaging of Small-Molecule RNA Aptamers Jolene L. Lau, Michael M. Baksh, Jason D. Fiedler, Steven D. Brown, Amanda Kussrow, Darryl J. Bornhop, Phillip Ordoukhanian, and M. G. Finn, ACS Nano 0 0 (proofing), DOI: 10.1021/nn2006927@proofing.
[VLP.3] Prel A, Caval V, Gayon R, et al. Highly efficient in vitro and in vivo delivery of functional RNAs using new versatile MS2-chimeric ret-rovirus-like particles. Mol Ther Methods Clin Dev. 2015; 2:15039.

Anti-Microbial Peptide Citations:
AMP.1, Zasloff et al. 2002
AMP.2, Nguyen et al. 2011
AMP.3, Hancock et al. 1997
AMP.4, Hancock and Lehrer et al. 1998
AMP.5, Agrios et al. 2005
AMP.6, Montesinos et al. 2007
AMP.7, Maloy et al. 1995
AMP.8, Ramamoorthy et al. 2006
AMP.9, Kang et al.2009
AMP.10, Park et al. 2002
AMP.11. Jung et al. 2012
AMP.12, Gordon et al. 2005
AMP.13, Oyston et al. 2009
AMP.14, Lupetti et al. 2000
AMP.15, Lupetti et al. 2003
AMP.16, Brouwer et al. 2011
AMP.17, Hwang et al. 1998
AMP.18, Nguyen et al. 2005
[AMP.13] De Lucca A J, Cleveland T E, Wedge D E. Plant-derived antifungal proteins and peptides. Can J Microbiol. 2005; 51: 1001-1014.
[AMP.14] De Lucca A J, Walsh T J. Antifungal peptides: novel therapeutic compounds against emerging pathogens. Antimicrob Agents Chemother. 1999; 43:1-11.
[AMP.15] Huynh Q K, Hironakan C M, Levinell E B, et al. Antifungal proteins from plants: purification, molecular cloning, and antifungal properties of chitinases from maize seed. J Biol Chem. 1992; 267: 6635-6640.
[AMP.16] Selitrennikoff C P. Antifungal proteins. Appl Environ Microbiol. 2001; 67: 2883-2894.
[AMP.17] Terras F R G, Schoofs H M E, De Bolle M F C, et al. Analysis of two novel classes of plant antifungal proteins from radish (Raphanus sativus L.) seeds. J Biol Chem. 1992; 267: 15301-15309.
[AMP.18] Terras F R G, Torrekens S, Van Leuven F, et al. A new family of basic cysteine-rich plant antifungal proteins from Brassicaceae species FEBS Lett. 1993; 316: 233-240.
[AMP.19] Vigers A J, Roberts W K, Selitrennikoff C P. A new family of plant antifungal proteins. Mol Plant Microbe Interact. 1991; 4: 315-323.
[AMP.20] Aley S B, Zimmerman M, Hetsko M, Selsted M E, Gillin F D. Killing of Giardia lamblia by cryptdins and cationic neutrophil peptides. Infect Immun. 1994; 62: 5397-5403.
[AMP.21] Kliger Y, Gallo S A, Peisajovich S G, et al. Mode of action of an antiviral peptide from HIV-1: inhibition at a post lipid-mixing stage. J Biol Chem. 2001; 276: 1391-1397.

Aptamer and Intramer Citations:
[APT.1] Yarus, M. Life from an RNA World: the ancestor within. Harvard University Press, New York, 2010.
[APT.2] Connell, G. J., Illangsekare, M., Yarus, M. Three small ribooligonucleotides with specific arginine sites. Biochemistry 32 (1993) 5497-5502.
[APT.3] Khvorova, A., Kwak, Y.-G., Tamkun, M., Majerfeld, I. and Yarus, M. RNAs that bind and change the permeability of phospholipid membranes. Proc. Natl. Acad. Sci. USA 96 (1999) 10649-10654.
[APT.4] Yarus, M. A specific amino acid binding site composed of RNA. Science 240 (1988) 1751-1758. 36 Vol. 16. No. 1. 2011 CELL. MOL. BIOL. LETT.
[APT.5] Roth, A., Winkler, W. C., Regulski, E. E., Lee, B. W. K., Lim, J., Jona, I., Barrick, J. E., Ritwik, A., Kim, J. N., Welz, R., lwata-Reuyl, D. and Breaker, R. R. A riboswitch selective for the queuosine precursor preQ1 contains an unusually small aptamer domain. Nat. Struct. Mol. Biol. 14 (2007) 308-317.
[APT.6] Spitale, R. C., Terelli, A. T., Krucinska, J., Bandarlan, V., Wedekind, J. E. The structural basis for recognition of the preQO metabolite by an unusually small riboswitch aptamer domain. J. Biol. Chem. 284 (2009) 11012-11016.
[APT.7] Ellington, A. D. and Szostak, J. W. In vitro selection of RNA molecules that bind specific ligands. Nature 346 (1990) 818-822.
[APT.8] Tuerk, C. and Gold, L. Systematic evolution of ligands by exponential enrichment: RNA ligands to bacteriophage T4 DNA-polymerase. Science 249 (1990) 505-510.
[APT.9] Janas, T., Widmann, J. J., Knight, R. and Yarus, M. Simple, recurrent RNA binding sites for L-arginine. RNA (2010) 805-816.

[APT.10] Bock, L. C., Griffin, L. C., Latham, J. A., Vermaas, E. H. and Toole, J. J. Selection of single-stranded DNA molecules that bind and inhibit human thrombin. Nature 355 (1992) 564-566.
[APT.11] Anderson, P. C. and Mecozzi, S. Unusually short RNA sequences: design of a 13-mer RNA that selectively binds and recognizes theophylline. J. Am. Chem. Soc. 127 (2005) 5290-5291.
[APT.12] Farokhzad, O. C., Cheng, J., Teply, B. A., Sherifi, I., Jon, S., Kantoff, P. W., Richie, J. P. and Langer, R. Targeted nanoparticle-aptamer bioconjugates for cancer chemotherapy in vivo. Proc. Natl. Acad. Sci. USA 103 (2006) 6315-6320.
[APT.13] Song, S., Wang, L., Li, J., Zhao, J. and Fan, C. Aptamer-based biosensors. Trends Anal. Chem. 27 (2008) 108-117.
[APT.14] Lee, J. O., So, H. M., Jeon, E. K., Chang, H., Won, K. and Kim, Y. H. Aptamers as molecular recognition elements for electrical nanobiosensors. Anal. Bioanal. Chem. 390 (2008) 1023-1032.
[APT.15] Barbas, A. S. and White, R. R. The development and testing for cancer. Curr. Opin. Investig. Drugs 10 (2009) 572-578.

Virology Citations:
[VIR.1] Dickson, A. M., and Wilusz, J. (2011). Strategies for viral RNA stability: live long and prosper. Trends Genet. 27, 286-293. doi: 10.1016/j.tig.2011.04.003
[VIR.1] Fenner, B. J., Goh, W., and Kwang, J. (2006). Sequestration and protection of double-stranded RNA by the betanodavirus B2 protein. J. Virol. 80, 6822-6833. doi: 10.1128/JVI.00079-06

Cell Penetrating Peptide Citations:
[CPP.1] A. Padhi, et al. Antimicrobial peptides and proteins in mycobacterial therapy: current status and future prospects Tuberculosis, 94 (2014), pp. 363-373.
[CPP.2] H. Buchwald, et al. Effects on GLP-1, PYY, and leptin by direct stimulation of terminal ileum and cecum in humans: implications for ileal transposition. Surg. Obes. Relat. Dis. (2014) http://dx.doi.orq/10.1016/j.soard.2014.01.032
[CPP.3] C. Giordano, et al. Neuroactive peptides as putative mediators of antiepileptic ketogenic diets. Front. Neurol., 5 (2014), p. 63.
[CPP.4] S. D. Robinson, et al. Diversity of conotoxin gene superfamilies in the venomous snail, Conus victoriae PLOS ONE, 9 (2014), p. e87648.
[CPP.1] Cermenati, G., Terracciano, I., Castelli, I., Giordana, B., Rao, R., Pennacchio, F., et al. (2011). The CPP Tat enhances eGFP cell internalization and transepithelial transport by the larval midgut of Bombyx mori (Lepidoptera, Bombycidae). J. Insect Physiol. 57, 1689-1697. doi: 10.1016/j.jinsphys.2011.09.004
[CPP.1] Chen, Y.-J., Liu, B. R., Dai, Y.-H., Lee, C.-Y., Chan, M.-H., Chen, H.-H., et al. (2012). A gene delivery system for insect cells mediated by arginine-rich cell-penetrating peptides. Gene 493, 201-210. doi: 10.1016/j.gene.2011.11.060
[CPP.1] Chugh, A., Eudes, F., and Shim, Y. S. (2010). Cell-penetrating peptides: nanocarrier for macromolecule delivery in living cells. IUBMB Life 62, 183-193. doi: 10.1002/iub.297
[CPP.1] Danielson, D. C., Sachrajda, N., Wang, W., Filip, R., and Pezacki, J. P. (2016). A novel p19 fusion protein as a delivery agent for short-interfering RNAs. Mol. Ther. Nucleic Acids 5:e303. doi: 10.1038/mtna.2016.14 de Lima, I. S. Jr., Degrande, P. E., Miranda, J. E., and dos Santos, W. J. (2013).
[CPP.1] Eguchi, A., Meade, B. R., Chang, Y.-C., Fredrickson, C. T., Willert, K., Puri, N., et al. (2009). Efficient siRNA delivery into primary cells by a peptide transduction domain-dsRNA binding domain fusion protein. Nat. Biotechnol. 27, 567-571. doi: 10.1038/nbt.1541
[CPP.1] El-Sayed, A., and Harashima, H. (2013). Endocytosis of gene delivery vectors: from clathrin-dependent to lipid raft-mediated endocytosis. Mol. Ther. 21, 1118-1130. doi: 10.1038/mt.2013.54
[CPP.1] Erazo-Oliveras, A., Muthukrishnan, N., Baker, R., Wang, T. Y., and Pellois, J. P. (2012). Improving the endosomal escape of cell-penetrating peptides and their cargos: strategies and challenges. Pharmaceuticals 5, 1177-1209. doi: 10.3390/ph5111177
[CPP.1] Hastie, K. M., Bale, S., Kimberlin, C. R., and Saphire, E. O. (2012). Hiding the evidence: two strategies for innate immune evasion by hemorrhagic fever viruses. Curr. Opin. Virol. 2, 151-156. doi: 10.1016/j.coviro.2012. 01.003
[CPP.1] Hughes, S. R., Dowd, P. F., and Johnson, E. T. (2012). Cell-penetrating recombinant peptides for potential use in agricultural pest control applications. Pharmaceuticals 5, 1054-1063. doi: 10.3390/ph5101054
[CPP.1] Kaplan, I. M., Wadia, J. S., and Dowdy, S. F. (2005). Cationic TAT peptide transduction domain enters cells by macropinocytosis. J. Control. Release 102, 247-253. doi: 10.1016/j.jconre1.2004.10.018
[CPP.1] Khalil, I. A., Kogure, K., Futaki, S., and Harashima, H. (2006). High density of octaarginine stimulates macropinocytosis leading to efficient intracellular trafficking for gene expression. J. Biol. Chem. 281, 3544-3551. doi: 10.1074/jbc. M503202200
[CPP.1] Kosuge, M., Takeuchi, T., Nakase, I., Jones, A. T., and Futaki, S. (2008). Cellular internalization and distribution of arginine-rich peptides as a function of extracellular peptide concentration, serum, and plasma membrane associated proteoglycans. Bioconjug. Chem. 19, 656-664. doi: 10.1021/bc700289w
[CPP.1] Liu, B. R., Liou, J. S., Huang, Y. W., Aronstam, R. S., and Lee, H. J. (2013). Intracellular delivery of nanoparticles and DNAs by IR9 cell-penetrating peptides. PLoS ONE 8:e64205. doi: 10.1371/journal.pone.00 64205
[CPP.1] Milletti, F. (2012). Cell-penetrating peptides: classes, origin, and current landscape. Drug Discov. Today 17, 850-860. doi: 10.1016/j.drudis.2012. 03.002
[CPP.1] Nakase, I., Niwa, M., Takeuchi, T., Sonomura, K., Kawabata, N., Koike, Y., et al. (2004). Cellular uptake of arginine-rich peptides: roles for macropinocytosis and actin rearrangement. Mol. Ther. 10, 1011-1022. doi: 10.1016/j.ymthe.2004.08.010
[CPP.1] Rinne, J., Albarran, B., Jylhävä, J., Ihalainen, T. O., Kankaanpäaä P., Hytonen, V. P., et al. (2007). Internalization of novel non-viral vector TAT-streptavidin into human cells. BMC Biotechnol. 7:1. doi: 10.1186/1472-6750-7-1
[CPP.1] Ryter, J. M., and Schultz, S. C. (1998). Molecular basis of double-stranded RNA-protein interactions: structure of a dsRNA-binding domain complexed with dsRNA. EMBO J. 17, 7505-7513. doi: 10.1093/emboj/17. 24.7505
[CPP.1] Tanaka, G., Nakase, I., Fukuda, Y., Masuda, R., Oishi, S., Shimura, K., et al. (2012). CXCR4 stimulates macropinocytosis: implications for cellular uptake of arginine-Tich cell-penetrating peptides and HIV. Chem. Biol. 19, 1437-1446. doi: 10.1016/j.chembio1.2012.09.011
[CPP.1] Tünnemann, G., Martin, R. M., Haupt, S., Patsch, C., Edenhofer, F., and Cardoso, M. C. (2006). Cargo-dependent mode of uptake and bioavailability of TAT-containing proteins and peptides in living cells. FASEB J. 20, 1775-1784. doi: 10.1096/fj.05-5523com
[CPP.1] Wadia, J. S., Stan, R. V., and Dowdy, S. F. (2004). Transducible TAT-HA fusogenic peptide enhances escape of TAT-fusion proteins after lipid raft macropinocytosis. Nat. Med. 10, 310-315. doi: 10.1038/nm996

Insect RNAi Citations:
[INSECT.1] Bally, J., McIntyre, G. J., Doran, R. L., Lee, K., Perez, A., Jung, H., et al. (2016). In-plant protection against Helicoverpa armigera by production of long hpRNA in Chloroplasts. Front. Plant Sci. 7:1453. doi: 10.3389/fpls.2016. 01453
[INSECT.1] Baum, J. A., Bogaert, T., Clinton, W., Heck, G. R., Feldmann, P., (lagan, O., et al. (2007). Control of coleopteran insect pests through RNA interference. Nat. Biotechnol. 25, 1322-1326. doi: 10.1038/nbt1359
[INSECT.1] Belles, X. (2010). Beyond Drosophila: RNAi in vivo and functional genomics in insects. Annu. Rev. Enthomol. 55, 111-128. doi: 10.1146/annurev-ento-1 12408-085301
[INSECT.1] Bolognesi, R., Ramaseshadri, P., Anderson, J., Bachman, P., Clinton, W., Flannagan, R., et al. (2012). Characterizing the mechanism of action of double-stranded RNA activity against western corn rootworm (Diabrotica virgifera virgifera LeConte). PLoS ONE 7:e47534. doi: 10.1371/journal.pone.0047534
[INSECT.1] Bronkhorst, A. W., Van Cleef, K. W., Venselaar, H., and Van Rij, R. P. (2014). A dsRNA-binding protein of a complex invertebrate DNA virus suppresses the Drosophila RNAi response. Nucleic Acids Res. 42, 12237-12248. doi: 10.1093/nar/gku910
[INSECT.1] Burand, J. P., and Hunter, W. B. (2013). RNAi: future in insect management. J. Invertebr. Pathol. 112, 68-74. doi: 10.1016/j.jip.2012.07.012
[INSECT.1] Evaluation of the boll weevil Anthonomus grandis boheman (Coleoptera: Curculionidae) suppression program in the State of Goiás, Brazil. Neotrop. Entomol. 42, 82-88. doi: 10.1007/s13744-012-0083-3
[INSECT.1] Firmino, A. A., Fonseca, F. C., Macedo, L. L., Coelho, R. R., Souza, J. R., Togawa, R. C., et al. (2013). Transcriptome analysis in Cotton Boll Weevil (Anthonomus grandis) and RNA interference in insect pests. PLoS ONE 8:e85079. doi: 10.1371/journal.pone.0085079
[INSECT.1] Hegedus, D., Erlandson, M., Gillott, C., and Toprak, U. (2009). New insights into peritrophic matrix synthesis, architecture, and function. Annu. Rev. Entomol. 54, 285-302. doi: 10.1146/annurev.ento.54.110807. 090559
[INSECT.1] Huvenne, H., and Smagghe, G. (2010). Mechanisms of dsRNA uptake in insects and potential of RNAi for pest control: a review. J. Insect Physiol. 56, 227-235. doi: 10.1016/j.jinsphys.2009.10.004
[INSECT.1] Jin, S., Singh, N. D., Li, L., Zhang, X., and Daniell, H. (2015). Engineered chloroplast dsRNA silences cytochrome p450 monooxygenase, V-ATPase and chitin synthase genes in the insect gut and disrupts Helicoverpa armigera larval developmental and pupation. Plant Biotechnol. J. 13, 435-446. doi: 10.1111/pbi.123550
[INSECT.1] Joga, M. R., Zotti, J. M., Smagghe, G., and Christiaens, O. (2016). RNAi efficiency, systemic properties, and novel delivery methods for pest insect control: what we know so far. Front. Physiol. 7:553. doi: 10.3389/fphys.2016. 00553
[INSECT.1] Katoch, R., Sethi, A., Thakur, N., and Murdock, L. L. (2013). RNAi for insect control: current perspective and future challenges. Appl. Biochem. Biotechnol. 171, 847-873. doi: 10.1007/s12010-013-0399-4
[INSECT.1] Katoch, R., and Thakur, N. (2012). Insect gut nucleases: a challenge for RNA interference mediated insect control strategies. Int. J. Biochem. Biotechnol. 1, 198-203.
[INSECT.1] Kim, Y. H., Soumaila Issa, M., Cooper, A. M., and Zhu, K. Y. (2015). RNA interference: applications and advances in insect toxicology and insect pest management. Pestic. Biochem. Physiol. 120, 109-117. doi: 10.1016/j.pestbp.2015.01.002
[INSECT.1] Li, W., Koutmou, K. S., Leahy, D. J., and Li, M. (2015). Systemic RNA Interference Deficiency-1 (SID-1) extracellular domain selectively binds long double-stranded RNA and is required for RNA transport by SID-1. J. Biol. Chem. 290, 18904-18913. doi: 10.1074/jbc.M115.658864
[INSECT.1] Mao, Y.-B., Tao, X.-Y., Xue, X.-Y., Wang, L.-J., and Chen, X.-Y. (2011). Cotton plants expressing resistance to bollworms. Transgenic Res. 20, 665-673. doi: 10.1007/s11248-010-9450-1
[INSECT.1] Merzendorfer, H. (2006). Insect chitin synthases: a review. J. Comp. Physiol. B. 176, 1-15. doi: 10.1007/s00360-005-0005-3
[INSECT.1] Merzendorfer, H., and Zimoch, L. (2003). Chitin metabolism in insects: structure, function and regulation of chitin synthases and chitinases. J. Exp. Biol. 206(Pt 24), 4393-4412. doi: 10.1242/jeb.00709
[INSECT.1] Moffat, J., Reiling, J. H., and Sabatani, D. M. (2007). Off-target effects associated with long dsRNAs in Drosophila RNAi sreens. Trends Pharmacol. Sci. 28, 149-151. doi: 10.1016/j.tips.2007.02.009
[INSECT.1] Neves, R. C. S., Colares, F., Torres, J. B., Santos, R. L., and Bastos, C. S. (2014). Rational practices to manage boll weevils colonization and population growth on family farms in the semiarido region of Brazil. Insects 5, 818-831. doi: 10.3390/insects5040818
[INSECT.1] Pan, C., Jia, W., Lu, B., and Bishop, C. E. (2014). Expression of TAT recombinant 0ct4, Sox2, Lin28, and Nanog proteins from baculovirus-infected Sf9 insect cells. Gene 556, 245-248. doi: 10.1016/j.gene.2014. 11.061[INSECT.1] Price, D. R., and Gatehouse, J. A. (2008). RNAi-mediated crop protection against insects. Trends Biotechnol. 26, 393-400. doi: 10.1016/j.tibtech.2008.04.004
[INSECT.1] Shukla, J. N., Kalsi, M., Sethi, A., Narva, K. E., Fishilevich, E., Singh, S., et al. (2016). Reduced stability and intracellular transport of dsRNA contribute to poor RNAi response in lepidopteran insects. RNA Biol. 13, 656-669. doi: 10.1080/15476286.2016.1191728
[INSECT.1] Toprak, U., Erlandson, M., Baldwin, D., Karcz, S., Wan, L., Coutu, C., et al. (2016). Identification of the Mamestra configurata (Lepidoptera: Noctuidae) peritrophic matrix proteins and enzymes involved in peritrophic matrix chitin metabolism. Insect Sci. 23, 656-674. doi: 10.1111/1744-7917.12225
[INSECT.1] Van Rij, R. P., Saleh, M. C., Berry, B., Foo, C., Houk, A., Antoniewski, C., et al. (2006). The RNA silencing endonuclease Argonaute 2 mediates specific antiviral immunity in Drosophila melanogaster. Genes Dev. 20, 2985-2995. doi: 10.1101/gad.1482006
[INSECT.1] Wynant, N., Santos, D., Verdonck, R., Spit, J., Van Wielendaele, P., and Vanden Broeck, J. (2014). Identification, functional characterization and phylogenetic analysis of double stranded RNA degrading enzymes present in the gut of the desert locust, Schistocerca gregaria. Insect Biochem. Mol. Biol. 46, 1-8. doi: 10.1016/j.ibmb.2013.12.008
[INSECT.1] Zhang, J., Khan, S. A., Hasse, C., Ruf, S., Heckel, D. G., and Bock, R. (2015). Full crop protection from an insect pest by expression of long double-stranded RNAs in plastids. Science 347, 991-994. doi: 10.1126/science.1261680
[INSECT.1] Zhang, X., Zhang, J., and Zhu, K. Y. (2010). Chitosan/double-stranded RNA nanoparticle-mediated RNA interference to silence chitin synthase genes through larval feeding in the African malaria mosquito (Anopheles gambiae). Insect Mol. Biol. 19, 683-693. doi: 10.1111/j.1365-2583.2010. 01029.x
[INSECT.1] Zhou, Z., Li, Y., Yuan, C., Zhang, Y., and Qu, L. (2015). Oral administration of TAT-PTD—diapause hormone fusion protein interferes with Helicoverpa armigera (Lepidoptera: Noctuidae) development. J. Insect Sci. 15:123. doi: 10.1093/jisesa/iev102

Rnase Citations:
[RNASE.1] Arimatsu, Y., Furuno, T., Sugimura, Y., Togoh, M., Ishihara, R., Tokizane, M., et al. (2007a). Purification and properties of double-stranded RNA degrading nuclease, dsRNase, from the digestive juice of the silkworm, Bombyx mori. J. Insect Biotechnol. Sericol. 76, 57-62. doi: 10.11416/jibs.76.1_57
[RNASE.1] Arimatsu, Y., Kotani, E., Sugimura, Y., and Furusawa, T. (2007b). Molecular characterization of a cDNA encoding extracellular dsRNase and its expression in the silkworm, Bombyx mori. Insect Biochem. Mol. Biol. 37, 176-183. doi: 10.1016/j.ibmb.2006.11.004

Oral Delivery Citations:
[ORAL.1] Kwon, K.-C., and Daniell, H. (2016). Oral delivery of protein drugs bioencapsulated in plant cells. Mol. Ther. 24, 1342-1350. doi: 10.1038/mt.2016.115
[ORAL.1] Park, E. Y., Jang, M., Kim, J. H., and Ahn, H. J. (2014). Genetically modified tomato aspermy virus 2b protein as a tumor-targeting siRNA delivery carrier. Acta Biomater. 10, 4778-4786. doi: 10.1016/j.actbio.2014.07.014

RNAi Off-Target Citations:
[OFF.1] Kulkarni, M., Booker, M., Serena, J. S., Friedman, A., Pengyu, H., Norbert, P., et al. (2006). Evidence of off-target effects associated with long dsRNAs in Drosophila melanogaster cell-based assays. Nat. Methods 3, 833-838. doi: 10.1038/nmeth935
[OFF.1] Qiu, S., Adema, C. M., and Terran, L. (2005). A computational study of off-target effects of RNA interference. Nucleic Acids Res. 33, 1834-1847. doi: 10.1093/nar/gki324

Double-Stranded Binding Domain Citations:
[DSRBD.1] Geoghegan, J. C., Gilmore, B. L., and Davidson, B. L. (2012). Gene silencing mediated by siRNA-binding fusion proteins is attenuated by double-stranded RNA-binding domain structure. Mol. Ther. Nucleic Acids. 1:e53. doi: 10.1038/mtna.2012.43
[DSRBD.1] Kimberlin, C. R., Bornholdt, Z. A., Li, S., Woods, V. L., MacRae, I. J., and Saphire, E. O. (2010). Ebolavirus VP35 uses a bimodal strategy to bind dsRNA for innate immune suppression. Proc. Natl. Acad. Sci. U.S.A. 107, 314-319. doi: 10.1073/pnas.0910547107
[DSRBD.1] Koukiekolo, R., Sagan S. M., and Pezacki J. P. (2007). Effects of pH and salt concentration on the siRNA binding activity of the RNA silencing suppressor protein p19. FEBS Lett. 581, 3051-3056. doi: 10.1016/j.febslet.2007.05.064

Misc Citations:
[GEL.1] Hellman, L. M., and Fried, M. G. (2007). Electrophoretic mobility shift assay (EMSA) for detecting protein-nucleic acid interactions. Nat. Protoc. 2, 1849-1861. doi: 10.1038/nprot.2007.249

What is claimed is:

1. (canceled)

2. The polynucleotide-moiety complex of claim 9, wherein the one or more surface moieties is a peptide, small molecule, metabolite, organic chemical, inorganic chemical, precursor protein, or a protein other than a viral coat protein.

3. The polynucleotide-moiety complex of claim 9, wherein the one or more aptamers or intramers is at least four aptamers or intramers.

4. The polynucleotide-moiety complex of claim 9, wherein the core has a diameter of about 30-60 nm, about 60-200 nm, or about 60-300 nm.

5. The polynucleotide-moiety complex of claim 9, wherein the core has a diameter of about 20 nm, about 40 nm, or about 40-200 nm.

6. The polynucleotide-moiety complex of claim 9, wherein the core comprises natural or synthetic RNA or DNA.

7. The polynucleotide-moiety complex of claim 9, wherein the polynucleotide-moiety complex is expressed and moiety-coated within a host cell selected from a plant cell, yeast cell, bacterial cell, human cell, or animal cell, or by in-vitro transcription.

8. The polynucleotide-moiety complex of claim 9, wherein the polynucleotide-moiety complex is expressed and moiety-coated within a host cell and the core targets genes other than the host.

9. A polynucleotide-moiety complex comprising:
(a) a single-stranded self-forming polynucleotide nanoparticle core comprising:
(i) a plurality of multivalent RNA (MV-RNA) molecules connected to each other by one or more nucleotides and
(ii) one or more aptamers or intramers, capable of binding one or more surface moieties; and
(b) a shell comprising one or more surface moieties bound to the one or more aptamers or intramers.

10. The polynucleotide-moiety complex of claim 9, wherein the surface charge of the complex at a physiological pH is different than of the core if isolated.

11. The polynucleotide-moiety complex of claim 9, wherein the molecular weight of the complex at a physiological pH is different than of the core if isolated.

12. The polynucleotide-moiety complex of claim 9, wherein the size of the complex at a physiological pH is different than of the core if isolated.

13. The polynucleotide-moiety complex of claim 9, wherein the hydrophobicity of the complex at a physiological pH is different than of the core if isolated.

14. The polynucleotide-moiety complex of claim 9, wherein the mode of action of the complex is different than of the core if isolated.

15. The polynucleotide-moiety complex of claim 9, wherein the nuclease degradation resistance of the complex is different than of the core if isolated.

16. The polynucleotide-moiety complex of claim 9, wherein the one or more surface moieties mode of action is antagonized.

17. The polynucleotide-moiety complex of claim 9, wherein the one or more surface moieties mode of action is agonized.

18. The polynucleotide-moiety complex of claim 9, wherein the bioavailability of the complex is different than of the core if isolated.

19. The polynucleotide-moiety complex of claim 9, wherein the mode of cellular uptake of the complex is different than of the core if isolated.
